SEQUENCE LISTING (26 sequences)

SEQ ID NO: 1 - protein, 593 aa, Streptococcus sp.
MEKEKKVKYFLRKSAFGLASVSAAFLVGSTVFAVDSPIEDTPIIRNGGELTNLLGNSETTLALRNEESATADLTAAAVADTVAAAAAENAGAAAWEAAAAADALAKAKADALKEFNKYGVSDYYKNLINNAKTVEGVKDLQAQVVESAKKARISEATDGLSDFLKSQTPAEDTVKSIELAEAKVLANRELDKYGVSDYHKNLINNAKTVEGVKDLQAQVVESAKKARISEATDGLSDFLKSQTPAEDTVKSIELAEAKVLANRELDKYGVSDYYKNLINNAKTVEGVKALIDEILAALPKTDTYKLILNGKTLKGETTTEAVDAATAEKVFKQYANDNGVDGEWTYDDATKTFTVTEKPEVIDASELTPAVTTYKLVINGKTLKGETTTEAVDAATAEKVFKQYANDNGVDGEWTYDDATKTFTVTEKPEVIDASELTPAVTTYKLVINGKTLKGETTTKAVDAETAEKAFKQYANDNGVDGVWTYDDATKTFTVTEMVTEVPGDAPTEPEKPEASIPLVPLTPATPIAKDDAKKDDTKKEDAKKPEAKKEDAKKAETLPTTGEGSNPFFTAAALAVMAGAGALAVASKRKED

SEQ ID NO: 2 - protein, 454 aa, Staphylococcus aureus
MMTLQIHTGGINLKKKNIYSIRKLGVGIASVTLGTLLISGGVTPAANAAQHDEAQQNAFYQVLNMPNLNADQRNGFIQSLKDDPSQSANVLGEAQKLNDSQAPKADAQQNKFNKDQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPGKEDNNKPGKEDGNKPGKEDNKKPGKEDGNKPGKEDNKKPGKEDGNKPGKEDGNKPGKEDGNKPGKEDGNGVHVVKPGDTVNDIAKANGTTADKIAVDNKLADKNMIKPGQELVVDKKQPANHADANKAQALPETGEENPFIGTTVFGGLSLALGAALLAGRRREL

SEQ ID NO: 3 - protein, 719 aa, Peptostreptococcus magnus
MAALAGAIVVTGGVGSYAADEPIDLEKLEEKRDKENVGNLPKFDNEVKDGSENPMAKYPDFDDEASTRFETENNEFEEKKVVSDNFFDQSEHPFVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAGKEEPEETPEKPEVQDGYASYEEAEAAAKEALKNDDVNKSYTIRQGADGRYYYVLSPVEAEEEKPEAQNGYATYEEAEAAAKKALENDPINKSYSIRQGADGRYYYVLSPVEAETPEKPVEPSEPSTPDVPSNPSNPSTPDVPSTPDVPSNPSTPEVPSNPSTPGNEEKPGNEQKPGNEQKPGNEQKPGNEQKPGNEQKPDQPSKPEKEENGKGGVDSPKKKEKAALPKAGSEAEILTLAAASLSSVAGAFISLKKRK

SEQ ID NO: 4 - protein, 20 aa, artificial sequence
  protein A mimetic polypeptide
EQQNAFYEILHLPNLNEEQR

SEQ ID NO: 5 - protein, 16 aa, Artificial
  protein A mimetic polypeptide
RTYRTYRTYRTYKKKG

SEQ ID NO: 6 - protein, 18 aa, Mycobacterium tuberculosis
FEAVKGECNMGQQEIGFR

SEQ ID NO: 7 - protein, 15 aa, Mycobacterium tuberculosis
EIADQHGKSLTFMAK

SEQ ID NO: 8 - protein, 14 aa, Mycobacterium tuberculosis
EFTLCYAPTINSYK

SEQ ID NO: 9 - protein, 9 aa, Mycobacterium tuberculosis
VVGHGQNIR

SEQ ID NO: 10 - protein, 19 aa, Mycobacterium tuberculosis
LPVTLADAAVLFEDSALVR

SEQ ID NO: 11 - protein, 15 aa, Mycobacterium tuberculosis
VELAAFNAAVTDWER

SEQ ID NO: 12 - protein, 457 aa, Mycobacterium tuberculosis
MTGPGSPPLAWTELERLVAAGDVDTVIVAFTDMQGRLAGKRISGRHFVDDIATRGVECCSYLLAVDVDLNTVPGYAMASWDTGYGDMVMTPDLSTLRLIPWLPGTALVIADLVWADGSEVAVSPRSILRRQLDRLKARGLVADVATELEFIVFDQPYRQAWASGYRGLTPASDYNIDYAILASSRMEPLLRDIRLGMAGAGLRFEAVKGECNMGQQEIGFRYDEALVTCDNHAIYKNGAKEIADQHGKSLTFMAKYDEREGNSCHIHVSLRGTDGSAVFADSNGPHGMSSMFRSFVAGQLATLREFTLCYAPTINSYKRFADSSFAPTALAWGLDNRTCALRVVGHGQNIRVECRVPGGDVNQYLAVAALIAGGLYGIERGLQLPEPCVGNAYQGADVERLPVTLADAAVLFEDSALVREAFGEDVVAHYLNNARVELAAFNAAVTDWERIRGFERL

SEQ ID NO: 13 - DNA, 1191 bp, Mycobacterium tuberculosis
  CDS (1)..(1191)
gtggcgaaggcgaagttccagcggaccaagccccacgtcaacatcgggaccatcggtcacgttgaccacggcaagaccaccctgaccgcggctatcaccaaggtcctgcacgacaaattccccgatctgaacgagacgaaggcattcgaccagatcgacaacgcccccgaggagcgtcagcgcggtatcaccatcaacatcgcgcacgtggagtaccagaccgacaagcggcactacgcacacgtcgacgcccctggccacgccgactacatcaagaacatgatcaccggcgccgcgcagatggacggtgcgatcctggtggtcgccgccaccgacggcccgatgccccagacccgcgagcacgttctgctggcgcgtcaagtgggtgtgccctacatcctggtagcgctgaacaaggccgacgcagtggacgacgaggagctgctcgaactcgtcgagatggaggtccgcgagctgctggctgcccaggaattcgacgaggacgccccggttgtgcgggtctcggcgctcaaggcgctcgagggtgacgcgaagtgggttgcctctgtcgaggaactgatgaacgcggtcgacgagtcgattccggacccggtccgcgagaccgacaagccgttcctgatgccggtcgaggacgtcttcaccattaccggccgcggaaccgtggtcaccggacgtgtggagcgcggcgtgatcaacgtgaacgaggaagttgagatcgtcggcattcgcccatcgaccaccaagaccaccgtcaccggtgtggagatgttccgcaagctgctcgaccagggccaggcgggcgacaacgttggtttgctgctgcggggcgtcaagcgcgaggacgtcgagcgtggccaggttgtcaccaagcccggcaccaccacgccgcacaccgagttcgaaggccaggtctacatcctgtccaaggacgagggcggccggcacacgccgttcttcaacaactaccgtccgcagttctacttccgcaccaccgacgtgaccggtgtggtgacactgccggagggcaccgagatggtgatgcccggtgacaacaccaacatctcggtgaagttgatccagcccgtcgccatggacgaaggtctgcgtttcgcgatccgcgagggtggccgcaccgtgggcgccggccgggtcaccaagatcatcaagtag

SEQ ID NO: 14 - protein, 396 aa, Mycobacterium tuberculosis
VAKAKFQRTKPHVNIGTIGHVDHGKTTLTAAITKVLHDKFPDLNETKAFDQIDNAPEERQRGITINIAHVEYQTDKRHYAHVDAPGHADYIKNMITGAAQMDGAILVVAATDGPMPQTREHVLLARQVGVPYILVALNKADAVDDEELLELVEMEVRELLAAQEFDEDAPVVRVSALKALEGDAKWVASVEELMNAVDESIPDPVRETDKPFLMPVEDVFTITGRGTVVTGRVERGVINVNEEVEIVGIRPSTTKTTVTGVEMFRKLLDQGQAGDNVGLLLRGVKREDVERGQVVTKPGTTTPHTEFEGQVYILSKDEGGRHTPFFNNYRPQFYFRTTDVTGVVTLPEGTEMVMPGDNTNISVKLIQPVAMDEGLRFAIREGGRTVGAGRVTKIIK

SEQ ID NO: 15 - protein, 17 aa, Mycobacterium tuberculosis
KLLDQGQAGDNVGLLLR

SEQ ID NO: 16 - protein, 7 aa, Pseudomonas aeruginosa
LRPLHDR

SEQ ID NO: 17 - protein, 10 aa, Pseudomonas aeruginosa
GEVVAVGTGR

SEQ ID NO: 18 - protein, 9 aa, Pseudomonas aeruginosa
MKLRPLHDR

SEQ ID NO: 19 - protein, 9 aa, Pseudomonas aeruginosa
MKLRPLHDR

SEQ ID NO: 20 - protein, 13 aa, Pseudomonas aeruginosa
VVFGPYSGSNAIK

SEQ ID NO: 21 - protein, 11 aa, Pseudomonas aeruginosa
LRPLHDRVVIR

SEQ ID NO: 22 - protein, 13 aa, Pseudomonas aeruginosa
VLDNGEVRALAVK

SEQ ID NO: 23 - protein, 17 aa, Pseudomonas aeruginosa
TAGGIVLPGSAAEKPNR

SEQ ID NO: 24 - protein, 17 aa, Pseudomonas aeruginosa
VGDKVVFGPYSGSNAIK

SEQ ID NO: 25 - protein, 97 aa, Pseudomonas aeruginosa
MKLRPLHDRVVIRRSEEETKTAGGIVLPGSAAEKPNRGEVVAVGTGRVLDNGEVRALAVKVGDKVVFGPYSGSNAIKVDGEELLVMGESEILAVLED

SEQ ID NO: 26 - protein, 114 aa, homo sapiens
MTCKMSQLERNIETIINTFHQYSVKLGHPDTLNQGEFKELVRKDLQNFLKKENKNEKVIEHIMEDLDTNADKQLSFEEFIMLMARLTWASHEKMHEGDEGPGHHHKPGLGEGTP

(19) United States
(12) Patent Application Publication
(10) Pub. No.: US 2007/0178541 A1
(43) Pub. Date: Aug. 2, 2007

(54) Method of isolating a protein

(75) Inventors: Susanne Kartin Pedersen, Lilyfield (AU); Robert Alan Cole, Greenwich (AU); Ron Peter Weinberger, North Boudi (AU); Andrew John Sloane, Balmain (AU)

Correspondence Address: COZEN O'CONNOR, P.C., 1900 MARKET STREET, PHILADELPHIA, PA, 19103-3508, US

(73) Assignee: PROTEOME SYSTEMS INTELLECTUAL PROPERTY PTY LTD, North Ryde (AU)

(21) Appl. No.: 10/562,132

(22) PCT Filed: Jun. 28, 2004
(86) PCT No.: PCT/AU04/00856

§ 371 Date: Dec. 6, 2006

(30) Foreign Application Priority Data
Jun. 27, 2003 (AU) 2003903317

(51) Int. Cl. C12Q 1/70; G01N 33/569; G01N 33/554; G01N 33/543

(52) U.S. Cl. 435/7.32; 436/518; 435/5; 435/7.31

(57) ABSTRACT

The present invention provides a method for isolating and/or identifying an immunogenic protein from a protein complex comprising an immunoglobulin or a mixture thereof or an immunoglobulin-containing fraction.

### FIELD OF THE INVENTION

The present invention relates to a novel method of identifying and isolating one or more proteins from a biological sample of a human or animal subject, wherein the biological sample comprises a protein complex comprising an immunoglobulin and a protein to be isolated or identified bound to said immunoglobulin or a mixture of immunoglobulins by virtue of one or more antibody-antigen interactions, or an immunoglobulin-containing fraction comprising the protein to be isolated bound to one or more immunoglobulins. The present invention clearly encompasses the separation of the protein of interest from the immunoglobulin fraction. The present invention also encompasses the partial or complete enrichment or purification of a protein of interest by immunocapture of the immunoglobulin fraction and eluting or otherwise removing unbound protein, and optionally isolating or recovering the bound protein of interest from the captured immunoglobulin.

### BACKGROUND OF THE INVENTION

### General Information

This specification contains nucleotide and amino acid sequence information prepared using PatentIn Version 3.1. Each nucleotide sequence is identified in the sequence listing by the numeric indicator <210> followed by the sequence identifier (e.g. <210>1, <210>2, <210>3, etc). The length and type of sequence (DNA, protein (PRT), etc), and source organism for each nucleotide sequence, are indicated by information provided in the numeric indicator fields <211>, <212> and <213>, respectively. Nucleotide sequences referred to in the specification are defined by the term “SEQ ID NO:”, followed by the sequence identifier (eg. SEQ ID NO: 1 refers to the sequence in the sequence listing designated as <400>1).

The designation of nucleotide residues referred to herein are those recommended by the IUPAC-IUB Biochemical Nomenclature Commission, wherein A represents Adenine, C represents Cytosine, G represents Guanine, T represents thymine, Y represents a pyrimidine residue, R represents a purine residue, M represents Adenine or Cytosine, K represents Guanine or Thymine, S represents Guanine or Cytosine, W represents Adenine or Thymine, H represents a nucleotide other than Guanine, B represents a nucleotide other than Adenine, V represents a nucleotide other than Thymine, D represents a nucleotide other than Cytosine and N represents any nucleotide residue.

As used herein the term “derived from” shall be taken to indicate that a specified integer may be obtained from a particular source albeit not necessarily directly from that source.

Unless the context requires otherwise or specifically stated to the contrary, integers, steps, or elements of the invention recited herein as singular integers, steps or elements clearly encompass both singular and plural forms of the recited integers, steps or elements.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Unless specifically stated otherwise, each feature described herein with regard to a specific aspect or embodiment of the invention, shall be taken to apply mutatis mutandis to each and every other aspect or embodiment of the invention.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations or any two or more of said steps or features.

The present invention is not to be limited in scope by the specific embodiments described herein. Functionally-equivalent products, compositions and methods are clearly within the scope of the invention, as described herein.

All the references cited in this application are specifically incorporated by reference herein.

The present invention is performed without undue experimentation using, unless otherwise indicated, conventional techniques of molecular biology, microbiology, virology, recombinant DNA technology, peptide synthesis in solution, solid phase peptide synthesis, and immunology. Such procedures are described, for example, in the following texts that are incorporated by reference:
Sambrook, Fritsch & Maniatis, Molecular Cloning: A Laboratory Manual, Cold Spring Harbor Laboratories, New York, Second Edition (1989), whole of Vols I, II, and III;
DNA Cloning: A Practical Approach, Vols. I and II (D. N. Glover, ed., 1985), IRL Press, Oxford, whole of text;
Oligonucleotide Synthesis: A Practical Approach (M. J. Gait, ed., 1984) IRL Press, Oxford, whole of text, and particularly the papers therein by Gait, pp 1-22; Atkinson et al., pp 35-81; Sproat et al., pp 83-115; and Wu et al., pp 135-151;
Nucleic Acid Hybridization: A Practical Approach (B. D. Hames & S. J. Higgins, eds., 1985) IRL Press, Oxford, whole of text;
Immobilized Cells and Enzymes: A Practical Approach (1986) IRL Press, Oxford, whole of text;
Perbal, B., A Practical Guide to Molecular Cloning (1984);
Methods In Enzymology (S. Colowick and N. Kaplan, eds., Academic Press, Inc.), whole of series;
J. F. Ramalho Ortigão, “The Chemistry of Peptide Synthesis” In: Knowledge database of Access to Virtual Laboratory website (Interactiva, Germany);
Sakakibara, D., Teichman, J., Lien, E. Land Fenichel, R. L. (1976). Biochem. Biophys. Res. Commun. 73 336-342
Merrifield, R. B. (1963). J. Am. Chem. Soc. 85, 2149-2154.
Barany, G. and Merrifield, R. B. (1979) in The Peptides (Gross, E. and Meienhofer, J. eds.), vol. 2, pp. 1-284, Academic Press, New York.
Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart.
Bodanszky, M. (1984) Principles of Peptide Synthesis, Springer-Verlag, Heidelberg.
Bodanszky, M. & Bodanszky, A. (1984) The Practice of Peptide Synthesis, Springer-Verlag, Heidelberg.
Bodanszky, M. (1985) Int. J. Peptide Protein Res. 25, 449-474.
Handbook of Experimental Immunology, Vols. I-IV (D. M. Weir and C. C. Blackwell, eds., 1986, Blackwell Scientific Publications).

### DESCRIPTION OF THE RELATED ART

As a response to the increasing demand for new diagnostic targets, lead compounds and new target identification and validation reagents, the pharmaceutical industry has increased its screening for new markers or compounds specific to pathogenic organisms or disease states, such as, for example, in the diagnosis/prognosis and/or treatment of infection or autoimmune disease.

As many pathogenic organisms express unique proteins or isoforms of proteins, much research has focussed on identifying and using these proteins in the development of novel diagnostic/prognostic and/or therapeutic strategies. However, not all proteins expressed by a pathogenic organism represent suitable targets for use in a method of diagnosis or in a therapeutic strategy. Accordingly, a large amount of the research in identifying new diagnostic and/or therapeutic strategies is directed toward the identification of suitable target molecules.

Perhaps the simplest approach in identifying a diagnostic/prognostic target of interest is to determine a protein derived from a pathogen wherein the pathogen is associated with a disease or condition, or alternatively, to determine a host cell protein having an altered expression pattern as a consequence of the disease or conditions. The determined protein is then used to generate or identify an antibody that is able to specifically bind to said protein or a region thereof, to determine whether or not the protein is sufficiently immunogenic to facilitate its use in the preparation of immuno-diagnostic reagents. However, such methods experience a high rate of failure as many of the proteins that are tested are not immunogenic or at least not to the degree required to elicit an immune response in a host, for the production of immuno-diagnostic reagents and kits or vaccines.

Furthermore, a target of an antibody, ligand or small molecule may be relatively inaccessible in the native environment, ie in a complex with other proteins or within a cell, thereby hindering its detection by immunoassay.

Accordingly, the high failure rate of such a method means that this approach is both laborious and expensive, as often several potential targets must be tested before a putative target is identified.

With the completion of the sequencing of the genome of several pathogenic organisms researchers have commenced using this information to attempt to predict the function of proteins that are expressed by these pathogenic organisms. Using both functional and sequence information researchers attempt to predict the location and accessibility of proteins expressed by the pathogenic organism, and thus the likelihood that a protein represents a diagnostic or therapeutic target for the treatment of an infectious organism. As reported by Masignani et al, Expert Opin. Biol. Ther. 2(8), 895-905, 2002, this process can lead to the rapid prediction of putative diagnostic, therapeutic and/or vaccine targets, leading to an acceleration of the development of new therapeutic/diagnostic opportunities.

Methods that depend upon analysis of the genome sequence of an organism require that the genome, or at least a significant proportion of the genome of the organism of interest has been sequenced. Accordingly, this method is ineffective at predicting diagnostic/therapeutic targets in organisms that have genomes that are yet to be sequenced, especially in those organisms that have only been recently identified. Furthermore, such an approach is of limited use in the prediction of potential therapeutic/diagnostic targets in pathogens, such as, for example, some retroviruses, which maintain a high mutation rate, thus regularly changing their genomic sequence.

Furthermore, these methods require the skilled artisan to determine which, if any, of the predicted target proteins are actually expressed by the pathogenic organism in vivo. This is made more difficult with the observation that some proteins are only expressed at certain stages of a disease or disorder. Accordingly, those proteins expressed, for example, late in an infection may be of limited use in the context of an early diagnostic or a vaccine.

As can be perceived from the preceding discussion, there remains a need for a method of for the rapid isolation and/or identification of a protein that is an attractive target for a diagnostic assay and/or method of treatment.

### SUMMARY OF THE INVENTION

In work leading up to the present invention the inventors sought to identify novel diagnostic, vaccine and drug target proteins for infection by Mycobacterium tuberculosis or Pseudomonas aeruginosa. The inventors found that they were able to recover immunogenic target proteins or peptide fragments from the immunoglobulin (Ig) fraction of patient serum samples, where proteins or peptide fragments were sufficiently non-degraded to permit determination of their amino acid sequences. This was surprising as proteins are known to be rapidly degraded during infection. Accordingly, the inventors proceeded against conventional wisdom in the art, by identifying several proteins/fragments from the Ig fraction of serum obtained from patients suffering from tuberculosis.

Using this method, the present inventors identified the M. tuberculosis glutamine synthetase (Rv2860c) from the IgG fraction of sputum and sera from TB patients. They also used the described immunoseparation techniques to isolate and identify the M. tuberculosis protein Elongation factor Tu (Rv 0685) from the IgG fraction of sera from a TB patient.

The present inventors have further developed the method of the present invention by capturing an immunoglobulin-containing fraction from a subject suffering from an infection. The captured immunoglobulin-containing fraction is then immobilised and contacted with, for example, a body fluid from a subject infected with the infectious organism, or a cell or a cell extract. In this way the subject's Ig fraction is used to purify an immunogenic protein from an infectious organism. By eluting or isolating bound proteins from the immobilized Ig fraction the present inventors have been able to capture increased levels of immunogenic proteins, thereby facilitating identification and analysis of said proteins. Using an Ig fraction from a subject suffering from CF that is suffering from P. aeruginosa infection the present inventors have isolated several immunogenic proteins from said bacteria, including the heat shock protein GroES.

Furthermore, the present inventors have adapted the previously described method to isolate proteins from sputum of a CF subject against which a CF subject suffering from an acute clinical exacerbation had developed auto-antibodies. Accordingly, the method is useful for identifying a protein against which a subject suffering from an autoimmune condition has raised a specific immune response.

The present inventors have further developed the subject method by immunizing a subject with protein complement from an infectious organism. An immunoglobulin-containing fraction is isolated from said animal or from a sample produced by the subject is then isolated and immobilized. This immobilized Ig fraction is then contacted with the body fluids of a patient infected with like organism and the and the in vivo expressed proteins captured, eluted and identified, thereby determining an in vivo expressed protein from the infectious organism against which a subject raises an antibody response.

The approaches taken by the inventors are of general use in the identification of any immunogenic protein. Such an immunogenic protein represents an attractive target and is useful, for example, for diagnostic applications to identify a pathogenic organism or infectious state or an autoimmune state in a subject. Additionally, such a protein is also useful for developing therapeutic or prophylactic strategies for the treatment of an infection by a pathogenic organism from which said protein is isolated or an autoimmune state in a subject.

This invention provides, a method for identifying an immunogenic protein or fragment thereof capable of eliciting an immune response, said method comprising obtaining a protein complex comprising an immunoglobulin or mixtures thereof or an immunoglobulin-containing fraction from a subject or a cell, tissue or organ thereof and identifying a protein or fragment thereof bound to the immunoglobulin by virtue of an antigen-antibody interaction, thereby identifying an immunogenic protein or fragment thereof capable of eliciting an immune response.

The invention further provides for, eg., obtaining a sample from the subject that comprises the protein complex or mixture thereof or immunoglobulin-containing fraction. The invention further provides for obtaining one or more immunoglobulin-containing fractions from the sample.

For example, the sample is selected from the group consisting of whole blood, plasma, serum, sputum, saliva, pleural fluid, pericardial fluid, peritoneal fluid, lymph fluid, lymph node, spleen, egg yolk, a fraction of whole blood, a fraction of plasma, a fraction of serum, a fraction of sputum, a fraction of saliva, a fraction of pleural fluid, a fraction of pericardial fluid, a fraction of peritoneal fluid, a fraction of lymph fluid, a fraction of lymph node, a fraction of spleen and a fraction of egg yolk. In another example, the sample comprises a cell selected from the group consisting of peripheral blood mononuclear cell (PBMC), lymphocyte, B-lymphocyte, T lymphocyte, helper T-cell, cytotoxic T cell, macrophage, dendritic cell, polymorphonuclear cell and mast cell. In yet another example, the sample comprises serum or an immunoglobulin-containing fraction of serum.

In one example, the protein complex or immunoglobulin-containing fraction thereof comprises one or more immunoglobulins selected from the group consisting of IgM, IgG, IgA, IgE, IgD and IgY or mixtures thereof. For example, the protein complex or immunoglobulin-containing thereof comprises IgG. Alternatively, or in addition, the protein complex or an immunoglobulin-containing fraction comprises IgA.

Such a protein complex or immunoglobulin-containing fraction is obtained, for example, by a process comprising separating or purifying a sample from the subject to thereby provide said protein complex or immunoglobulin-containing fraction. For example, said separating or purifying a sample from the subject comprises contacting the sample with one or more compounds capable of binding an immunoglobulin for a time and under conditions sufficient for binding to occur and isolating the compound.

For example, the compound is previously immobilized on a solid support, matrix or resin, eg., the solid support, matrix or resin is selected from the group consisting of cellulose bead, agarose, nylon, magnetic particle, paramagnetic particle, polymeric resin and mixtures thereof.

The method of the invention additionally provides for washing the one or more immobilized compounds to thereby remove non-specifically bound or unbound protein.

In one example, a compound is selected from the group consisting of Protein A or a mimetic thereof, Protein G or a mimetic thereof, Protein L or a mimetic thereof, an anti-immunoglobulin antibody, a maltose binding protein (MBP) and a thiophilic resin or mixtures thereof. For instance, the compound is Protein A, Protein G or mixtures thereof.

In an example of the method of the present invention, the subject suffers from an infection or has suffered previously from an infection. For example, the infection is an acute infection or a chronic infection. Such an infection is, for example, selected from the group consisting of a viral infection, a bacterial infection, a yeast infection, a fungal infection, a parasitic infection. For instance, the infection is a bacterial infection, eg. a Pseudomonas infection or a Mycobacterium infection.

In another example, the infection is a pulmonary infection, eg., a pulmonary infection caused by or associated with the presence of Pseudomonas aeruginosa or Mycobacterium tuberculosis (eg. tuberculosis).

In a further example, the subject suffers from an autoimmune condition, eg., an autoimmune condition that is associated with an inflammatory condition.

In a still further example, the method additionally comprises immunizing a subject with one or more cells or an extract thereof comprising the immunogenic protein or fragment thereof to thereby elicit an immune response to the immunogenic protein or fragment thereof (e.g. in the subject or a sample derived from or produced by the subject). For instance, the one or more cells or extract thereof is derived from an infectious agent expressing the immunogenic protein or fragment thereof. Examples of a useful cell or cell extract are selected from the group consisting of a viral particle, a bacterial cell, a yeast cell, a fungal cell or a cell of or derived from a parasite or the cellular extract is selected from the group consisting of an extract from a virus, an extract from a bacterium, an extract from a yeast, an extract from a fungus and an extract from a parasite. For instance, the one or more cells are bacterial cells or the cellular extract is a bacterial extract, eg., a Pseudomonas sp., eg., Pseudomonas aeruginosa or Mycobacterium e.g., M. tuberculosis.

In an example of the present invention, the subject is a non-human animal, eg., the non-human animal is selected from the group consisting of mouse, rat, rabbit, chicken, dog, sheep, ovine, horse and goat.

In another example, the subject is a human subject.

The method of the present invention additionally provides for linking immunoglobulin to the one or more compounds, eg., by performing a process that comprises contacting cross-linking agent with the one or more compounds having immunoglobulin bound hereto for a time and under conditions sufficient for covalent linkage to occur between compound and immunoglobulin.

Such a cross-linking agent is, for example, selected from the group consisting of an imidoester cross-linker, a N-hydroxysuccinimide cross-linker, a maleimide cross-linker, a haloacetyl cross-linker, a hydrazide cross-linker, and a carbodiimide cross-linker. For instance, the cross linking agent is a N-hydroxysuccinimide cross-linker, eg., the N-hydroxysuccinimide cross-linker is selected from the group consisting of disuccinimidyl glutarate, disuccinimidyl suberate, bis(sulfosuccinimidyl)suberate, dithiobis(succinimidyl propionate), 3,3′-dithiobis(succinimidyl propionate), ethylene glycobis(succinimidyl succinate), ethylene glycobis(sulfo-succinimidylsuccinate), disuccinimidyl tartarate, disulfosuccinimidyl tartarate, bis[2-(succinimidyloxy-carbonyloxy) ethyl]sulfone, bis[2-(sulfosuccinimidyloxy-carbonyloxy) ethyl]sulfone, succinimidyl 4-(N-maleimidomethyl) cyclohexane-1-carboxylate, sulfo-succinimidyl 4-(N-maleimidomethyl) cyclohexane-1-carboxylate, m-maleimido benzoyl-N-hydroxysuccinimide ester, m-maleimido benzoyl-N-hydroxysulfo succinimide ester, succinimidyl 4-(p-maleimidophenyl)-butyrate, sulfo-succinimidyl 4-(p-maleimidophenyl)-butyrate, bismaleimidohexane, N-(g-maleimidobutyryloxy)succinimide ester and N-(g-maleimidobutyryloxy)sulfosuccinimide ester. For example, the N-hydroxysuccinimide cross-linker is disuccinimidyl suberate.

The invention also provides a method for identifying an immunogenic protein or an immunogenic protein fragment of an agent that causes a disease or disorder in a subject comprising:
(i) obtaining a protein complex comprising an immunoglobulin or mixtures thereof or an immunoglobulin-containing fraction from a subject suffering from the disease or disorder or having suffered previously from the disease or disorder or a cell, tissue or organ thereof;
(ii) contacting immunoglobulin in the protein complex or immunoglobulin-containing fraction with a sample comprising the agent that causes the disease or disorder or a derivative thereof; and
(ii) identifying a protein or fragment thereof bound to said immunoglobulin by virtue of an antigen-antibody interaction,
wherein the identified protein is an immunogenic protein or immunogenic protein fragment of an agent that causes a disease or disorder in a subject.

In one instance, the derivative comprises a protein or cellular extract of the agent that causes the disease or disorder, for example, an infectious agent, eg., an infectious agent selected from the group consisting of a virus infection, a bacterium, a yeast, a fungus and a parasite. For example, the infectious agent is a bacterium, eg., Pseudomonas aeruginosa or Mycobacterium tuberculosis. For instance, the bacterium is a clinical isolate.

The present invention a additionally provides a method for identifying an immunogenic protein or immunogenic protein fragment of a cancer cell comprising:
(i) obtaining a protein complex comprising an immunoglobulin or mixtures thereof or an immunoglobulin-containing fraction from a subject suffering from cancer or having suffered previously from cancer;
(ii) contacting immunoglobulin in the protein complex or immunoglobulin-containing fraction with a sample comprising the tumor cell or a protein extract or cellular extract thereof; and
(ii) identifying a protein or fragment thereof bound to said immunoglobulin by virtue of an antigen-antibody interaction,
wherein the identified protein is an immunogenic protein or immunogenic protein fragment of the cancer cell.

For example, the cancer cell selected from the group consisting of a bladder cancer cell, a breast cancer cell, a colorectal cancer cell, an endometrial cancer cell, a head and neck cancer cell, a leukemia cell, a lung cancer cell, a lymphoma cell, a melanoma cell, a non-small-cell lung cancer cell, an ovarian cancer cell, a prostate cancer cell, an acute lymphocytic leukemia cell, an adult acute myeloid leukemia cell, an adult non-Hodgkin's lymphoma cell, a brain tumor cell, a cervical cancer cell, a childhood sarcoma cell, a chronic lymphocytic leukemia cell, a chronic myeloid leukemia cell, an oesophageal cancer cell, a hairy cell leukemia cell, a kidney cancer cell, a liver cancer cell, a multiple myeloma cell, a neuroblastoma cell, an oral cancer cell, a pancreatic cancer cell, a primary central nervous system lymphoma cell, a skin cancer cell and a small-cell lung cancer cell

The invention further provides for, eg., obtaining a sample from the subject that comprises the protein complex or mixture thereof or immunoglobulin-containing fraction thereof. The invention even further provides for obtaining one or more immunoglobulin-containing fractions from the sample.

For example, the sample is selected from the group consisting of whole blood, plasma, serum, sputum, saliva, pleural fluid, pericardial fluid, peritoneal fluid, lymph fluid, lymph node, spleen, egg yolk a fraction of whole blood, a fraction of plasma, a fraction of serum, a fraction of sputum, a fraction of saliva, a fraction of pleural fluid, a fraction of pericardial fluid, a fraction of peritoneal fluid, a fraction of lymph fluid, a fraction of lymph node, a fraction of spleen and a fraction of egg yolk. In another example, the sample comprises a cell selected from the group consisting of peripheral blood mononuclear cell (PBMC), lymphocyte, B-lymphocyte, T lymphocyte, helper T-cell, cytotoxic T cell, macrophage, dendritic cell, polymorphonuclear cell and mast cell. In yet another example, the sample comprises serum or an immunoglobulin-containing fraction of serum.

In one example, the protein complex or immunoglobulin-containing fraction comprises one or more immunoglobulins selected from the group consisting of IgM, IgG, IgA, IgE, IgD and IgY or mixtures thereof. For example, the protein complexing or immunoglobulin-containing fraction comprises IgG. Alternatively, or in addition, the protein complexing or immunoglobulin-containing fraction comprises IgA. Alternatively, or in addition, the protein complexing or immunoglobulin-containing fraction comprising IgM.

The invention also provides for obtaining the protein complexing or immunoglobulin-containing fraction by a process comprising separating or purifying a sample from the subject to thereby provide said protein complexing or immunoglobulin-containing fraction. For example, said separating or purifying a sample from the subject comprises contacting the sample with one or more compounds capable of binding an immunoglobulin for a time and under conditions sufficient for binding to occur and isolating the compound.

For example, the one or more compounds is/are previously immobilized on a solid support, matrix or resin, eg., the solid support, matrix or resin is selected from the group consisting of cellulose bead, agarose, nylon, magnetic particle, paramagnetic particle, polymeric resin and mixtures thereof.

The method of the invention additionally provides for washing the one or more immobilized compounds to thereby remove non-specifically bound or unbound protein.

In one example, a compound is selected from the group consisting of protein A or a mimetic thereof, protein G or a mimetic thereof, protein L or a mimetic thereof, an anti-immunoglobulin antibody, a maltose binding protein (MBP) and a thiophilic resin or mixtures thereof. For instance, a compound is Protein A, Protein G or mixtures thereof.

The method of the present invention additionally provides for linking immunoglobulin to the one or more compounds, eg., by performing a process comprising contacting a cross-linking agent with the one or more compounds having immunoglobulin bound thereto for a time and under conditions sufficient for covalent linkage to occur between a compound and immunoglobulin.

Such a cross-linking agent is, for example, selected from the group consisting of an imidoester cross-linker, a N-hydroxysuccinimide cross-linker, a maleimide cross-linker, a haloacetyl cross-linker, a hydrazide cross-linker, and a carbodiimide cross-linker. For instance, the cross linking agent is a N-hydroxysuccinimide cross-linker, eg., the N-hydroxysuccinimide cross-linker is selected from the group consisting of disuccinimidyl glutarate, disuccinimidyl suberate, bis(sulfosuccinimidyl)suberate, dithiobis(succinimidyl propionate), 3,3′-dithiobis(succinimidyl propionate), ethylene glycobis(succinimidyl succinate), ethylene glycobis(sulfo-succinimidylsuccinate), disuccinimidyl tartarate, disulfosuccinimidyl tartarate, bis[2-(succinimidyloxy-carbonyloxy) ethyl]sulfone, bis[2-(sulfosuccinimidyloxy-carbonyloxy)ethyl]sulfone, succinimidyl 4-(N-maleimidomethyl)cyclohexane-1-carboxylate, sulfo-succinimidyl 4-(N-maleimidomethyl)cyclohexane-1-carboxylate, m-maleimido benzoyl-N-hydroxysuccinimide ester, m-maleimido benzoyl-N-hydroxysulfo succinimide ester, succinimidyl 4-(p-maleimidophenyl)-butyrate, sulfo-succinimidyl 4-(p-maleimidophenyl)-butyrate, bismaleimidohexane, N-(g-maleimidobutyryloxy)succinimide ester and N-(g-maleimidobutyryloxy) sulfosuccinimide ester. For example, the N-hydroxysuccinimide cross-linker is disuccinimidyl suberate.

The invention also provides a method for identifying an immunogenic protein or fragment thereof from an autoimmune condition capable of eliciting an immune response subject, said method comprising:
(i) obtaining a protein complex comprising an immunoglobulin or mixtures thereof or an immunoglobulin-containing fraction from a subject suffering from an autoimmune condition or a cell, tissue or organ thereof:
(ii) contacting immunoglobulin in the protein complex or immunoglobulin-containing fraction with a sample comprising protein from a subject suffering from an autoimmune disease; and
(ii) identifying a protein or fragment thereof bound to said immunoglobulin by virtue of an antigen-antibody interaction,
wherein the identified protein is an immunogenic protein or fragment thereof from an autoimmune condition capable of eliciting an immune response in a human or non-human animal subject

In one instance, the invention further comprises obtaining a sample that comprises the protein complex or immunoglobulin-containing fraction from the subject suffering from an autoimmune disease and e.g., obtaining an immunoglobulin or mixtures thereof or an immunoglobulin-containing fraction thereof from the sample. Additionally, the method additionally comprises, for example, obtaining an immunoglobulin-containing fraction from the sample.

For example, the sample is selected from the group consisting of whole blood, plasma, serum, sputum, saliva, pleural fluid, pericardial fluid, peritoneal fluid, lymph fluid, lymph node, spleen, egg yolk, a fraction of whole blood, a fraction of plasma, a fraction of serum, a fraction of sputum, a fraction of saliva, a fraction of pleural fluid, a fraction of pericardial fluid, a fraction of peritoneal fluid, a fraction of lymph fluid, a fraction of lymph node, a fraction of spleen and a fraction of egg yolk. In another example, the sample comprises a cell selected from the group consisting of peripheral blood mononuclear cell (PBMC), lymphocyte, B-lymphocyte, T lymphocyte, helper T-cell, cytotoxic T cell, macrophage, dendritic cell, polymorphonuclear cell and mast cell. In yet another example, the sample comprises serum or an immunoglobulin-containing fraction of serum.

In one example, the protein complex or immunoglobulin-containing fraction comprises one or more immunoglobulins selected from the group consisting of IgM, IgG, IgA, IgE, IgD and IgY or mixtures thereof. For example, the protein complex or immunoglobulin-containing fraction comprises IgG. Alternatively, or in addition, the protein complex or immunoglobulin-containing fraction comprises IgA. Alternatively, or in addition, the protein complex or immunoglobulin-containing fraction comprises IgM.

The invention provides for obtaining the protein complex or immunoglobulin-containing fraction by a process comprising separating or purifying a sample from the subject to thereby provide said protein complex or immunoglobulin-containing fraction. For example, said separating or purifying a sample from the subject comprises contacting the sample with one or more compounds capable of binding an immunoglobulin for a time and under conditions sufficient for binding to occur and isolating the compound.

For example, the compound is previously immobilized on a solid support, matrix or resin, eg., the solid support, matrix or resin is selected from the group consisting of cellulose bead, agarose, nylon, magnetic particle, paramagnetic particle and polymeric resin and mixtures thereof.

The method of the invention additionally provides for washing the one or more immobilized compounds to thereby remove non-specifically bound or unbound protein.

In one example, the compound is selected from the group consisting of Protein A or a mimetic thereof, Protein G or a mimetic thereof, Protein L or a mimetic thereof, an anti-immunoglobulin antibody, a maltose binding protein (MBP), a thiophilic resin and mixtures thereof. For instance, the compound is Protein A, Protein G or mixtures thereof.

The method of the present invention additionally provides for linking immunoglobulin to the one or more compounds, eg., by performing a process that comprises contacting a cross-inking agent with the one or more compounds having immunoglobulin bound thereto for a time and under conditions sufficient for covalent linkage to occur between a compound and immunoglobulin.

Such a cross-linking agent is, for example, selected from the group consisting of an imidoester cross-linker, a N-hydroxysuccinimide cross-linker, a maleimide cross-linker, a haloacetyl cross-linker, a hydrazide cross-linker, and a carbodiimide cross-linker. For instance, the cross linking agent is a N-hydroxysuccinimide cross-linker, eg., the N-hydroxysuccinimide cross-linker is selected from the group consisting of disuccinimidyl glutarate, disuccinimidyl suberate, bis(sulfosuccinimidyl)suberate, dithiobis(succinimidyl propionate), 3,3′-dithiobis(succinimidyl propionate), ethylene glycobis(succinimidyl succinate), ethylene glycobis(sulfo-succinimidylsuccinate), disuccinimidyl tartarate, disulfosuccinimidyl tartarate, bis[2-(succinimidyloxy-carbonyloxy)ethyl]sulfone, bis[2-(sulfosuccinimidyloxy-carbonyloxy)ethyl]sulfone, succinimidyl 4-(p-maleimidomethyl)cyclohexane-1-carboxylate, sulfo-succinimidyl 4-(N-maleimidomethyl)cyclohexane-1-carboxylate, m-maleimido benzoyl-N-hydroxysuccinimide ester, m-maleimido benzoyl-N-hydroxysulfo succinimide ester, succinimidyl 4-(p-maleimidophenyl)-butyrate, sulfo-succinimidyl 4-(p-maleimidophenyl)-butyrate, bismaleimidohexane, N-(g-maleimidobutyryloxy)succinimide ester and N-(g-maleimidobutyryloxy) sulfosuccinimide ester. For example, the N-hydroxysuccinimide cross-linker is disuccinimidyl suberate.

In an example, the subject is a human and suffers from an autoimmune condition. For example, the autoimmune condition is an autoimmune disease, such as, for example, an autoimmune disease is selected from the group consisting of rheumatoid arthritis, multiple sclerosis, type-1 diabetes, inflammatory bowel disease, Crohn's Disease, ulcerative colitis, systemic lupus erythematosus, psoriasis, scleroderma, autoimmune thyroid disease, central nervous system vasculitis, and autoimmune myositis.

In another example, the subject suffers from cystic fibrosis (CF). In one example, such a CF subject has previously suffered from an acute pulmonary exacerbation In another example, the CF subject is suffering from an acute pulmonary exacerbation. In yet another example, the subject additionally suffers from an infection, eg., an infection caused by a bacterium. Such an infection is caused, for example, by a bacterium selected from the group consisting of Staphylococcus aureus, Pseudomonas aeruginosa, Haemophilus influenzae, Aspergillus fumigatus, Burkholderia cepacia complex, Stenotrophomonas maltophila, Alcaligenes (Achromobacter) xylosoxidans, B. gladioli and Ralstonia picketti or mixtures thereof, eg., the bacterial infection comprises a Pseudomonas aeruginosa infection.

In an example of the method of the present invention, the sample is derived from the subject from which the protein complex comprising an immunoglobulin or mixtures thereof or an immunoglobulin-containing fraction thereof was derived.

The invention additionally provides, a method for identifying an immunogenic protein or fragment thereof capable of eliciting an immune response in a subject, said method comprising:
(i) obtaining a protein complex comprising an immunoglobulin or mixtures thereof or an immunoglobulin-containing fraction from a sample from or produced by a subject previously administered with a sample comprising a cell or cell extract or mixture thereof comprising the immunogenic protein or fragment thereof;
(ii) contacting the protein complex or immunoglobulin-containing fraction with a sample comprising the cell or cell extract or mixture thereof; and
(iii) identifying a protein or fragment thereof bound to immunoglobulin in the protein complex or immunoglobulin-containing fraction by virtue of an antigen antibody interaction,
thereby identifying an immunogenic protein or fragment thereof capable of eliciting an immune response in a subject.

In one example, the method further comprises administering sample comprising the cell or cell extract to the subject. The cell or cell extract is, for example, derived from an agent that causes a disease or disorder.

An agent that causes a disease or disorder is, for example, an infectious agent, eg., an infectious agent selected from the group consisting of a virus, a bacterium, a yeast, a fungus and a parasite. For example, the infectious agent is a bacterium, eg., Mycobacterium tuberculosis, e.g a clinical isolate of M. tuberculosis.

In one instance, the invention further comprises obtaining a sample from or produced by the subject and/or obtaining the protein complex or immunoglobulin-containing fraction. Additionally, the method comprises, for example, obtaining an immunoglobulin-containing fraction from the sample.

In one form of the method the subject is a non-human animal, for example a non-human animal selected from the group consisting of a mouse, a rat, a rabbit, a chicken, a dog, a sheep, an ovine, a horse, a donkey and a goat. In one exemplified form of the invention the subject is avian (eg. a chicken) and the biological sample produced by the subject is an egg or an extract thereof or a derivative thereof.

In another example, the sample is selected from the group consisting of whole blood, plasma, serum, sputum, saliva, pleural fluid, pericardial fluid, peritoneal fluid, lymph fluid, lymph node, spleen, egg yolk, a fraction of whole blood, a fraction of plasma, a fraction of serum, a fraction of sputum, a fraction of saliva, a fraction of pleural fluid, a fraction of pericardial fluid, a fraction of peritoneal fluid, a fraction of lymph fluid, a fraction of lymph node, a fraction of spleen and a fraction of egg yolk. In another example, the sample comprises a cell selected from the group consisting of peripheral blood mononuclear cell (PBMC), lymphocyte, B-lymphocyte, T lymphocyte, helper T-cell, cytotoxic T cell, macrophage, dendritic cell, polymorphonuclear cell and mast cell. In yet another example, the sample comprises serum or an immunoglobulin-containing fraction of serum or egg yolk or an immunoglobulin-containing fraction of egg yolk.

In one example, the protein complex or immunoglobulin-containing fraction comprises one or more immunoglobulins selected from the group consisting of IgM, IgG, IgA, IgE, IgD and IgY or mixtures thereof. For example, the protein complex or immunoglobulin-containing fraction comprises IgG. Alternatively, or in addition, the protein complex or immunoglobulin-containing fraction comprises IgA. Alternatively, or in addition, the protein complex or immunoglobulin containing fraction or protein complex comprises IgY. Alternatively, or in addition, the protein complex or immunoglobulin-containing fraction comprises IgM.

The invention provides for obtaining the protein complex or immunoglobulin-containing fraction by a process comprising separating or purifying a sample from the subject to thereby provide said protein complex or immunoglobulin-containing fraction. For example, said separating or purifying a sample from the subject comprises contacting the sample with one or more compounds capable of binding an immunoglobulin for a time and under conditions sufficient for binding to occur and isolating the compound.

For example, the compound is previously immobilized on a solid support, matrix or resin, eg., the solid support, matrix or resin is selected from the group consisting of cellulose bead, agarose, nylon, magnetic particle, paramagnetic particle, polymeric resin and mixtures thereof.

The method of the invention additionally provides for washing the immobilized compound to thereby remove non-specifically bound or unbound protein.

In one example, the one or more compounds is selected from the group consisting of Protein A or a mimetic thereof, Protein G or a mimetic thereof, Protein L or a mimetic thereof, an anti-immunoglobulin antibody, a maltose binding protein (MBP), a thiophilic resin and mixtures thereof. For instance, the compound is Protein A or Protein G or mixtures thereof.

The method of the present invention additionally provides for linking immunoglobulin to the compound, eg., by performing a process that comprises contacting a cross-linking agent with the one or more compounds having immunoglobulin bound thereto for a time and under conditions sufficient for covalent linkage to occur between a compound and immunoglobulin.

Such a cross-linking agent is, for example, selected from the group consisting of an imidoester cross-linker, a N-hydroxysuccinimide cross-linker, a maleimide cross-linker, a haloacetyl cross-linker, a hydrazide cross-linker, and a carbodiimide cross-linker. For instance, the cross linking agent is a N-hydroxysuccinimide cross-linker, eg., the N-hydroxysuccinimide cross-linker is selected from the group consisting of disuccinimidyl glutarate, disuccinimidyl suberate, Bis(sulfosuccinimidyl)suberate, dithiobis(succinimidyl propionate), 3,3′-dithiobis(succinimidyl propionate), ethylene glycobis(succinimidyl succinate), ethylene glycobis(sulfo-succinimidylsuccinate), disuccinimidyl tartarate, disulfosuccinimidyl tartarate, bis[2-(succinimidyloxy-carbonyloxy)ethyl]sulfone, bis[2-(sulfosuccinimidyloxy-carbonyloxy)ethyl]sulfone, succinimidyl 4-(N-maleimidomethyl)cyclohexane-1-carboxylate, sulfo-succinimidyl 4-(N-maleimidomethyl)cyclohexane-1-carboxylate, m-maleimido benzoyl-N-hydroxysuccinimide ester, m-maleimido benzoyl-N-hydroxysulfo succinimide ester, succinimidyl 4-(p-maleimidophenyl)-butyrate, sulfo-succinimidyl 4-(p-maleimidophenyl)-butyrate, bismaleimidohexane, N-(g-maleimidobutyryloxy)succinimide ester and N-(g-maleimidobutyryloxy)sulfosuccinimide ester. For example, the N-hydroxysuccinimide cross-linker is disuccinimidyl suberate.

In one example of the method of the invention, the sample comprising the cell or cell extract or mixture thereof that is contacted to the protein complex or immunoglobulin-containing fraction is derived from a subject comprising the cell or cell extract. For example, the cell or cell extract is derived from an agent that causes a disease or disorder and the sample comprising the cell or cell extract or mixture thereof is derived from a subject suffering from the disease or disorder.

In one example, the agent that causes a disease or disorder is an infectious agent, for example, an infectious agent selected from the group consisting of a virus, a bacterium, a yeast, a fungus and a parasite.

As exemplified herein, the infectious agent is a bacterium, eg., Mycobacterium tuberculosis.

The present invention additionally comprises, for example, separating an immunogenic protein or fragment thereof bound to the immunoglobulin by virtue of an antigen-antibody interaction from the immunoglobulin. Such separation is achieved, for example, by performing a process that comprises contacting the protein complex comprising an immunoglobulin or mixtures thereof or an immunoglobulin-containing fraction thereof with a compound that disrupts the antigen-antibody interaction for a time an under conditions sufficient to disrupt the antigen-antibody interaction.

For example, a compound that disrupts the antigen-antibody interaction is selected from the group consisting a compound that modulates the pH of the immunoglobulin-containing fraction, a salt, an ionic detergent, a dissociating agent and a chaotropic agent or mixtures thereof.

In another example, the present invention additionally comprises isolating a protein that is or was bound to the immunoglobulin-containing fraction by virtue of an antigen-antibody interaction. For example, the protein is isolated using gel electrophoresis, eg., two-dimensional gel electrophoresis.

In yet another example, a protein that is or was bound to the immunoglobulin-containing fraction by virtue of an antigen-antibody interaction is identified using mass spectrometry, eg., matrix-assisted laser desorption/ionisation-time-of-flight mass spectrometry (MALDI-TOF MS).

The present invention additionally provides a method comprising:
(a) obtaining a protein complex comprising an immunoglobulin or mixtures thereof or an immunoglobulin-containing fraction from a subject that has raised an immune response against an immunogenic protein or fragment thereof or a cell, tissue or organ thereof by a method comprising contacting a sample from the subject with one or more compounds capable of binding an immunoglobulin for a time and under conditions sufficient for binding to occur and isolating the one or more compounds;
(b) linking immunoglobulin in the protein complex or immunoglobulin-containing fraction to the one or more compounds;
(c) separating an immunogenic protein or fragment thereof from the linked immunoglobin;
(d) contacting a sample comprising the immunogenic protein or fragment thereof with the linked immunoglobulin;
(e) separating the immunogenic protein or fragment thereof from the linked immunoglobulin;
(f) optionally, repeating (d) and (e) one or more times; and
(g) identifying a protein or fragment thereof separated from the immunoglobulin,
thereby identifying an immunogenic protein or fragment thereof.

In one form of the method (e) separating the immunogenic protein or fragment thereof from the linked immunoglobulin is performed prior to (d) contacting a sample comprising the immunogenic protein or fragment thereof with the linked immunoglobulin.

In another form of the invention (d) contacting a sample comprising the immunogenic protein or fragment thereof with the linked immunoglobulin is performed prior to (e) separating the immunogenic protein or fragment thereof from the linked immunoglobulin.

Preferably, (d) contacting a sample comprising the immunogenic protein or fragment thereof with the linked immunoglobulin and (e) separating the immunogenic protein or fragment thereof from the linked immunoglobulin are repeated a sufficient number of times to identify one or more immunogenic proteins. For example, (d) contacting a sample comprising the immunogenic protein or fragment thereof with the linked immunoglobulin and (e) separating the immunogenic protein or fragment thereof from the linked immunoglobulin are repeated a sufficient number of times to distinguish one or more proteins or fragments thereof on a gel using gel electrophoresis, for example, two-dimensional gel electrophoresis.

In one example of the method, the subject has raised an immune response against an agent that causes a disease or disorder. In accordance with this example, the sample comprising the immunogenic protein or fragment thereof that is contacted with the linked immunoglobulin comprises the agent that causes the disease or disorder or a derivative thereof.

For example, the agent that causes the disease or disorder is an infectious agent, eg., a bacterium, eg., Mycobacterium tuberculosis.

In another example, the subject suffers from an autoimmune condition. In accordance with this example, the sample comprising the immunogenic protein or fragment thereof that is contacted with the linked immunoglobulin comprises protein from a subject suffering from an autoimmune condition.

In yet another example, the subject has been previously immunized with a sample comprising a cell or extract thereof or mixtures thereof comprising the immunogenic protein or fragment thereof. In accordance with this example the sample comprising the immunogenic protein or fragment thereof that is contacted with the linked immunoglobulin comprises the cell or extract thereof. In one form, the subject is a chicken.

In an exemplified form of the method the subject has been previously immunized with a cell or cell extract from an agent associated with a disease or disorder, eg., an infectious agent, eg., a bacterium. In one example, the bacterium is Mycobacterium tuberculosis.

The invention also provides for the use of the method described herein in a process for identifying a marker of a condition.

The invention additionally provides for the use of the method described herein in the diagnosis of a condition.

In one example, the condition is a disease or disorder, for example an infectious disease or a cancer.

In another example, the condition is an autoimmune condition.

### BRIEF DESCRIPTION OF THE DRAWINGS

FIG. 1 is a photographic representation of a 2-dimensional gel showing proteins that have been isolated with an immunoglobulin fraction of a subject suffering from M. tuberculosis.

FIG. 2 is a photographic representation of a 2-dimensional gel showing proteins that have been isolated with an immunological fraction using the method of the present invention. A protein of interest designated Protein Spot 3A is highlighted.

FIG. 3 is a graphical representation showing the mass spectrum of a tryptic peptide from the protein identified in FIG. 1.

FIG. 4 is a photographic representation of a 2-dimensional gel showing proteins that have been captured from P. aeruginosa using an immunoglobulin-containing fraction from a CF subject suffering from a P. aeruginosa infection.

FIG. 5 is a photographic representation showing a 2-dimensional gel showing proteins captured from sputum of a CF subject using an immunoglobulin-containing fraction from a CF subject that is suffering from an exacerbated state and a P. aeruginosa infection.

FIG. 6 is a photographic representation showing immunoreactivity of four CF subjects and three healthy control subjects to P. aeruginosa GroES. Each spot position in the 4- or 5-spot containing grid shows the immunoreactivity of a single subject to the protein onto which plasma aliquots were analysed. Spot positions 1 to 3 are from healthy control subjects. Spot positions 4 to 7 are from CF subjects. Also shown in the specificity of observed immunoreactivities by screening patient samples against negative controls (BSA or PBS).

FIG. 7 is a photographic representation showing a two-dimensional gel showing proteins isolated from sputum of a subject suffering from tuberculosis using an immunoglobulin-containing fraction obtained from an egg layed by a chicken previously immunized with a cellular extract of Mycobacterium tuberculosis.

### DETAILED DESCRIPTION OF THE PREFERRED EMBODIMENTS

The present invention provides a method for identifying an immunogenic protein or fragment thereof capable of eliciting an immune response, said method comprising obtaining a protein complex comprising an immunoglobulin or mixtures thereof or an immunoglobulin-containing fraction from a subject or a cell, tissue or organ thereof and identifying a protein or fragment thereof bound to the immunoglobulin by virtue of an antigen antibody interaction, thereby identifying an immunogenic protein or fragment thereof capable of eliciting an immune response.

As used herein the term “immunogenic protein” shall be understood to mean any peptide, polypeptide or protein that induces an immune response in a subject, such that a specific antibody is raised against said protein by the subject. Accordingly, such an “immunogenic protein” is capable of interacting with an antibody as an antigen, ie., an antibody is capable of binding to the immunogenic protein in a non-covalent manner by virtue of an interaction with the hypervariable or complementarity determining region of the antibody.

A protein or fragment that is capable of binding to an immunoglobulin by virtue of an antigen-antibody interaction does so by any one or more of a variety of bonds or attractive forces. For example, an antigen binds to an antibody by virtue of hydrogen bonding (ie. the formation of hydrogen bridges between appropriate molecules of the antibody and the antigen), Van der Waals bonds (interaction between electron clouds, ie. oscillating dipoles) or hydrophobic bonds, which rely upon the association of non-polar, hydrophobic groups. Alternatively, or in addition, an antigen-antibody interaction is facilitated by electrostatic forces, it., the interaction of oppositely charged groups on protein side chains.

In one example, the method for isolating and/or identifying an immunogenic protein isolated with an immunoglobulin of the present invention isolates a native or unmodified form of an immunogenic protein. As used herein, the term “native or unmodified form” shall be taken to mean a form of a protein that is found in nature. Accordingly, this term encompasses modifications of said protein that are found in the environment in which the protein is found in nature, for example, in a cell in which the protein is naturally expressed. Such modifications include, for example, proteolytic cleavage, post-translational modification, alternative splice forms, and any other modifications result in a change in molecular weight, charge, or amino acid composition of the protein.

In another example, a method for isolating and/or identifying an immunogenic protein isolated with an immunoglobulin-containing fraction of the present invention isolates a modified form, a fragment or a peptide of an immunogenic protein. As used herein the term “modified form” shall be understood to mean a protein that differs to the native form of said protein. Modifications that may be detected by such methods include, for example, proteolytic cleavage, post-translational modification, and any other modifications result in a change in molecular weight, charge, or amino acid composition. In accordance with this embodiment, a modified form, a fragment or a peptide of an immunogenic protein may be produced as a result of an immune response by a subject from whom a biological sample is derived. During such an immune response, many proteolytic enzymes, such as, for example neutrophil elastase and pathogen derived elastases are produced, often resulting in the cleavage and degradation of a protein. Accordingly, a modified form of an immunogenic protein that is clearly encompassed by the present invention is a fragment of said protein. As a consequence, the present invention clearly encompasses the isolation of a fragment of an immunogenic protein or an immunogenic fragment of a protein.

A preferred fragment includes a fragment of a protein against which an individual mounts a specific immune response, more preferably, a specific antibody response. Preferably, a fragment of protein identified by the method of the present invention comprises at least about 5 amino acids, more preferably at least about 6 amino acids, more preferably, about 10 amino acids, even more preferably, about 20 amino acids, even more preferably, about 50 amino acids and even more preferably, about 100 amino acids.

As used herein, the term “eliciting an immune response” shall be understood to refer to the ability of a subject to raise a specific antibody response and/or a specific T-cell response to an antigen. Preferably, the immune response is an antibody response. Without wishing to be being bound by theory or mode of action, such an antibody response it expected to comprise a B-lymphocyte (or cell) producing IgD and IgM that specifically bind to the immunogenic protein or antigen. It is particularly preferred that said B-lymphocyte then differentiates into a plasma cell that secretes IgM and/or IgG and/or IgE and/or IgA and/or IgY that specifically binds to said immunogenic protein.

As used herein, the term “protein complex comprising an immunoglobulin” shall be taken to mean a plurality of interacting proteins that comprise one or more immunoglobulin proteins and/or one or more types of immunoglobulin protein. In one embodiment, such a protein complex is an antibody complex, eg., a number of immunoglobulin molecules that are linked in a covalent manner (eg. by disulphide bonding) to form an antibody. For example, in the case of IgG such an antibody complex comprises two light chains and two heavy chains, wherein each light chain is linked to a heavy chain by at least one disulphide bond and the heavy chains are also linked by at least one disulphide bond. Clearly other forms of antibody complex are contemplated b the present invention, for example, the pentameric structure of IgM. Furthermore, in one embodiment, such an antibody complex comprises an antigen bound to an immunoglobulin. Clearly a protein complex includes an immunogenic protein or fragment thereof bound to one or more immunoglobulin by virtue of an antigen-antibody interaction

As used herein the term “immunoglobulin-containing fraction” shall be taken to mean a component of a biological sample that is isolated with an immunoglobulin. Such a fraction may comprise, for example, an immune complex, an antibody-HLA complex, an antibody, immunoglobulin light chain, immunoglobulin heavy chain, a component of the complement pathway, fibrinogen, haptoglobin, α-1-antitrypsin, α-1-acid glycoprotein, α-1-macroglobulin, transferrin, low density lipoprotein, ceruloplasmin or serum albumin protein or a fragment thereof bound to immunoglobulin by virtue of an antigen-antibody interaction or mixtures thereof amongst other components.

In a preferred embodiment, an “immunoglobulin-containing fraction” and/or a “protein complex comprising an immunoglobulin or a mixture thereof” is to be considered those proteins that are directly bound by an immunoglobulin binding compound, such as, for example, Protein G, Protein A or Protein L.

As used herein the term “immunoglobulin” shall be taken to mean a protein produced by lymphocytes, where said protein preferably has specific antibody activity, ie., it is capable of interacting with/binding to a specific protein, preferably without formation of a covalent bond. Preferably an immunoglobulin comprises four polypeptide chains, two identical heavy chains and two identical light chains, linked by disulphide bonds. It is preferred that an immunoglobulin is selected from the group consisting of IgA, IgD, IgE, IgG, IgM and IgY.

As used herein the term “antibody” refers to intact monoclonal or polyclonal antibodies, immunoglobulin (IgA, Ig, IgG, IgM, IgE, IgY) fractions, humanized antibodies, or recombinant single chain antibodies, as well as fragments thereof, such as, for example Fab, F(ab)2, and Fv fragments.

In one example of the invention, the protein complex or immunoglobulin-containing fractions comprises IgG and/or IgA and/or IgY. In another example of the invention, the protein complex or immunoglobulin-containing fraction comprises IgG. In yet another example of the invention, the immunoglobulin-containing fraction comprises IgA. In a further example of the invention, the protein complex or immunoglobulin-containing fraction comprises IgY. In a still further example of the invention the protein complex or immunoglobulin-containing fraction comprises IgM.

The present invention provides for the isolation of an immunoglobulin-containing fraction or a component of an immunoglobulin-containing fraction. Preferably, this process is performed without purifying free immunoglobulin from immunoglobulin that is complexed with an antigen or immunogenic protein.

Biological Samples

As immunoglobulin is found within various body tissues and/or body fluids, the present invention clearly encompasses obtaining a biological sample that comprises the protein complex or immunoglobulin-containing fraction from a sample comprising such a body tissue or body fluid. An example of a biological sample useful for the isolation of the protein complex or mixture thereof or immunoglobulin-containing fraction is a sample selected from the group consisting of whole blood, plasma, serum, sputum, saliva, pleural fluid, pericardial fluid, peritoneal fluid, lymph fluid, lymph node, spleen, egg yolk, a fraction of whole blood, a fraction of plasma, a fraction of serum, a fraction of sputum, a fraction of saliva, a fraction of pleural fluid, a fraction of pericardial fluid, a fraction of peritoneal fluid, a fraction of lymph fluid, a fraction of lymph node, a fraction of spleen and a fraction of egg yolk.

Those biological samples useful for the performance of the present invention comprise, for example, a cell selected from the group consisting of peripheral blood mononuclear cell (PBMC), lymphocyte, B-lymphocyte, T lymphocyte, helper T-cell, cytotoxic T cell, macrophage, dendritic cell, polymorphonuclear cell and mast cell. For example, such a biological sample comprises serum or an immunoglobulin-containing fraction of serum.

Clearly the present invention also encompasses derivatives of said biological samples. For example, the present invention encompasses samples that have been treated to isolate one or more proteins from a biological sample (for example to remove protein below a predetermined molecular weight, or to remove a common protein, such as, for example, albumin). Alternatively, or in addition, a biological sample, such as, for example, whole blood is treated to cause clotting of erythrocytes to facilitate isolation of plasma or serum.

As the present invention encompasses obtaining a biological sample, it will be apparent that, in some forms of the invention, a biological sample is derived previously from a subject using a method known in the art, such as, for example using a syringe or by surgery.

The method of the present invention encompasses biological samples derived from any subject that is capable of eliciting an immune response against an antigen. For example, such a subject is a non-human animal, such as, for example, a drosophila, a Caenorhabditis elegans, a zebrafish, a mouse, a rat, a rabbit, a chicken, a dog, a sheep, an ovine, a horse or a goat. Clearly, developmental stages of these animals is also encompassed by the present invention, for example, as exemplified herein, injection of a cellular extract a chicken causes a specific immune response against specific antigens in that cell extract and immunoglobulin that binds to an antigen in the extract is isolated from an egg laid by said chicken.

Alternatively, the method of the present invention is equally useful for identifying an immunogenic protein or a fragment thereof in a biological sample from a human. Such a method is useful for identifying a protein or fragment thereof in a human that suffers from, for example, an infection or an autoimmune disease, wherein said protein or fragment thereof is a diagnostic and/or therapeutic target.

Alternatively, or in addition, as immunoglobulin that specifically binds to a protein or a fragment thereof exists in or is produced by a subject following an infection, an immunoglobulin-containing fraction from a subject previously infected or currently infected is useful for identifying an immunogenic protein from the infectious agent.

A biological sample derived from subject suffering from an infection or previously infected with an infectious agent is useful for, for example, identifying a marker and/or therapeutic target of said infection. Such an infection is, for example, an acute infection or a chromic infection. By “chronic” is meant that an infection is long lasting or characterised by frequent recurrences. For example, a chronic bacterial infection is often observed in a subject suffering from cystic fibrosis.

By “acute” is meant that an infection has a rapid onset followed by a short and possibly severe course. For example, subjects suffering from a hepatitis C infection or a Hemophilus influenzae often suffer from an acute infection.

A biological sample derived from a subject suffering from an infection or previously suffering from an infection (whether acute or chronic) selected from the group consisting of a viral infection, a bacterial infection, a yeast infection, a fungal infection and a parasitological infection is encompassed by the present invention.

For example, a biological sample is derived from a subject suffering from (or previously suffering from) a bacterial infection, wherein the bacteria that is causative of said bacterial infection is from a genus selected from the group consisting of Abiotrophia, Achromobacter, Acidaminococcus, Acidovorax, Acinetobacter, Actinobacillus, Actinobaculum, Actinomadura, Actinomyces, Aerococcus, Aeromonas, Afipia, Agrobacterium, Alcaligenes, Alloiococcus AlteromonasAmycolata, Amycolatopsis, Anaerobospirillum, Anaerorhabdus, “Anguillina”, Arachnia, Arcanobacterium, Arcobacter, Arthrobacter, Atopobium, Aureobacterium, Bacillus, Bacteroides, Balneatrix, Bartonella, Bergeyella, Bifidobacterium, Bilophila, Branhamella, Borrelia, Bordetella, Brachyspira, Brevibacillus, Brevibacterium, Brevundimonas, Brucella, Burkholderia, Buttiauxella, Butyrivibrio, Calymmatobacterium, Campylobacter, Capnocytophaga, Cardiobacterium, Catonella, Cedecea, Cellulomonas, Centipeda, Chlamydia, Chlamydophila, Chromobacterium, Chyseobacterium, Chryseomonas, Citrobacter, Clostridium, Collinsella, Comamonas, Corynebacterium, Coxiella, Cryptobacterium, Delftia, Dermabacter, Dermatophilus, Desulfomonas, Desulfovibrio, Dialister, Dichelobacter, Dolosicoccus, Dolosigranulum, Edwardsiella, Eggerthella, Ehrlichia, Eikenella, Empedobacter, Enterobacter, Enterococcus, Erwinia, Erysipelothrix, Escherichia, Eubacterium, Ewingella, Exiguobacterium, Facklamia, Filifactor, Flavimonas, Flavobacterium, Flexispira, Francisella, Fusobacterium, Gardnerella, Gemella Globicatella, Gordona, Haemophilus, Hafnia, Helicobacter, Helococcus, Holdemania, Ignavigranum, Johnsonella, Kingella, Klebsiella, Kocuria, Koserella, Kurthia, Kytococcus, Lactobacillus, Lactococcus, Lautropia, Leclercia, Legionella, Leminorella, Leptospira, Leptotrichia, Leuconostoc, Listeria, Listonella, Megasphaera, Methylobacterium, Microbacterium, Micrococcus, Mitsuokella, Mobiluncus, Moellerella, Moraxella, Morganella, Mycobacterium, Mycoplasma, Myroides, Neisseria, Nocardia, Nocardiopsis, Ochrobactrum, OeskoviaOligella, Orientia, Paenibacillus, Pantoea, Parachlamydia, Pasteurella, Pediococcus, Peptococcus, Peptostreptococcus, Photobacterium, Photorhabdus, Plesiomonas Porphyrimonas, Prevotella, Propionibacterium, Proteus, Providencia, Pseudomonas, Pseudonocardia, Pseudoramibacter, Psychrobacter, Rahnella, Ralstonia, Rhodococcus, Rickettsia, Rochalimaea, Roseomonas, Rothia, Ruminococcus, Salmonella, Selenomonas, Serpulina, Serratia, Shewenella, Shigella, Simkania, Slackia, Sphingobacterium, Sphingomonas, Spirillum, Staphylococcus, Stenotrophomonas, Stomatococcus, Streptobacillus, Streptococcus, Streptomyces, Succinivibrio, Sutterella, Suttonella, Tatumella, Tissierella, Trabulsiella, Treponema, Tropheryma, Tsakamurella, Turicella, Ureaplasma, Vagococcus, Veillonella, Vibrio, Weeksella, Wolinella, Xanthomonas, Xenorhabdus, Yersinia and Yokenella.

For example, a bacterial infection is caused by a bacterium selected from the group consisting of Actimomyces europeus, Actimomyces georgiae, Actimomyces gerencseriae, Actimomyces graevenitzii, Actimomyces israelii, Actimomyces meyeri, Actimomyces naeslundii, Actimomyces neuii neuii, Actimomyces neuii anitratus, Actimomyces odontolyticus, Actimomyces radingae, Actimomyces turicensis, Actimomyces viscosus, Arthrobacter creatinolyticus, Arthrobacter cumminsii, Arthrobacter woluwensis, Bacillus anthracis, Bacillus cereus, Bacillus circulans, Bacillus coagulans, Bacillus licheniformis, Bacillus megaterium, Bacillus myroides, Bacillus pumilus, Bacillus sphaericus, Bacillus subtilis, Bacillus thuringiensis, Borrelia afzelii, Borrelia andersonii, Borrelia bissettii, Borrelia burgdorferi, Borrelia garinii, Borrelia japonica, Borrelia lusitaniae, Borrelia tanukii, Borrelia turdi, Borrelia valaisiana Borrelia caucasica, Borrelia crocidurae, Borrelia recurrentis, Borrelia duttoni, Borrelia graingeri, Borrelia hermsii, Borrelia hispanica, Borrelia latyschewii, Borrelia mazzottii, Borrelia parkeri, Borrelia persica, Borrelia recurrentis, Borrelia turicatae, Borrelia venezuelensi, Bordetella bronchiseptica, Bordetella hinzii, Bordetella holmseii, Bordetella parapertussis, Bordetella pertussis, Bordetella trematum, Clostridium absonum, Clostridium argentinense, Clostridium baratii, Clostridium bifermentans, Clostridium beijerinckii, Clostridium butyricum, Clostridium cadaveris, Clostridium carnis, Clostridium celatum, Clostridium clostridioforme, Clostridium cochlearium, Clostridium cocleatum, Clostridium fallax, Clostridium ghonii, Clostridium glycolicum, Clostridium haemolyticum, Clostridium hastiforme, Clostridium histolyticum, Clostridium indolis, Clostridium innocuum, Clostridium irregulare, Clostridium leptum, Clostridium limosum, Clostridium malenominatum, Clostridium novyi, Clostridium oroticum, Clostridium paraputrificum, Clostridium piliforme, Clostridium putrefasciens, Clostridium ramosum, Clostridium septicum, Clostridium sordelii, Clostridium sphenoides, Clostridium sporogenes, Clostridium subterminale, Clostridium symbiosum, Clostridium tertium, Escherichia coli, Escherichia fergusonii, Escherichia hermanii, Escherichia vulneris, Enterococcus avium, Enterococcus casseliflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus, Enterococcus solitarius, Haemophilus aegyptius, Haemophilus aphrophilus, Haemophilus paraphrophilus, Haemophilus parainfluenzae, Haemophilus segnis, Haemophilus ducreyi, Haemophilus influenzae, Klebsiella ornitholytica, Klebsiella oxytoca, Klebsiella planticola, Klebsiella pneumoniae, Klebsiella ozaenae, Klebsiella terrigena, Lysteria ivanovii, Lysteria monocytogenes, Mycobacterium abscessus, Mycobacterium africanum, Mycobacterium alvei, Mycobacterium asiaticum, Mycobacterium aurum, Mycobacterium avium, Mycobacterium bohemicum, Mycobacterium bovis, Mycobacterium branderi, Mycobacterium brumae, Mycobacterium celatum, Mycobacterium chelonae, Mycobacterium chubense, Mycobacterium confluentis, Mycobacterium conspicuum, Mycobacterium cookii, Mycobacterium flavescens, Mycobacterium fortuitum, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium interjectum, Mycobacterium heidelberense, Mycobacterium kansasii, Mycobacterium lentiflavum, Mycobacterium leprae, Mycobacterium malmoense, Mycobacterium marinum, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium smegmatis, Mycobacterium szulgai, Mycobacterium terrae, Mycobacterium thermoresistabile, Mycobacterium triplex, Mycobacterium triviale, Mycobacterium tuberculosis, Mycobacterium tusciae, Mycobacterium ulcerans, Mycobacterium vaccae, Mycobacterium wolinskyi, Mycobacterium xenopi, Mycoplasma buccale, Mycoplasma faucium, Mycoplasma fermentans, Mycoplasma genitalium, Mycoplasma hominis, Mycoplasma lipophilum, Mycoplasma orale, Mycoplasma penetrans, Mycoplasma pirum, Mycoplasma pneumoniae, Mycoplasma primatum, Mycoplasma salivarium, Mycoplasma spermatophilum, Pseudomonas aeruginosa, Pseudomonas alcaligenes, Pseudomonas chlororaphis, Pseudomonas fluorescens, Pseudomonas luteola. Pseudomonas mendocina, Pseudomonas monteilii, Pseudomonas oryzihabitans, Pseudomonas pertocinogena, Pseudomonas pseudalcaligenes, Pseudomonas putida, Pseudomonas stutzeri, Rickettsia africae, Rickettsia akari, Rickettsia australis, Rickettsia conorii, Rickettsia felis, Rickettsia honei, Rickettsia japonica, “Rickettsia mongolotimonae,” Rickettsia prowazekii, Rickettsia rickettsiae, Rickettsia sibirica, Rickettsia slovaca, Rickettsia typhi, Salmonella choleraesuis choleraesuis, Salmonella choleraesuis arizonae, Salmonella choleraesuis bongori, Salmonella choleraesuis diarizonae, Salmonella choleraesuis houtenae, Salmonella choleraesuis indica, Salmonella choleraesuis salamae, Salmonella enteritidis, Salmonella typhi, Salmonella typhimurium, Shigella boydii, Shigella dysentaeriae, Shigella flexneri, Shigella sonnei, Staphylococcus aureus, Staphylococcus auricularis, Staphylococcus capitis capitis, Staphylococcus c. ureolyticus, Staphylococcus caprae, Staphylococcus aureus, Staphylococcus cohnii cohnii, Staphylococcus c. urealyticus, Staphylococcus epidermidis, Staphylococcus equorum, Staphylococcus gallinarum, Staphylococcus haemolyticus, Staphylococcus hominis hominis, Staphylococcus h. novobiosepticius, Staphylococcus hyicus, Staphylococcus intermedius, Staphylococcus lugdunensis, Staphylococcus pasteuri, Staphylococcus saccharolyticus, Staphylococcus saprophyticus, Staphylococcus schleiferi schleiferi, Staphylococcus s. coagulans, Staphylococcus sciuri, Staphylococcus simulans, Staphylococcus warneri, Staphylococcus xylosus, Streptococcus agalactiae, Streptococcus canis, Streptococcus dysgalactiae dysgalactiae, Streptococcus dysgalactiae equisimilis, Streptococcus equi equi, Streptococcus equi zooepidemicus, Streptococcus iniae, Streptococcus porcinus, Streptococcus pyogenes, Streptococcus anginosus, Streptococcus constellatus constellatus, Streptococcus constellatus pharyngidis, Streptococcus intermedius, Streptococcus mitis, Streptococcus oralis, Streptococcus sanguinis, Streptococcus cristatus, Streptococcus gordonii, Streptococcus parasanguinis, Streptococcus salivarius, Streptococcus vestibularis, Streptococcus criceti, Streptococcus mutans, Streptococcus ratti, Streptococcus sobrinus, Streptococcus acidominimus, Streptococcus bovis, Streptococcus equinus, Streptococcus pneumoniae, Streptococcus suis, Vibrio alginolyticus, V, carchariae, Vibrio cholerae, C. cincinnatiensis, Vibrio damsela, Vibrio fluvialis, Vibrio furnissii, Vibrio hollisae, Vibrio metschnikovii, Vibrio mimicus, Vibrio parahaemolyticus, Vibrio vulnificus, Yersinia pestis, Yersinia aldovae, Yersinia bercovieri, Yersinia enterocolitica, Yersinia frederiksenii, Yersinia intermedia, Yersinia kristensenii, Yersinia mollaretii, Yersinia pseudotuberculosis and Yersinia rohdei.

As exemplified herein, the method of the present invention is useful for isolating an immunogenic protein from Pseudomonas aeruginosa or Mycobacterium tuberculosis using an immunoglobulin-containing fraction derived from a subject suffering from (and/or that has previously suffered from) said infection.

Alternatively, an immunoglobulin containing fraction derived from a biological sample that is/was derived from a subject suffering from (or previously suffering from) a viral infection is useful for determining an immunogenic protein from said virus. For example, the subject may be suffering from a viral infection, for example, by a virus from a family selected from the group consisting of Astroviridae, Caliciviridae, Picornaviridae, Togaviridae, Flaviviridae, Caronaviridae, Paramyxviridae, Orthomyxoviridae, Bunyaviridae, Arenaviridae, Rhabdoviridae, Filoviridae, Reoviridae, Bornaviridae, Retroviridae, Poxviridae, Herpesviridae, Adenoviridae, Papovaviridae, Parvoviridae, Hepadnaviridae, (eg., a virus selected from the group consisting of a Coxsackie A-24 virus Adenovirus 11, Adenovirus 21, Coxsackie B virus, Borna Disease Virus, Respiratory syncytial virus, Parainfluenza virus, California encephalitis virus, human papilloma virus, varicella zoster virus, Colorado tick fever virus, Herpes Simplex Virus, vaccinia virus, parainfluenza virus 1, parainfluenza virus 2, parainfluenza virus 3, dengue virus, Ebola virus, Parvovirus B19 Coxsackie A-16 virus, HSV-1, hepatitis A virus, hepatitis B virus, hepatitis C virus, hepatitis D virus, hepatitis B virus, human immunodeficiency virus, Coxsackie B1-B5, Influenza viruses A, B or C, LaCross virus, Lassavirus, rubeola virus Coxsackie A or B virus, Echovirus, lymphocytic choriomeningitis virus, HSV-2, mumps virus, Respiratory Synytial Virus, Epstein-Barr Virus, Poliovirus Enterovirus, rabies virus, rubivirus, variola virus, WEE virus, Yellow fever virus and varicella zoster virus).

Alternatively, an immunoglobulin-containing fraction derived from a subject suffering from (or previously suffering from) a yeast or a fungal infection is useful for determining an immunogenic protein from said yeast or fungus. For example, a fungus or yeast that infects a host is selected from the group consisting of Aspergillus sp., Dermatophytes, Blastomyces dermatitidis, Candida sp., Histoplasma capsulatum, Sporothrix schenckii, Histoplasma capsulatum and Dematiaceous Fungi.

As used herein, the term “parasite” or “parasitological infection” shall be taken to mean an organism, whether unicellular or multicellular, other than a virus, bacterium, fungus or yeast that is capable of infecting another organism, for example a human. Examples of such parasites include, for example, a parasite selected from the group consisting of Ancylostoma ceylanicum, Ancylostoma duodenale, Ascaris lumbricoides, Balantidium coli, Blastocystis hominis, Clonorchis sinensis, Cyclospora cayetanensis, Dientamoeba fragilis, Diphyllobothrium latum, Dipylidium caninum, Encephalitozoon intestinalis, Entamoeba histolytica, Enterobius vermicularis, Fasciola hepatica, Enterobius vermicularis, Fasciola hepatica, Fasciolopsis buski, Giardia intestinalis (syn. Giardia lamblia), Heterophyes heterophyes, Hymenolepis diminuta, Hymenolepis nana, Isospora belli, Metagonimus yokogawai, Necator americanus, Opisthorchis felineus, Paragonimus westermani, Schistosoma haematobium, Schistosoma intercalatum, Schistosoma japonicum, Schistosoma mansoni, Taenia saginata, Trichuris trichiura, Babesia divergens, Plasmodium falciparum, Plasmodium malariae, Plasmodium ovale, Plasmodium vivax, Leishmania braziliensis and Leishmania donovani.

The present invention is also useful for, for example, identifying a protein against which a subject suffering from an autoimmune condition has raised an immune response. By “autoimmune condition” is meant that the immune system of a subject has raised one or more specific antibodies against one or more cellular components, eg. proteins, of the subject. The term autoimmune condition encompasses both autoimmune diseases and those aspects of a disease or disorder or infection that are associated with an autoimmune response. For example, in cases of chronic inflammation the immune system of a subject may raise specific antibodies against inflammatory proteins of that subject, as observed in a CF subject suffering from or that has previously suffered from an acute clinical exacerbation.

The present invention is useful for identifying a protein or fragment thereof against which a subject has raised an autoimmune response in an autoimmune disease. For example, the present invention is useful for identifying an immunogenic protein or fragment thereof in a subject suffering from an autoimmune disease selected of the group consisting of rheumatoid arthritis, multiple sclerosis, type-1 diabetes, inflammatory bowel disease, Crohn's Disease, ulcerative colitis, systemic lupus erythematosus, psoriasis, scleroderma, autoimmune thyroid disease, central nervous system vasculitis, and autoimmune myositis.

Furthermore, as stated supra the present invention is useful for identifying a protein against which a subject has raised an immune response during an autoimmune component of a disease. As exemplified herein, such an approach is useful for identifying an immunogenic protein from a subject against which the subject has raised an autoimmune response, wherein the subject suffers from an inflammatory condition, for example an acute clinical exacerbation of a CF subject

As used herein, the term “inflammatory condition” shall be understood to mean a state that is characterised by one or more changes in the physical appearance of functions of a portion of a subject, such as, for example, dilation of blood vessels with increased permeability and blood flow, exudation of fluids (e.g. plasma proteins), leukocytic infiltration, swelling and/or loss of function. Furthermore, an inflammatory condition is associated with the release of chemicals such as, for example, histamine, bradykinin, serotonin, inflammatory cytokines and others causing blood vessels to leak fluid into an inflamed tissue, resulting in localized swelling In one example, an inflammatory condition is a pulmonary inflammatory condition.

As used herein the term “acute clinical exacerbation”, “acute exacerbation”, “clinical exacerbation”, “exacerbation”, or “exacerbated state” in the context of a CF patient hall be understood to mean an exaggeration of a pulmonary symptom of CF.

In a further application of the present invention, the method is used to determine an immunogenic protein from a subject suffering from a cancer. Cancer cells aberrantly express some proteins or fragments of proteins. Accordingly, an antibody response is often raised against such aberrantly expressed proteins or fragments. Using the method of the present invention diagnostic and/or therapeutic markers of cancers are identified. For example, the present invention is useful for determining an immunogenic protein from a cancer selected from the group consisting of bladder cancer, breast cancer, colorectal cancer, endometrial cancer, head & neck cancer, leukemia, lung cancer, lymphoma, melanoma, non-small-cell lung cancer, ovarian cancer, prostate cancer, acute lymphocytic leukemia, adult acute myeloid leukemia, adult non-Hodgkin's lymphoma, brain tumor, cervical cancer, childhood sarcoma, chronic lymphocytic leukemia, chronic myeloid leukemia, oesophageal cancer, hairy cell leukemia, kidney cancer, liver cancer, multiple myeloma, neuroblastoma, oral cancer, pancreatic cancer, primary central nervous system lymphoma, skin cancer and small-cell lung cancer.

An example of the present invention additionally comprises immunizing a subject with one or more cells or an extract thereof comprising the immunogenic protein or fragment thereof to thereby elicity an immune response to the immunogenic protein or fragment thereof. This method is useful for, for example, determining an immunogenic protein from an infectious organism. For example, the subject is immunized with a sample of the infectious organism (eg. the organism itself whether alive or dead, or an extract derived from the organism eg. a protein extract). The subject is then allowed sufficient time to develop an immune response against said sample, and the method of the present invention performed to identify an immunogenic protein.

As will be apparent to the skilled artisan, the sample may be administered in the presence of a compound that enhances the level of an immune response against the sample, for example, an adjuvant. Adjuvants are known in the art and include, for example, Freund's complete or incomplete adjuvant, lysolecithin or dinitrophenol.

For example, a subject is immunized with one or more cells selected from the group consisting of viral cells, bacterial cells, yeast cells, fungal cells and cells from a parasite. Suitable cell extracts are, for example, selected from the group consisting of an extract from a virus, an extract from a bacterium, an extract from a yeast, and extract from a fungus and an extract from a parasite or mixtures thereof. Examples of such infectious organisms are described herein. For example, the present inventors have demonstrated that by immunizing a chicken with an extract from Mycobacterium (ie. Mycobacterium tuberculosis) specific antibodies are raised against proteins from that organism in an egg produced by the chicken.

In an alternative form of the invention the subject is immunized with, for example, a cancer cell.

Obtaining an Immunoglobulin-Containing Fraction

The method of the invention comprises obtaining the protein complex or immunoglobulin-containing fraction from a subject, and/or from a biological sample derived from or produced by the subject. For example, the protein complex or immunoglobulin-containing fraction is obtained by a process comprising separating or purifying a sample from or produced by the subject to thereby provide said protein complex or immunoglobulin-containing fraction.

Methods for separating or purifying a sample from or produced by the subject to thereby provide an immunoglobulin-containing fraction are known in the art and include, for example, precipitation using, for example, ethanol, polyethylene glycol, lyotropic (anti-chaotropic) salts such as ammonium sulfate and potassium phosphate.

Alternatively, or in addition, an immunoglobulin-containing fraction is isolated or purified from a biological sample by ion exchange chromatography or affinity chromatography, essentially as described in Burnouf and Radosevich, J Biochem Biophys Methods, 49(1-3), 575-86, 2001.

A method that facilitates isolation or purification of an immunoglobulin containing fraction comprises contacting the sample with one or more compounds capable of binding an immunoglobulin for a time and under conditions sufficient for binding to occur and isolating the compound.

While not essential to the performance of the method of the present invention, one or more compounds that is/are previously immobilized on a solid support, matrix or resin, such as, for example, a solid support, matrix or resin selected from the group consisting of cellulose bead, agarose, nylon, magnetic particle, paramagnetic particle and polymeric resin facilitates obtaining an immunoglobulin-containing fraction. Such immobilized compounds facilitate more rapid isolation of protein complex or immunoglobulin-containing fraction and allows for washing of the solid support to remove non-specifically bound or unbound components of a biological sample, eg. a protein.

For example, an immunoglobulin containing fraction is isolated or purified from a biological sample by hydrophobic chromatography, essentially as described in, for example, Doellgast and Plout, Immunochemistry, 13(2), 135-139, 1976. Such a method utilises a matrix that binds an immunoglobulin in the presence of lyotropic salts. Lyotropic salts are added to a biological sample derived from a subject and this sample contacted to said matrix. An immunoglobulin is then released from said matrix by reducing the concentration of lyotropic salts in a sample in a stepwise manner.

Thiophilic adsorption chromatography, essentially as described in Porath et al, FEBS Letters, 185, 306, 1985 and Knudsen et al, Analytical Biochemistry, 201, 170, 1992 is also useful for isolating an immunoglobulin-containing fraction from a biological sample. This method essentially comprises the use of divinyl sulfone activated agarose to which has been bound one or more ligand comprising a free mercapto-group. These ligands specifically bind an immunoglobulin in the presence of potassium sulfate. Such ligands include, for example 2-mercaptopyridine, 2-mercaptopyrimidine, and 2-mercaptothiazoline. Again, an immunoglobulin is released from a ligand by reducing the concentration of a lyotropic salt (ie potassium sulfate) in a sample.

Thiophilic resin is commercially available, for example, from BD Biosciences. In using such a thiophilic resin, an immunoglobulin containing sample is mixed with a salt, such as, for example, potassium sulfate, sodium sulfate and/or ammonium sulfate. Samples are then contacted with a thiophilic resin for a time and under condition sufficient for binding of an immunoglobulin in the sample. Samples are optionally washed to remove unbound or non-specifically bound protein and immunoglobulin isolated from the resin by elution with a low concentration of salt. Thiophilic resins permit purification of immunoglobulin by, for example, gravity-flow purification or batch-flow purification.

A matrix, such as, for example, that described in U.S. Pat. No. 6,498,016 is also useful for the isolation of an immunoglobulin-containing fraction from a biological sample. Such a matrix comprises a solid phase backbone, such as, for example cellulose, agarose, dextran based beads or organic polymers; optionally a spacer element; and a ligand comprising an aromatic or a heteroaromatic group, preferably, a benzene ring fused with a heteroaromatic ring system. Such a matrix does not require the use of a lyotropic salt, rather it is capable of binding an immunoglobulin under neutral conditions. An immunoglobulin is eluted or dissociated from such a matrix using conditions known in the art, such as, for example, washing the matrix with a buffer with a reduced pH, for example glycine, pH 3.

Recombinant or synthetic protein or peptide ligands are also useful for the isolation of an immunoglobulin-containing fraction. Such ligands are known in the art and described, for example, in Ngo and Khatter, Appl Biochem Biotechnol. 30:111-119, 1999; Verdoliva et al., J Immunol Methods. 271:77-88, 2002 or Kabir, Immunol Invest. 31:263-278, 2002.

Alternatively, or in addition, an immunoglobulin-containing fraction is separated from other constituents by affinity chromatography on Kaptive-M™-Sepharose. Those skilled in the art are aware that IgM binds to Kaptiv-M, the active constituent of which is a peptidomimetic compound that binds to IgM. Accordingly, a fraction comprising IgM and an immunogenic protein bound by IgM is isolated using this method.

Alternatively, MBP-Sepharose is used. Those skilled in the art are aware that MBP binds to mannose residues present on the IgM Fc5μ region, and, as a consequence, is specific for IgM. The initial step of binding is performed under native conditions so as not to perturb any protein-protein interaction (e.g., MBP bound to antibody, or alternatively or in addition, an antibody-antigen interaction). IgM and an antigenic protein are eluted from the affinity matrix using a dissociating buffer, such as, for example, a buffer comprising a high salt concentration (e.g., 3M MgCl₂ in HEPES pH 7.2) that releases the antibodies and immunogenic proteins as unbound components.

Alternatively, an immunoglobulin-containing fraction, for example, an immunoglobulin G fraction is isolated from a biological sample essentially as described in Stewart et al., Vox Sanguinis, 83: 332-338, 2002. Essentially, this method is a membrane-based preparative electrophoretic technique that isolates proteins based on their molecular weight and charge. Polyacrylamide membranes with varying pore sizes are used for size exclusion, whilst the pH of the electrophoresis buffer charges proteins depending upon their pI. Using this method immunoglobulin is isolated in a one-step or a two-step process. This method allows for the processing of large amounts of biological sample for the isolation of an immunoglobulin-containing fraction.

Chromatographic media that comprise one or more synthetic ligands capable of binding an immunoglobulin-containing fraction, such as, for example, Alpha™ mixed-mode chromatographic media (LigoChem, Inc., Fairfield, N.J.) or LigoSep® HTLC chromatographic media (LigoChem)is also useful for affinity purification of an immunoglobulin-containing fraction.

As will be apparent to the skilled artisan, dye ligands are also useful for purification of an immunoglobulin-containing fraction or an immunoglobulin containing protein complex. Such dye ligands and methods for using same are described, for example, in Clonis, In: Clonis et al Eds. Reactive Dyes in Protein and Enzyme Technology, London. MacMillan Press, 1987.

IgY is purified, for example, using a kit available from, for example, Afiland (Belgium) or Pierce (Rockford, Ill.). Such kits are preferably useful for isolating an IgY fraction from an egg or a derivative thereof or an extract thereof, eg. an egg yolk. Alternatively, a thiophilic resin is useful for isolating and IgY-containing fraction from an egg or a derivative thereof or an extract thereof.

As exemplified herein, protein G and/or protein A and/or protein L are useful for isolating an immunoglobulin containing fraction. Methods for isolating an immunoglobulin-containing fraction or a protein complex comprising immunoglobulin using protein G are known in the art and are described, for example, in Bjorck and Kronvall, J. Immunol. 33(2), 969-974, 1984. Methods for isolation of an immunoglobulin-containing fraction or a protein complex comprising immunoglobulin using protein A are known in the art and are described, for example, in Hjelm et al, FEBS Lett 28(1) 73-76 1972. Methods for isolating an immunoglobulin-containing fraction or a protein complex comprising immunoglobulin using protein L are known in the art and are described, for example, in Akerstrom and Bjorck J Biol Chem 264(33) 19740-19746, 1989.

An immunoglobulin-containing fraction is purified, for example, by affinity chromatography using, for example, a matrix, solid support, or resin bound to protein A and/or protein G and/or protein L, eg., protein-A Sepharose and/or protein-G Sepharose and/or protein L Sepharose (each of which are available from Amersham Pharmacia) or protein G agarose and/or protein A agarose and/or protein L agarose (each of which are available from Sigma Aldrich) or a magnetic bead conjugated to protein G and/or protein A (available from New England Biolabs). The initial step of binding is performed under native conditions so as not to perturb any protein-protein interaction (e.g., protein-A or protein-G bound to antibody, which is in turn bound to an immunogenic protein). The matrix is optionally be washed to remove any unbound or non-specifically bound protein. Antibodies are eluted from the protein-A, protein-G or protein-L using a dissociating buffer, such as, for example, a buffer comprising a high salt concentration (e.g., 3M MgCl₂ in HEPES pH 7.2) that releases the antibodies as unbound components.

As exemplified herein, an immunoglobulin-containing fraction is isolated using protein G or protein A.

As used herein, the term “protein G” shall be taken to include a protein comprising one or more natural IgG-binding domains of protein G, a hybrid or fusion protein comprising an IgG-binding domain of a native or naturally-occurring protein G, or a mutant or variant of a native or naturally-occurring protein G that retains the ability of native protein G to bind IgG, or a fragment of a native or naturally-occurring protein G that retains the ability of native protein G to bind IgG. By way of exemplification, the amino acid sequence of a Protein G from Streptococcus is set forth in SEQ ID NO: 1.

An exemplary form of Protein G is derived from Streptococcus sp. Lancefield Group G. This protein has a molecular weight of approximately 23-kDa. Preferably, Protein G binds the Fc portion of IgGs from a variety of species such as, for example, human, and mouse.

As used herein, the term “protein A” shall be taken to include a protein comprising one or more natural IgG-binding domains of protein A, a hybrid or fusion protein comprising an IgG-binding domain of a native or naturally-occurring protein A, or a mutant or variant of a native or naturally-occurring protein A that retains the ability of native protein A to bind IgG, or a fragment of a native or naturally-occurring protein A that retains the ability of native protein A to bind IgG. By way of exemplification the amino acid sequence of Staphylococcus aureus Protein A is set forth in SEQ ID NO: 2.

For example, a protein A comprises approximately five homologous IgG binding domains, each made up of approximately 60 amino acids. An exemplary form of Protein A has an isoelectric point of approximately 5.1.

In one example, Protein A is capable of binding to the Fc region of an immunoglobulin molecule.

Preferred Protein A is capable of binding rabbit, pig, mouse, rat, sheep, horse, goat, cat, dog, human IgG1 and/or IgG2 and/or IgG4 and/or IgM and/or IgA and/or IgE.

As used herein, the term “protein L” shall be taken to include a protein comprising one or more natural antibody light-chain-binding domains of protein L, a hybrid or fusion protein comprising an antibody light-chain-binding domain of a native or naturally-occurring protein L, or a mutant or variant of a native or naturally-occurring protein L that retains the ability of native protein L to bind an antibody light-chain, or a fragment of a native or naturally-occurring protein L that retains the ability of native protein L to bind an antibody light-chain. An exemplary Protein L is set forth in SEQ ID NO: 3.

An exemplary Protein L is derived from Peptostreptococcus magnus. Such a protein has a molecular weight of approximately 36-kDa and contains four immunoglobulin binding domains. Such an exemplary Protein L primarily binds to immunoglobulin kappa light chains.

Protein G and/or protein A and/or protein L referred to herein are obtained from a commercial source, or alternatively, produced by conventional means. Commercial sources will be known to those skilled in the art.

For example, protein G, protein A and/or protein L are available from Amersham-Pharmacia, Castle Hill, NSW, Australia.

Alternatively, protein G or protein A or protein L are isolated using the methods described in U.S. Pat. No. 4,945,157, U.S. Pat. No. 6,555,661 or U.S. Pat. No. 4,876,194 respectively. Alternatively, recombinant protein G and/or protein A and/or protein L are produced using techniques known in the art, as described, for example, in Sambrook et al (In: Molecular Cloning: A Laboratory Manual, Cold Spring Harbor Laboratories, New York, Second Edition (1989), whole of Vols I, II, and III). For example recombinant protein G may be produced using a method described in U.S. Pat. No. 5,082,773.

As will be apparent to the skilled artisan, attaching protein G, protein A and/or protein L to a solid support, resin or matrix facilitates affinity purification of an immunoglobulin-containing fraction or a protein complex comprising an immunoglobulin or mixtures thereof. A solid support, a resin or a matrix suitable for attachment of protein G, protein A and/or protein L include, for example, a solid phase support selected from the group consisting of a polymer having one or more hydroxyl groups, either free or in esterified form, such as agarose, cellulose, including cellulose esters (such as cellulose nitrate, diazocellulose, cellulose acetate and cellulose propionate), or acrylamide polymers or copolymers (such as polyacrylamide or acrylamide), microtitre plates, glass, polystyrene, polypropylene, polyethylene, dextran, nylon, agar, starch, or a chemically active membrane having a large surface area comprising a hydrophobic, microporous, skinless, polyamide membrane which is chemically bound to a residue of an activating agent which is capable of immobilizing a protein G, protein A and/or protein L.

Protein G. protein A and/or protein L is immobilized on a solid support, resin or matrix according to methods known to those of ordinary skill in the art. For example, a protein G, protein A and/or protein L is coated or bonded, either covalently or by adsorption, to a solid phase. Methods for immobilizing a protein to a solid phase support are taught, for example, in U.S. Pat. No. 3,652,761, U.S. Pat. No. 3,879,262, U.S. Pat. No. 3,986,217, and U.S. Pat. No. 4,693,985. For example, a protein G, protein A and/or protein L are immobilized to tresyl activated or cyanogen bromide activated agarose or a maleimide-activated agarose support. This support is prepared, for example, by treating agarose modified to contain primary amino groups (in particular, AH-Sepharose, Pharmacia Co.) with sulfosuccinimidyl-4-(p-maleimidophenyl) butyrate.

Immobilised protein G and/or protein A and/or protein L is useful for affinity purification of an immunoglobulin. Affinity purification techniques are known in the art and are described, for example, in Scopes (In: Protein purification: principles and practice, Third Edition, Springer Verlag, 1994). Methods for affinity purification typically involve contacting a biological sample isolated from a subject or a sample derived from or produced by the subject to an immobilised Protein G, Protein A and/or Protein L, and, (optionally) following washing to remove any unbound or non-specifically bound protein, eluting an immunoglobulin that is bound to a Protein G and/or Protein A and/or Protein L.

Alternatively, a protein G and/or protein A and/or protein L is covalently bound to a molecule, such as, for example, biotin. Accordingly, an affinity purification method involving such a conjugated protein G, protein A and/or protein L, uses, for example, streptavidin that has been conjugated to a solid support to bind, or capture a conjugated protein G and/or protein A and/or protein L.

Alternatively, a protein G and/or protein A and/or protein L is covalently linked to a magnetic or paramagnetic bead, such as for example a Dynabead® (available from Dynal Biotech, Oslo, Norway). By contacting a biological sample derived from or produced by a subject with such a linked protein G, protein A and/or protein L and subsequently exposing the sample to a magnetic or paramagnetic field an immunoglobulin fraction or a protein complex comprising an immunoglobulin or mixtures thereof is isolated.

Alternatively, protein G, protein A and/or protein L is contacted with a biological sample derived from a subject for a time and under conditions that allow said protein G, protein A or protein L to bind to an immunoglobulin-containing fraction. This sample is then contacted with an antibody that specifically binds to a protein G and/or protein A and/or protein L. Preferably, this antibody is bound to a solid support or another means that facilitates isolation of a protein G and/or protein A and/or protein L from a biological sample, such as for example, agarose, a plastic solid support or a glass solid support. Polyclonal antibodies that specifically bind protein G or protein A or protein L are available from, for example, Sapphire Bioscience, Crows Nest, NSW, Australia.

A mimetic of protein G or protein A or protein L is also useful for isolating a protein complex comprising an immunoglobulin or mixtures thereof or an immunoglobulin containing fraction thereof. Such a mimetic is known in the art and/or described, for example, in Kabir, Immunol Invest.; 31:263-78, 2002 or Dowd et al., Nat. Biotechnol. 16:190-5, 1998. For example, a peptide mimetic of protein A comprises an amino acid sequence EQQNAFYEILHLPNLNEEQR (SEQ ID NO: 4) or RTYRTYRTYRTYKKKG (SEQ ID NO: 5)

Alternatively, an immunogenic protein is obtained from a biological sample using a protein chip. To produce such a protein chip, a protein that is able to bind an immunoglobulin of interest, such as, for example protein G, protein A and/or protein L is bound to a solid support such as for example glass, polycarbonate, polytetrafluoroethylene, polystyrene, silicon oxide, metal or silicon nitride. This immobilization is either direct (e.g. by covalent linkage, such as, for example, Schiff's base formation, disulfide linkage, or amide or urea bond formation) or indirect. Methods of generating a protein chip are known in the art and are described in for example U.S. Patent Application No. 20020136821, 20020192654, 20020102617 and U.S. Pat. No. 6,391,625. To bind a protein to a solid support it is often necessary to treat the solid support so as to create chemically reactive groups on the surface, such as, for example, with an aldehyde-containing silane reagent. Alternatively, a protein that is able to bind an immunoglobulin of interest may be captured on a microfabricated polyacrylamide gel pad and accelerated into the gel using microelectrophoresis as described in, Arenkov et al. Anal. Biochem. 278:123-131, 2000.

Linking an Immunoglobulin-Containing Fraction to an Immunoglobulin Binding Compound

In one form, the method of the invention additionally comprises linking of the immunoglobulin-containing fraction or protein complex comprising an immunoglobulin to the compound used to bind the immunoglobulin.

A means by which to bind the immunoglobulin-containing fraction to a compound is by using hydrazide. Such a method is performed essentially as described in O'Shannessy and Hoffmann, Biotechnol. Appl. Biochem. 9(6), 488-496, 1987. Essentially this method comprises isolating an immunoglobulin using a method known in the art and/or described herein, and oxidising said immunoglobulin with sodium periodate. This oxidisation causes formation of aldehydes on any oligosaccharide moiety. An oxidised sample is then contacted with a hydrazide-derivatized solid support. This causes the formation of a stable hydrazone linkage between an oxidised immunoglobulin and said solid support. A suitable solid supports includes, for example, agarose, a glass bead or a polystyrene, polypropylene or polycarbonate bead or a microtitre plate. As a hydrazone bond is stable, even at low pH ranges, such a method allows for dissociation of an immunogenic protein from the immunoglobulin-containing fraction by a method known in the art and/or described herein.

In one exemplified form of the method of the invention linking an immunoglobulin-containing fraction or a protein complex to a compound that binds an immunoglobulin comprises contacting a cross-linking agent with the one or more compounds having the immunoglobulin bound thereto for a time and under conditions sufficient for covalent linkage to occur between a compound and the immunoglobulin to occur.

For example, a protein comprising an immunoglobulin or mixtures thereof or an immunoglobulin containing fraction thereof is linked to an immunoglobulin binding compound using a photoreactive cross-linking reagent. Such photoreactive cross-linking reagents include, for example, a aryl azide (upon illumination an aryl azide generates reactive intermediates that form bonds with nucleophilic groups), a fluorinated aryl azides (that upon UV photolysis generates reactive nitrenes) or a benzophenone derivative. For example, a suitable photoreactive cross-linking reagent is selected from the group consisting of N-((2-pyridyldithio)ethyl)-4-azidosalicylamide (PEAS), 4-Azido-2,3,5,6-tetrafluorobenzyl amine, a reactive derivative of 4-azido-2,3,5,6-tetrafluorobenzoic acid, Benzophenone maleimide and benzophenone isothiocyanate. Following addition of a photoreactive cross-linking reagent the sample is exposed to UV light for a time and under conditions sufficient for the formation of a bond between the immunoglobulin binding compound and the immunoglobulin fraction bound thereto.

Alternatively, a suitable cross-linking agent is, for example, selected from the group consisting of an imidoester cross-linker, a N-hydroxysuccinimide cross-linker, a maleimide cross-linker, a haloacetyl cross-linker, a hydrazide cross-linker, and a carbodiimide cross-linker.

For example, an imidoester cross-linker react with amine groups at an alkaline pH causing formation of an amidine bond. A homobifunctional imidoester is useful for cross-linking proteins as the net electric charge of the protein is maintained after cross-linking. A suitable imidoester cross-linker is selected from the group consisting of dimethyladipimidate-2.HCl (DMA), dimethylpimelimidate.HCl (DMP), dimethylsuberimidate.2HCl (DMS) and dimethyl 3,3′-dithiobispropionimidate-2.HCl (DTBP).

Maleimides are also useful for cross-linking an immunoglobulin-containing fraction or a protein complex comprising an immunoglobulin with an immunoglobulin binding compound. Maleimides specifically react with a sulfhydryl group at an approximately neutral pH. However, maleimides also react with amine groups, albeit at a slower rate than with sulfhydryl groups. Maleimides form a stable thioester linkage with the reacted sulfhydryl group that cannot be cleaved under normal physiological conditions. An example of a suitable maleimide cross-linker is selected from the group consisting of succinimidyl 4-(N-maleimidomethyl)cyclohexane-1-carboxylate (SMCC), sulfo-succinimidyl 4-(N-maleimidomethyl)cyclohexane-1-carboxylate (sulfo-SMCC), m-maleimidobenzoyl-N-hydroxysuccinimide ester (MBS), sulfo-maleimidobenzoyl-N-hydroxysuccinimide ester (sulfo-MBS), succinimidyl 4-(p-maleimidophenyl)-butyrate (SMBP), sulfo-succinimidyl 4-(p-maleimidophenyl)-butyrate (sulfo-SMBP), bismaleimidohexane (BMH), N-(g-maleimidobutyryloxy)succinimide ester (GMBS) and sulfo-N-(g-maleimidobutyryloxy)succinimide ester (sulfo-GMBS).

Alternatively, or in addition, a haloacetyl cross-linker is used to cross-link an immunoglobulin-containing fraction or a protein complex comprising an immunoglobulin with an immunoglobulin binding compound. The majority of commonly used α-haloacetyl cross-linker comprise an iodoacetyl group. Reaction of such a iodoacetyl group with a sulfhydryl group at a physiological pH proceeds by nucleophilic substitution of iodine with a thiol producing a stable thioester linkage. A haloacetyl cross-linker suitable for the method of the present invention includes, for example, a N-succinimidyl (4-iodacetyl)aminobenzoate (SLAB) or sulfo-N-succinimidyl (4-iodacetyl)aminobenzoate (sulfo-SIAB).

A pyridyl disulfide cross-linker useful for crosslinking an immunoglobulin-containing fraction or a protein complex comprising an immunoglobulin with an immunoglobulin binding compound is, for example, selected from the group consisting of 1,4-Di-[3′-2′-pyridyldithio-(propionamido)butane] (DPDPB), 4-succinimidyl-oxycarbonyl-a-(2-pyridyldithio)toluene (SMPT), sulfosuccinimidyl-6-[a-methyl-a(2-pyridyldithio)tluamido]hexanoate (sulfo-LC-SMPT), N-succinimidyl-(pyrodyldithio)-propionate, succinimidyl 6-[3-(2-pyridyldithio)-propionamido]hexanoate (LC-SPDP), sulfosuccinimidyl 6-[3-(2-pyridyldithio)-propionamido]hexanoate (sulfo-LC-SPDP) and 3-(2-pyridyldithio)-propionyl hydrazide (PDPH). Pyridyl disulfides react with alipathic thiols at a weakly acidic pH (eg. between about pH 4 to pH 5), however neutral pH can be used, to produce a disulfide bond.

A carbodiimide cross-linker is also useful for linking an immunoglobulin-containing fraction or a protein complex comprising an immunoglobulin with an immunoglobulin binding compound. Carbodiimides couple carboxyls to primary amines or hydrazides, causing the formation of amide or hydrazone bonds. Carbodiimides do not form a cross-bridge between coupled molecules, unlike many other cross-linking reagents. An example of a suitable carbodiimide cross-linker is selected from the group consisting of 1-ethyl-3-(3-dimethylaminopropyl)-carbodiimide hydrochloride (EDC), N,N′-dicyclohexylcarbodiimide (DCC) and 4-(p-azidosalicylamido)-butylamine.

As exemplified herein a N-hydroxysuccinimide (NHS) cross-linker is useful for linking an immunoglobulin-containing fraction or a protein complex comprising an immunoglobulin with an immunoglobulin binding compound. A NHS cross-linker generally interacts with primary amines, such as, for example an α-amine group on the N-terminus of a protein, however ε-amines also interact with NHS-esters. A covalent amide bond is formed when the NHS cross-linker interacts or reacts with a primary amine and releases N-hydroxysuccinimide. By way of exemplification, a suitable NHS cross-linker is selected from the group consisting of disuccinimidyl glutarate (DSG), disuccinimidyl suberate (DSS), Bis(sulfosuccinimidyl) suberate BS₃), dithiobis(succinimidyl propionate) (DSP), 3,3′-dithiobis(succinimidyl propionate) (DTSSP), ethylene glycobis(succinimidyl succinate) (EGS), ethylene glycobis(sulfo-succinimidylsuccinate) (sulfo-EGS), disuccinimidyl tartarate (DST), disulfosuccinimidyl tartarate (sulfo-DST), bis[2-(succinimidyloxy-carbonyloxy) ethyl]sulfone (BSOCOES), bis[2-(sulfosuccinimidyloxy-carbonyloxy) ethyl]sulfone (sulfo-BSOCOES), succinimidyl 4-(N-maleimidomethyl) cyclohexane-1-carboxylate (SMCC), sulfo-succinimidyl 4-(N-maleimidomethyl) cyclohexane-1-carboxylate (sulfo-SMCC), m-maleimido benzoyl-N-hydroxysuccinimide ester (MBS), m-maleimido benzoyl-N-hydroxysulfosuccinimide ester (sulfo-MBS), succinimidyl 4-(p-maleimidophenyl)-butyrate (SMBP), sulfo-succinimidyl 4-(p-maleimidophenyl)-butyrate (sulfo-SMBP), bismaleimidohexane (BMH), N-(g-maleimidobutyryloxy)succinimide ester (GMBS) and N-(g-maleimidobutyryloxy) sulfosuccinimide ester (sulfo-GMBS).

As exemplified herein, disuccinimidyl suberate (DSS) is useful for cross-linking an immunoglobulin containing protein complex or an immunoglobulin-containing fraction with an immunoglobulin binding compound, such as, for example protein G. For example, an immunoglobulin-containing fraction is isolated from a biological sample derived from a subject (eg. serum) using, for example, protein G Sepharose. The bound immunoglobulin-containing fraction is then cross-linked to the protein G by contacting the protein G bound immunoglobulin-containing fraction with a suitable amount of DSS for a time and under conditions for an amide bond to form between the protein G and the immunoglobulin-containing fraction.

Separating an Immunogenic Protein from an Immunoglobulin

As the immunogenic protein or fragment thereof is to be identified, in one form of the invention it is desirable to separate said immunogenic protein or fragment thereof from the immunoglobulin-containing fraction to which it is bound by virtue of an antigen-antibody interaction. For example, the immunogenic protein or fragment thereof is separated from an immunoglobulin-containing fraction or a protein complex comprising an immunoglobulin or mixtures thereof by contacting the protein complex comprising an immunoglobulin or mixtures thereof or an immunoglobulin-containing fraction thereof with a compound that disrupts the antigen-antibody interaction for a time an under conditions sufficient to disrupt the antigen-antibody interaction.

For example, an immunogenic protein is dissociated from an immunoglobulin to which it is bound, prior to further analysis. An immunogenic protein is considered to be dissociated from an immunoglobulin, when it is no longer bound by said immunoglobulin, that is an immunoglobulin does not form a non-covalent bond (as described supra) with the immunogenic protein or fragment thereof. Methods for separating a protein from an immunoglobulin (eg. an antibody) are known in the art and are described, for example, in Scopes (In: Protein purification: principles and practice, Third Edition, Springer Verlag, 1994). For example, an immunogenic protein is separated from an immunoglobulin-containing fraction or a protein complex comprising an immunoglobulin or mixtures thereof by altering or modifying the pH of a sample that comprises said immunoglobulin-containing fraction or a protein complex comprising an immunoglobulin or mixtures thereof. The pH of said sample is altered using, for example, glycine (eg., with a pH of approximately 3) or triethanolamine (with a pH of approximately 11))

Alternatively, or in addition, an immunogenic protein is separated from an immunoglobulin-containing fraction or a protein complex comprising an immunoglobulin or mixtures thereof by increasing the salt concentration of a sample comprising the immunoglobulin-containing fraction or a protein complex comprising an immunoglobulin or mixtures thereof (for example, with 5M Lithium Chloride).

Treating a sample comprising an immunoglobulin-containing fraction or a protein complex comprising an immunoglobulin or mixtures thereof with an ionic detergent (for example sodium-dodecyl sulfate (SDS)), and/or with a dissociating agent (for example urea) and/or with a chaotropic agent (for example thiocyanate) also separates an immunogenic protein or fragment thereof from an immunoglobulin-containing fraction or a protein complex comprising an immunoglobulin or mixtures thereof.

As will be apparent to a skilled artisan a combination of such methods is also useful for separating an immunogenic protein or fragment thereof from an immunoglobulin-containing fraction or a protein complex comprising an immunoglobulin or mixtures thereof.

In an exemplified form of the invention, an immunogenic protein is dissociated from an immunoglobulin-containing fraction or a protein complex comprising an immunoglobulin or mixtures thereof by reducing the pH of a sample comprising said immunogenic protein bound to said immunoglobulin-containing fraction or a protein complex comprising an immunoglobulin or mixtures thereof with glycine. For example, the glycine is at a pH of about 1.5 to a pH of about 4, more preferably, a pH of about 1.9 to a pH of about 2.7 and most preferably a pH of about 2.3 to a pH of about 2.7.

Alternatively, an immunogenic protein is isolated or dissociated from an immunoglobulin-containing fraction using caprylic acid and ammonium sulphate precipitation. Using such agents provides preparations that comprise essentially an immunoglobulin or an immunogenic protein.

By dissolving the immunoglobulin-containing fraction in a dissociating buffer such as, for example, a high-salt buffer (e.g., 3M MgCl₂ in/HEPES pH 7.2), an immunogenic protein or fragment thereof is released as unbound components. The immunogenic protein fraction and the immunoglobulin-containing fraction are then separated by, for example size exclusion chromatography, for example, using the dissociating buffer as an eluant to maintain the immunoglobulin components as unbound components.

Alternatively, an immunoglobulin-containing fraction or a protein complex comprising an immunoglobulin or mixtures thereof is subjected to free-flow electrophoresis under denaturing conditions. A biological sample is clarified and the proteins are precipitated under conditions that leave immunoglobulin in solution. The immunoglobulin-containing fraction is then precipitated and redissolved in a suitable buffer, applied to a free-flow electrophoresis (FFE) device (e.g., Octopus™, Tecan™) for separation by continuous solution-phase isoelectric focusing, for example, essentially as described by Hoffman et al., Proteomics 1, 807-818, 2001). Fractions are obtained, eg., corresponding to an immunogenic protein or a fragment thereof, and exchanged into a suitable buffer (e.g., PBS) using PD-10 or fast-desalting columns (Amersham Biosciences) prior to further analysis to determine the identity of the immunogenic protein using methods well known in the art and/or described herein.

Isolation of an Immunogenic Protein and/or an Immunoglobulin-Containing Fraction

Alternatively, or in addition to separating an immunogenic protein from an immunoglobulin-containing fraction or a protein complex comprising an immunoglobulin or mixtures thereof, such an immunogenic protein is, for example, isolated from the immunoglobulin-containing fraction or a protein complex comprising an immunoglobulin or mixtures thereof to which it was bound by virtue of an antigen-antibody interaction.

Accordingly, the isolation of the immunogenic protein or fragment thereof and/or the immunoglobulin-containing fraction or a protein complex comprising an immunoglobulin or mixtures thereof is performed either after first separating said proteins/fractions or without separating said proteins/fractions.

A method for separating proteins is known in the art and described, for example, in Scopes (In: Protein purification: principles and practice, Third Edition, Springer Verlag, 1994).

For example, an immunogenic protein is separated from an immunoglobulin following dissociation using native gel electrophoresis. As used herein the term “native gel electrophoresis” shall be taken to mean any form of electrophoresis that is performed under conditions that do not denature a protein, that is a protein that is electrophoresed retains its native size, conformation and/or charge. Accordingly, mobility of a protein using native gel electrophoresis depends upon both the charge of the protein and the hydrodynamic size of the protein. Such a method is of use in the separation of an immunogenic protein and an immunoglobulin, as, not only does native gel electrophoresis maintain the size, shape and charge of a protein, this method also allows proteins that normally interact to remain bound. As an immunoglobulin comprises two heavy chains and two light chains, it is expected that an immunoglobulin has a molecular weight of at least about 150 kDa (corresponding to the predicted molecular weight of IgG). Accordingly, electrophoresis of a sample prepared using the previously described method facilitates separation of an immunogenic protein or fragment thereof from an immunoglobulin-containing fraction.

For instance, a sample comprising an immunogenic protein and an immunoglobulin are electrophoresed using one dimensional native gel electrophoresis using techniques known in the art. In such cases proteins are merely separated by their molecular weight and charge. Accordingly, such a method is of use in separating an immunoglobulin from a smaller immunogenic protein or fragment thereof. The immunogenic protein is then identified using a method known in the art and/or described herein.

Alternatively, a sample comprising an immunogenic protein and an immunoglobulin is electrophoresed using native two-dimensional gel electrophoresis. For example proteins are separated in one dimension using isoelectric focussing. Using such a method, proteins are separated by their isoelectric point, that is the pH at which the net charge of a protein is equal to zero. To separate proteins by their isoelectric point a sample is electrophoresed in a gel that comprises a pH gradient. Under such conditions, a protein will move to a position on said gradient where its net charge is equal to zero. Following isoelectric focussing proteins are separated according to their mass, using standard native gel electrophoresis. Accordingly, such a method is of use in the separation of an immunoglobulin from an immunogenic protein.

Alternatively, an immunogenic protein is isolated from an immunoglobulin by dissociating said immunogenic protein from an immunoglobulin using a method known in the art and/or described herein and separating said immunogenic protein or a fragment thereof and said immunoglobulin using a gel filtration column. Such columns are available from commercial sources, such as, for example, Sigma-Aldrich or Amersham-Pharmacia. Methods of gel filtration are known in the art and are described, for example, in Scopes (In: Protein purification: principles and practice, Third Edition, Springer Verlag, 1994). Gel filtration chromatography separates proteins based upon their size. Such a method comprises contacting a sample to a column that comprises a solid matrix that consists of a specified pore size. Proteins that are of a sufficiently low molecular weight move through these pores and are said to be included, while those that do not are excluded. Proteins are eluted from said column, with those that are excluded eluting prior to those that are included. Accordingly, as an immunoglobulin in its native state is a relatively large molecule, it will elute before an immunogenic protein or fragment thereof that has a lower molecular weight. Following collection of a sample comprising a immunogenic protein or fragment thereof, said sample is analysed/identified using a method known in the art, and/or described herein. Alternatively, a sample comprising an immunogenic protein is separated using electrophoresis, for example native or denaturing one- or two-dimensional gel electrophoresis, prior to any analysis of said immunogenic protein.

Alternatively, an immunogenic protein is isolated from an immunoglobulin following dissociation from said immunoglobulin using other methods of size exclusion, such as for example, centrifugation using a size exclusion filter (for example as available from Millipore), high performance liquid chromatography or reverse phase chromatography, amongst others.

An immunogenic protein is also or alternatively separated from an immunoglobulin-containing fraction or a protein complex comprising an immunoglobulin or mixtures thereof following dissociation from said immunoglobulin using, for example, density gradient fractionation. Methods of fractionation using a density gradient are known in the art. For example, proteins are separated using ultracentrifugation, where a sample is added to a linear sucrose gradient ranging, for example, from 5% to 20% and subsequent centrifugation. Accordingly, proteins are separated with regard to centrifugal force, frictional force and buoyant force. Using such a method, an immunoglobulin is separated from an immunogenic protein or fragment thereof as it is a relatively large protein. Following separation from an immunoglobulin, an immunogenic protein is analysed using a method known in the art and/or described herein.

As exemplified herein, an immunogenic protein or fragment thereof is isolated from an immunoglobulin-containing fraction or a protein complex comprising an immunoglobulin or mixtures thereof using denaturing electrophoresis. Denaturing electrophoresis is performed as described supra, however, rather than being performed under native conditions, reagents that denature proteins are included in either or both the electrophoresis gel and in sample preparation. Accordingly, protein samples are denatured using, for example, detergent (eg SDS), or other denaturants (eg 2-mercaptoethanol, DTT and/or heat).

For example, an immunogenic protein is isolated from an immunoglobulin using reducing one-dimensional gel electrophoresis, using methods known in the art, and described, for example, in Scopes (In: Protein purification: principles and practice, Third Edition, Springer Verlag, 1994). In accordance with this embodiment, proteins are separated by their molecular weight. Accordingly, an immunogenic protein or fragment thereof that has a molecular weight different to both a heavy and a light chain of an immunoglobulin is readily detectable using this method.

In another embodiment, an immunogenic protein is isolated from an immunoglobulin using reducing two-dimensional gel electrophoresis. In accordance with this embodiment, proteins are separated, for example, by their isoelectric point or net charge and molecular weight. As such, this method is of use in determining an immunogenic protein or fragment thereof that has a different molecular weight and/or isoelectric point from that of an immunoglobulin light or heavy chain.

In accordance with either of the two previous embodiments, following separation of an immunogenic protein from an immunoglobulin using reducing electrophoresis, an immunogenic protein is identified using a method known in the art and/or described herein.

In accordance with any of the previously mentioned embodiments relating to dissociation or separation of an immunogenic protein from an immunoglobulin, a sample comprising an isolated immunogenic protein or fragment thereof may optionally be concentrated prior to further analysis. Methods of concentrating a protein are known to those skilled in the art, and include, for example, precipitation, freeze drying, use of funnel tube gels (TerBush and Novick, Journal of Biomolecular Techniques, 10(3); 1999), ultrafiltration or dialysis.

Identification of an Immunogenic Protein without Dissociation from an Immunoglobulin

The present invention provides for the identification of an immunogenic protein or fragment thereof that has not been isolated from an immunoglobulin-containing fraction or a protein complex comprising an immunoglobulin or mixtures thereof.

For example, an immunoglobulin-containing fraction is isolated from a biological sample derived from a subject using a method known in the art and/or described herein, and the Fc region of said immunoglobulin is cleaved using a protease. For example, a protease selected from the group consisting of, papain, elastase, SpeB and EndoS from Streptococcus pyogenes and pepsin. Both papain and elastase are commercially available from, for example, Merck, while pepsin is commercially available from Calzyme Laboratories, San Luis Obispo, Calif., USA.

In accordance with this embodiment, an immunogenic protein remains bound to a fragment of an immunoglobulin, and said complex is separated from the Fc region of said immunoglobulin. This sample is then analysed using a method known in the art and/or described herein

Alternatively, an immunogenic protein and a cleaved immunoglobulin are first separated using methods known in the art and/or described herein, such as, for example, non-reducing or reducing one- or two-dimensional gel electrophoresis, prior to analysis to determine the identity of the immunogenic protein.

Identification of an Immunogenic Protein or a Fragment Thereof

Following capture and/or separation and/or isolation of an immunogenic protein or fragment thereof whether with an immunoglobulin-containing fraction, or cleavage of an immunoglobulin and recovery of an immunogenic protein or fragment thereof, said immunogenic protein is analysed to determine the identity of said protein. Methods of analysis of a protein in determine the identity of a protein are known in the art and include, for example, a method selected from the group consisting of Edman sequencing, mixed peptide sequencing, mass spectrometry including MALDI-TOF, ESI and ion trap analysis amongst others.

For example, the identity of an immunogenic protein is identified using Edman sequencing (essentially as described by Edman, Arch. Biochem. Biophys., 22, 475-483, 1949) to determine the N-terminal sequence of an immunogenic protein and comparing this sequence to a known sequence Such a method is useful for determining the identity of an immunogenic protein or a fragment thereof. Preferably, an immunogenic protein or a fragment thereof is separated from a contaminating molecule, such as, for example another protein, prior to Edman sequencing. Following isolation of an immunogenic protein, the amino terminus of said protein is derivatized with phenylisothiocyanate under basic conditions. For example, the base used in this step is a non-nucleophile such as, for example, a triethylamine or diisopropylethylamine. This coupling step produces a phenylthiocarbamyl peptide or protein. The thiocarbonyl function of the phenylthiocarbamyl peptide or protein is a moderately strong nucleophile, and under acidic conditions it will cleave the carbonyl carbon of the adjacent peptide bond. This cleavage step results in the production of an anilothiazolinone of the terminal amino acid and leaves the original peptide or protein shortened by one amino acid residue. The anilothiazolinone of the terminal amino acid has different solubility properties from the peptide or protein. As such, it can be extracted and subjected to further analysis. The shortened peptide or protein again has a bare amino terminus, and, as a consequence, can be subjected to additional cycles of coupling, cleavage, and extraction.

The extracted anilothiazolinone of the terminal amino acid, however, is not stable. Under acidic aqueous conditions, anilothiazolinones rearrange rapidly to form more stable phenylthiohydantoins, which are amenable to analysis. A stable phenylthiohydantoin is then analyzed by, for example, UV absorption detection reverse phase high performance liquid chromatography, to determine the identity of the terminal amino acid.

Following determining the N-terminal sequence of an immunogenic protein, this sequence is compared to a database of sequences in order to determine whether or not the derived sequence is identical to or substantially identical to a known sequence. Such a database is available, for example, at NCBI.

As used herein the term “NCBI” shall be taken to mean the database of the National Center for Biotechnology Information at the National Library of Medicine at the National Institutes of Health of the Government of the United States of America, Bethesda, Md., 20894.

In determining whether or not two amino acid sequences fall within the defined percentage identity limits supra, those skilled in the art will be aware that it is possible to conduct a side-by-side comparison of the amino acid sequences. In such comparisons or alignments, differences will arise in the positioning of non-identical residues depending upon the algorithm used to perform the alignment. In the present context, references to percentage identities and similarities between two or more amino acid sequences shall be taken to refer to the number of identical and similar residues respectively, between said sequences as determined using any standard algorithm known to those skilled in the art. In particular, amino acid identities and similarities are calculated using software of the Computer Genetics Group, Inc., University Research Park, Maddison, Wis., United States of America, eg., using the GAP program of Devereaux et al., Nucl. Acids Res. 12, 387-395, 1984, which utilizes the algorithm of Needleman and Wunsch, J. Mol. Biol. 48, 443-453, 1970. Alternatively, the CLUSTAL W algorithm of Thompson et al., Nucl. Acids Res. 22, 4673-4680, 1994, is used to obtain an alignment of multiple sequences, wherein it is necessary or desirable to maximise the number of identical/similar residues and to minimise the number and/or length of sequence gaps in the alignment. Amino acid sequence alignments can also be performed using a variety of other commercially available sequence analysis programs, such as, for example, the BLAST program available at NCBI.

Alternatively, an immunogenic protein or fragment thereof is identified using mixed-peptide sequencing, as described in Damer et al, J. Biol. Chem. 273, 24396-24405, 1998. In accordance with this embodiment, an immunogenic protein is cleaved into peptides using cyanogen bromide or skatole and these peptides are sequenced using the Edman sequencing method.

As exemplified herein, an immunogenic protein or fragment thereof is identified using mass spectrometry. For example, an immunogenic protein is separated using electrophoresis and, optionally, the immunogenic protein is digested with a protease prior to analysis with mass spectrometry.

For example, following separation of an immunogenic protein using electrophoresis, said protein is digested in the gel in which electrophoresis occurred. In-gel digestion of a protein, peptide or polypeptide enables more of said protein to be recovered from a gel than other methods such as for example electroblotting. Accordingly, the increased quantity of an immunogenic protein facilitates analysis of said protein. Methods of in-gel digestion are known in the art and are described, for example, in Schevenko et al, Anal. Chem., 68, 850-858, 1997. Furthermore, kits that facilitate in-gel digestion of a protein are commercially available, for example, from Millipore, Billerica, Mass. 01821, USA.

Alternatively, or in addition, an immunogenic protein or a peptide thereof is purified and optionally concentrated prior to further analysis. For example, an immunogenic protein or a peptide thereof is purified using reverse-phase chromatography.

In an alternative example, an immunogenic protein is not electrophoresed, rather a sample dissociated from an immunoglobulin is used for analysis. Optionally, an immunogenic protein is digested with a protease, such as, for example, trypsin, to facilitate analysis of peptides of an immunogenic protein or fragment thereof. Accordingly, following purification, and optionally concentration, such a sample is analysed by mass spectrometry.

Following purification of an immunogenic protein or a peptide thereof, samples are ionised.

For example, a sample is ionised using electrospray ionisation (ESI), essentially as described in, for example Fenn et al, Science, 246, 64-71, 1989 or Wilm et al, Nature, 379, 466-469, 1996. The process of ESI forces a sample comprising an immunogenic protein or a fragment thereof into a mass spectrometer through a microcapillary tube. A potential difference between the chamber of the mass spectrometer and the microcapillary tube cause the sample comprising an immunogenic protein or a fragment thereof to be ejected from said tube as a fine mist. As the liquid in this mist evaporates (ie the solution in which a protein is suspended) the protein or peptide thereof becomes desolvated. Accordingly, a protein or peptide is converted to ions.

Alternatively, a sample is ionised using matrix assisted laser desorption/ionisation (MALDI), for example, essentially as described by, for example, Karas and Hillenkamp, Anal. Chem., 60, 2299-2301, 1988. For example, a sample comprising an immunogenic protein or fragment thereof is incorporated into a matrix, such as for example a-cyano-4-hydroxycinnamic acid, 3,5 dimethoxy-4-hydroxycinnamic acid (Sinapinic acid) or 2,5 dihydroxybenzoic acid (Gentisic acid). The sample and matrix are then spotted onto a metal plate and subjected to irradiation by a laser, promoting the formation of molecular ions. As will be apparent to those skilled in the art, variations of this method are clearly encompassed in the instant invention, such as, for example, atmospheric pressure MALDI.

As will be apparent to the skilled artisan other forms of ionization are clearly encompassed in the instant invention, for example, atmospheric pressure chemical ionization.

Following ionisation of a protein or peptide thereof the mass of these molecular ions are analysed.

For example, the mass of a molecular ion is analysed using a quadrupole mass analyser, or a linear quadrupole, essentially as described in Burlingame et al, Anal. Chem. 70, 674R-716R and references cited therein. This method transmits an ion through an electric field generated by an array of four metallic rods, to which rf and dc voltages are supplied. This voltage causes an ion to oscillate with the frequency of this oscillation depending upon the m/z value of the ion. Only those ions that show a stable oscillation, that is those that have a given m/z value as determined by the rod assembly, oscillation frequency, rf voltage and dc voltage, are retained for further analysis. Accordingly this facilitates analysis of the mass to charge ratio (m/z) of a peptide or protein. This is then compared to a library of molecular weights, such as, for example using database search software provided by the UK Human Genome Mapping Project Resource Centre.

Using a combination of multiple quadrupoles the amino acid sequence of a protein or peptide is determined.

Alternatively, the mass of a molecular ion is analysed using an ion trap mass analyser, essentially as described in Cooks et al, Chem. Eng. News, 69, 26, 1991. This form of analysis is a form of a quadrupole mass analyser where the generators of an electric charge are arrayed in three dimensions rather than in a linear fashion. In accordance with this embodiment, a molecular ion of a m/z ratio is trapped in a three-dimensional electric field. An ion trap mass analyser is also useful for in tandem mass spectrometry (MS/MS) experiments for the determination of a sequence of a peptide, polypeptide or protein. Methods of MS/MS are known in the art and/or described herein.

Alternatively or in addition, the mass of a molecular ion is analysed by its time of flight (TOF), essentially as described by Yates, J. Mass Spectrom. 33, 1-19, 1998 and references cited therein A time of flight instrument measures the m/z ratio of an ion by determining the time required for it to traverse the length of a flight tube. Optionally, such a TOF mass analyser includes an ion mirror at one end of the flight tube that reflects said ion back through the flight tube to a detector. Accordingly, an ion mirror serves to increase the length of a flight tube, increasing the accuracy of this form of analysis.

Time of flight analysis is also useful for determining the mass and therefore the predicted sequence of a peptide, polypeptide or protein.

Fourier transform ion cyclotron mass spectrometry, essentially as described in U.S. Pat. No. 3,937,955 is also useful in the analysis and identification of an immunogenic protein or fragment thereof isolated using the methods of the present invention. An ion cyclotron uses a fixed magnetic field to deflect an ion of known mass moving at a velocity through the field. Should the magnetic field strength be known, measurements of the ion cyclotron frequency suffices to determine the m/z ratio, ie., in a static magnetic field the mass-to-change ratio is uniquely determined by the ion-cyclotron frequency. In effect, the static magnetic field converts ionic mass into a frequency analogue.

As will be apparent from the exemplified subject matter, a mass spectrometer is useful for determining the amino acid sequence of a peptide, polypeptide or protein, using, for example, MS/MS. For example, an ion of interest (ie. an ionised peptide or protein of interest) is passed into a chamber of a mass spectrometer (a “collision chamber”), where the ion interacts with a gas, such as, for example nitrogen or argon. This interaction with a gas causes fragmentation of an ion, eg., within the peptide backbone. Following cleavage of an ion, mass analysis of resulting fragments that differ in mass by a single amino acid from another fragment enables the determination of an amino acid sequence of the ion of interest, ie., by determining the weight difference between peptides that differ by only one amino acid, the identity of each amino acid is determined. Mass spectrometers for the analysis of an amino acid sequence of a peptide, polypeptide or protein are, for example, a triple quadrupole (essentially as described in Hunt et al, Proc. Natl. Acad. Sci. USA, 83, 6233-6237, 1986), quadrupole-TOF (essentially as described in Morris et al, Rapid Commun. Mass Spectrom., 10, 889-896, 1996) or MALDI-QqTOF (essentially as described in Loboda et al, Rapid Commun. Mass Spectrom. 14, 1047-1057, 2000)

The sequence of several overlapping ions are, optionally, then be assembled, such that the sequence of a region, or even an entire polypeptide or protein is determined. Alternatively, the sequence of each individual ion is be used in flier analysis.

Following determining the sequence of at least a peptide derived from an immunogenic protein, this sequence is compared to a database of sequences to determine whether or not the derived sequence is identical to or substantially identical to a known sequence. Such a database is available, for example at NCBI or ExPASY or Swiss-Prot. Furthermore, as a mass spectrometer also determines the mass of a peptide, polypeptide or protein, this information is also useful in identifying an immunogenic protein, such as, by comparison to a protein mass library, such as, for example, that provided by the UK Human Genome Mapping Project Resource Centre.

As used herein the term “ExPASY” shall be taken to mean the Expert Protein Analysis System at the Swiss Institute of Bioinformatics at Basel University 4056, Basel, Switzerland.

As used herein the term “Swiss-Prot” shall be taken to mean the protein sequence database of the Swiss Institute of Bioinformatics at Basel University 4056, Basel, Switzerland.

Biomolecular interaction analysis-mass spectrometry (BIA-MS) is also useful for detecting and/or characterise and/or identify an immunogenic protein bound to said immunoglobulin (Nelson et al. Electrophoresis 21: 1155-1163, 2000).

Alternatively, a protein isolated using the method of the present invention is identified using an antibody or ligand capable of specifically binding to the isolated protein. In this regard, an antibody and/or ligand chip is useful for rapid analysis for protein identification. For example, an antibody array (ie. a glass slide upon which 380 or 500 individual antibodies are immobilized each in a defined area and each in duplicate) is available from Clontech. To use such an array, an immunogenic protein or fragment thereof isolated with the method of the present invention (whether bound to an immunoglobulin-containing fraction or protein complex comprising an immunoglobulin or mixtures thereof) is labelled with a detectable marker, eg., a fluorescent label (eg., Cy3 or Cy5). The labelled protein is then contacted to the antibody array for a time and under conditions sufficient for an antigen-antibody interaction to occur. The array is then washed and any bound labelled protein detected. By determining which antibody a protein is bound to, the identity of the immunogenic protein is determined.

Identification of an Immunogenic Protein from an Agent that Causes a Disease or Disorder

The present invention also provides a method for identifying an immunogenic protein or immunogenic protein fragment of an agent that causes a disease or disorder in a subject comprising:
(i) obtaining a protein complex comprising an immunoglobulin or mixtures thereof or an immunoglobulin-containing fraction from a subject suffering from the disease or disorder or having suffered previously from the disease or disorder or a cell, tissue or organ thereof;
(ii) contacting immunoglobulin in the protein complex or immunoglobulin-containing fraction with a sample comprising the agent that causes the disease or disorder or a derivative thereof; and
(ii) identifying a protein or fragment thereof bound to said immunoglobulin by virtue of an antigen-antibody interaction,
wherein the identified protein is an immunogenic protein or immunogenic protein fragment of an agent that causes a disease or disorder in a subject

As will be apparent to the skilled artisan the subject method optionally also comprises obtaining a sample that comprises the protein complex or immunoglobulin-containing fraction from the subject. For example, the sample that comprises an immunoglobulin-containing fraction or a protein comprising an immunoglobulin-containing fraction or mixtures thereof. Suitable samples are described supra and are to be taken to apply mutatis mutandis to the instant method.

An immunoglobulin-containing fraction or a protein complex comprising an immunoglobulin or a mixture thereof is isolated or derived from the subject or sample using a method described herein, for example, by contacting the sample with one or more compounds capable of binding an immunoglobulin for a time and under conditions sufficient for binding to occur and isolating the compound.

Using one or more immunoglobulin binding compounds previously immobilized on a solid support, matrix or resin facilitates isolation of a protein complex comprising an immunoglobulin or mixtures thereof or an immunoglobulin-containing fraction and a protein or fragment thereof bound thereto. Furthermore, such immobilized immunoglobulin binding compounds facilitates washing of the isolated immobilized compound to remove any non-specifically bound or unbound protein.

For example, the method of the present invention is performed with one or more immunoglobulin binding compounds immobilized on a magnetic or paramagnetic bead. By contacting said bead with a biological sample, a protein complex comprising an immunoglobulin or mixtures thereof or an immunoglobulin-containing fraction is isolated by exposing the sample to a magnetic or paramagnetic field thereby isolating the bead and a protein bound thereto. The bead is then optionally washed to remove any non-specifically bound or unbound protein. This bead is then useful for capturing an immunogenic protein.

Alternatively, the immunoglobulin binding compounds are immobilized, for example, on agarose. As agarose is a relatively large molecule with a high molecular weight, it is readily isolated by centrifugation. Accordingly, a protein complex comprising an immunoglobulin or mixtures thereof or an immunoglobulin-containing fraction is isolated by contacting that agarose bound immunoglobulin binding compound with a biological sample, eg., a sample described herein. The sample is then centrifuged and the precipitated agarose collected. Optionally, the agarose is washed to remove any non-specifically bound or unbound protein and collected again. Such an agarose bound protein complex comprising an immunoglobulin or mixtures thereof or an immunoglobulin-containing fraction is then useful for subsequent contacting with a sample to capture an immunogenic protein.

As exemplified herein, the present inventors have used one or more immunoglobulin binding compound (eg. protein G or protein A) immobilized on Sepharose. The Sepharose bound immunoglobulin binding compound is contacted with a sample comprising a protein complex comprising an immunoglobulin or mixtures thereof or an immunoglobulin-containing fraction (eg. serum) for a time and under conditions sufficient for immunoglobulin binding to occur and then used to produce a column. This column facilitates washing of the immunoglobulin binding compound and/or subsequent contacting of a sample to the bound protein complex comprising an immunoglobulin or mixtures thereof or an immunoglobulin-containing fraction.

The immunoglobulin binding compound/s (eg., protein A and/or protein G and/or protein L) are optionally linked with the protein complex comprising an immunoglobulin or mixtures thereof or immunoglobulin-containing fraction using a compound and/or method known in the art, for example, those described supra.

Optionally, an immunogenic protein or fragment thereof is separated from an immunoglobulin containing fraction and/or isolated using a method known in the art and/or described supra. The immunogenic protein is then identified using a method known in the art and/or described supra.

As the subject from whom the protein complex comprising an immunoglobulin or mixtures thereof or the immunoglobulin-containing fraction is derived has previously or is currently suffering from the disease or disorder, said subject has developed antibodies to the agent that causes said disease or disorder. These antibodies (immunoglobulins) facilitate isolation of an immunogenic protein or fragment thereof form the agent that causes the disease or disorder.

Accordingly, by contacting said antibody (in the form of the protein complex or immunoglobulin-containing fraction) with the agent that causes the disease or disorder or an extract thereof, a protein or fragment thereof against which a subject has raised an immune response is isolated and/or identified.

As used herein, the term “an extract” of (or derived from) an agent that causes a disease or disorder shall be taken to mean a preparation of one or more components of said agent. For example, the term “extract” encompasses, a cell lysate, a cellular fraction of said agent (eg., a cell wall fraction, a membrane fraction, a cytoplasmic fraction, a nuclear fraction or a mitochondrial fraction) or a protein extract from the cell (eg., produced by lysing a cell and collecting a protein containing fraction using a method known in the art). Alternatively, or in addition, the term “extract” also encompasses a particular subset of cells (and/or extracts thereof) derived from the agent, for example, a population of cells from a cancer, or a population of cells from a pathogen, eg. a parasite.

The method of the present invention is useful for identifying a protein bound to an immunoglobulin by virtue of a conformational antigen (ie. an antigen formed by the three dimensional structure of a protein or fragment) and/or a linear antigen. For example, an extract of a cell is prepared in such a way as to preserve the structure of a protein contained therein, ie the cell extract is prepared under non-denaturing conditions. Such a method is useful for identifying either a conformational epitope or a linear epitope.

Alternatively, the sample is prepared under denaturing conditions. By “denatured” or “denaturing” is meant that conformational epitopes of the protein are disrupted under conditions that retain linear B cell epitopes of the protein. For example, a cell extract is heated to a temperature that disrupts intramolecular bonds or treated with an ionic detergent (for example sodium-dodecyl sulfate (SDS)), and/or with a dissociating agent (for example urea or mercaptoethanol).

In one example, the disease or disorder is an infectious disease or disorder, eg. a disease or disorder caused by an infection by an agent selected from the group consisting of a virus, a bacterium, a yeast, a fungus and a parasite (such infectious agents are described supra). Accordingly, the subject infected with such an agent has developed an immune response against the agent. By contacting the an immunoglobulin fraction or a protein complex comprising an immunoglobulin or mixtures thereof with the infectious agent or an extract derived therefrom an immunogenic protein from said agent is identified. Such an immunogenic protein is useful as a diagnostic and/or therapeutic marker of said agent.

As will be apparent to the skilled artisan, when using the method of the invention to determine an immunogenic protein from an infectious organism it may be advantageous to use a clinical isolate of said infectious agent, eg., a clinical isolate of a bacterium. Such an isolate is known to be associated with a disease and/or disorder and has been cultured (and/or isolated) to ensure that the infectious agent is substantially free of a contaminating agent. Using the method of the invention method, the present inventors have identified an immunogenic protein from a bacterium (ie. Mycobacterium tuberculosis) using an immunoglobulin fraction derived from a subject suffering from an infection by said bacterium.

The instant method is also useful for identifying an immunogenic protein from other agents that cause a disease or a disorder. For example, the method of the present invention is useful for determining an immunogenic protein from a cancer cell. For example, an immunoglobulin fraction or a protein complex comprising an immunoglobulin or a mixture thereof is derived or isolated from a subject suffering from a cancer and said fraction or complex is contacted with a cell that causes said cancer or an extract thereof. An immunogenic protein or a fragment thereof identified using the method of the present invention is then useful as a diagnostic/prognostic and/or therapeutic marker for said cancer cell. Examples of suitable cancer cells include a cancer cell selected from the group consisting of a bladder cancer cell, a breast cancer cell, a colorectal cancer cell, an endometrial cancer cell, a head and neck cancer cell, a leukemia cell, a lung cancer cell, a lymphoma cell, a melanoma cell, a non-small-cell lung cancer cell, an ovarian cancer cell, a prostate cancer cell, an acute lymphocytic leukemia cell, an adult acute myeloid leukemia cell, an adult non-Hodgkin's lymphoma cell, a brain tumor cell, a cervical cancer cell, a childhood sarcoma cell, a chronic lymphocytic leukemia cell, a chronic myeloid leukemia cell, an oesophageal cancer cell, a hairy cell leukemia cell, a kidney cancer cell, a liver cancer cell, a multiple myeloma cell, a neuroblastoma cell, an oral cancer cell, a pancreatic cancer cell, a primary central nervous system lymphoma cell, a skin cancer cell and a small-cell lung cancer cell.

As exemplified herein the present invention is useful for studying an ovarian cancer or a breast cancer to identify an immunogenic protein.

Identification of an Immunogenic Protein or Fragment thereof from an Autoimmune Condition

The invention additionally provides a method for identifying an immunogenic protein or fragment thereof from an autoimmune condition capable of eliciting an immune response in a subject, said method comprising:
(i) obtaining a protein complex comprising an immunoglobulin or mixtures thereof or an immunoglobulin-containing fraction from a subject suffering from an autoimmune condition or a cell, tissue or organ thereof:
(ii) contacting immunoglobulin in the protein complex or immunoglobulin-containing fraction with a sample comprising protein from a subject suffering from an autoimmune disease; and
(ii) identifying a protein or fragment thereof bound to said immunoglobulin by virtue of an antigen-antibody interaction,
   wherein the identified protein is an immunogenic protein or fragment thereof from an autoimmune condition capable of eliciting an immune response in a the subject.

As will be apparent to the skilled artisan, the subject method optionally additionally comprises obtaining a sample the comprises the protein complex or immunoglobulin-containing fraction from the subject. For example, the sample that comprises an immunoglobulin-containing fraction or a protein complex. Suitable samples and immunoglobulin fractions or protein complexes comprising an immunoglobulin or a mixture thereof are described supra and are to be taken to apply mutatis mutandis to the instant method.

For example, a sample is derived from a subject suffering from an autoimmune disease selected from the group consisting of Hashimoto's disease, systemic lupus erythematosus, Sjögren's disease, antiphospholipid syndrome, primary biliary cirrhosis, mixed connective tissue disease, chronic active hepatitis, Graves' disease, type I diabetes, rheumatoid arthritis, scleroderma, myasthenia gravis, multiple sclerosis and chronic idiopathic thrombocytopenic purpura.

The immunoglobulin-containing fraction or a protein complex is isolated or derived from the subject or sample using a method described herein, for example, by contacting the sample with one or more compounds capable of binding an immunoglobulin for a time and under conditions sufficient for binding to occur and isolating the compound. A method and/or compound for isolating an immunoglobulin-containing fraction or a protein comprising an immunoglobulin or mixtures thereof is described supra and is to be taken to apply mutatis mutandis to the instant method.

The immunoglobulin binding compound/s (eg., protein A and/or protein G and/or protein L) are optionally linked with the protein complex or immunoglobulin-containing fraction using a compound and/or method known in the art, for example, those described supra.

As the subject suffers from an autoimmune condition, they have produced or developed antibodies that specifically recognise a self-antigen (eg., a protein or fragment thereof). By “self-antigen” is meant that the subject has developed an antibody that is capable of binding to an antigen (eg., a protein or a fragment thereof), wherein said antigen also occurs within or is produced by the subject. Such an antibody (in the form of an immunoglobulin-containing fraction or a protein complex comprising an immunoglobulin or mixtures thereof is useful for isolating and/or identifying an immunogenic protein or protein fragment against which the subject has developed an immune response. Such an immunogenic protein or fragment represents an attractive therapeutic and/or diagnostic/prognostic target for the autoimmune disease.

The present invention is useful for isolating and/or determining an immunogenic protein from an autoimmune disease. For example, an autoimmune disease is selected from the group consisting of rheumatoid arthritis, multiple sclerosis, type-1 diabetes, inflammatory bowel disease, Crohn's Disease, ulcerative colitis, systemic lupus erythematosus, psoriasis, scleroderma, autoimmune thyroid disease, central nervous system vasculitis, and autoimmune myositis. The clinical presentation of such a disease or disorder is caused by the autoimmune response of a subject to one or more self-antigens. For example, an autoimmune response against pancreatic islet cells causes these cells to be killed, thereby suppressing production of insulin and causing type-I diabetes.

The present invention is also useful for isolating and/or identifying an immunogenic protein from an autoimmune component from a disease or disorder. For example, a subject suffering from an inflammatory condition develops autoantibodies that bind to one or more proteins that are a components of the inflammatory response.

Accordingly, the method of the present invention is useful for determining an immunogenic protein or fragment thereof against which such a subject has developed an antibody. For example, the present inventors have used the method of the invention to identify an immunogenic protein in a subject that suffers from cystic fibrosis. The subject in question previously suffered from and/or was suffering from an acute clinical exacerbation, for example, an acute clinical exacerbation caused by an infection with a bacterium (eg., a bacterium selected from the group consisting of Staphylococcus aureus, Pseudomonas aeruginosa, Haemophilus influenzae, Aspergillus fumigatus, Burkholderia cepacia complex, Stenotrophomonas maltophila, Alcaligenes (Achromobacter) xylosoxidans, B. gladioli and Ralstonia picketti). Such an acute clinical exacerbation is characterised by an inflammatory response, that has been shown to be associated with development of an autoimmune response in some CF subjects.

The sample with which the immunoglobulin-containing fraction or protein complex comprising an immunoglobulin or mixtures thereof is contacted with a biological sample that comprises an immunogenic protein or fragment thereof, for example, a biological sample described supra. For example, such a biological sample is derived from a subject suffering from an autoimmune condition, eg., a subject suffering from the same autoimmune condition as the subject from whom the immunoglobulin-containing fraction or protein complex comprising an immunoglobulin or mixtures thereof is derived or isolated. For example, the immunoglobulin-containing fraction or protein complex comprising an immunoglobulin or mixtures thereof and the biological sample are derived from the same subject.

As exemplified herein, the present inventors have derived an immunoglobulin-containing fraction or protein complex comprising an immunoglobulin or mixtures thereof from one or more subjects suffering from an acute clinical exacerbation and contacted this sample with a sputum sample derived from one or more CF subjects suffering from an acute clinical exacerbation. Using this method a number of proteins have been isolated against which a CF subject suffering from an acute clinical exacerbation has raised an immune response.

Alternatively, the sample comprises a cell or extract thereof that comprises a protein or fragment thereof that the subject is suspected of raising an immune response against. For example, an immunoglobulin-containing fraction or protein complex comprising an immunoglobulin or mixtures thereof derived from a subject suffering from type-I diabetes is contacted with a pancreatic islet cell or an extract thereof.

Optionally, an immunogenic protein or fragment thereof is separated from an immunoglobulin containing fraction and/or isolated using a method known in the art and/or described supra. The immunogenic protein is then identified using a method known in the art and/or described supra.

Immunization of a Subject to Identify an Immunogenic Protein or Fragment Thereof

The present invention additionally provides a method for identifying an immunogenic protein or fragment thereof capable of eliciting an immune response in a subject, said method comprising:
(i) obtaining a protein complex comprising an immunoglobulin or mixtures thereof or an immunoglobulin-containing fraction from a sample from or produced by a subject previously administered with a sample comprising a cell or cell extract or mixture thereof comprising the immunogenic protein or fragment thereof;
(ii) contacting the protein complex or immunoglobulin-containing fraction with a sample comprising the cell or cell extract or mixture thereof; and
(iii) identifying a protein or fragment thereof bound to immunoglobulin in the protein complex or immunoglobulin-containing fraction by virtue of an antigen antibody interaction,
thereby identifying an immunogenic protein or fragment thereof capable of eliciting an immune response in a subject.

Optionally, the method additionally comprises administering the cell or cell extract to the subject. For example, the subject is immunized with the cell or cell extract. Methods for administering a cell or cell extract to a subject are known in the art. For example, the cell or cell extract is administered orally, by inhalation, by transdermal administration, topical administration or by injection (eg., intraperitoneal injection, intramuscular injection, subcutaneous injection or intravenous injection or infusion). As exemplified herein, a cell or cell extract or mixtures thereof administered by injection is useful for inducing production of immunoglobulin against said cell or cell extract or mixtures thereof

A suitable subject to whom the cell or cell lysate is administered is described supra and includes, for example, a subject selected from the group consisting of mouse, rat, rabbit, chicken, dog, sheep, ovine, horse and goat. As exemplified herein, the present inventors have immunized chickens to induce an immune response against a cell extract and identified an immunogen protein from that extract. Furthermore, as described in the examples, mice are useful for the method of the present invention, eg. mice are immunized with a cell or cell extract and immunoglobulin is isolated from, for example serum from the immunized mouse.

As will be apparent to the skilled artisan the cell or cell extract or mixtures thereof is, for example, administered to a subject and the subject allowed sufficient time to produce immunoglobulin that binds to said cell or cell extract or mixtures thereof. Optionally, the subject is administered or immunized with the cell or cell extract according to a predetermined schedule incorporating one or more booster immunizations or administrations. Such a schedule aids in the production of a stronger antibody (ie. immunoglobulin) response to the cell or cell extract or mixtures thereof.

Optionally, the method of the invention additionally comprises determining a subject that has produced immunoglobulin capable of binding an immunogenic protein in a cell or cell extract or mixtures thereof. Methods for determining the presence of an antibody or immunoglobulin in a sample are known in the art and include, for example, an enzyme-linked immunosorbent assay (ELISA), a radioimmunoassay (RIA) or modifications thereof, biosensor technology or evanescent fibre-optics technology amongst others.

For example, a standard solid-phase ELISA format is useful for determining the presence of an immunoglobulin capable of binding to a cell or cell extract in a sample derived from or produced by a subject.

In one form, such as an assay involves immobilising the cell or cell extract onto a solid matrix, such as, for example, a polystyrene or polycarbonate microwell or dipstick, a membrane, or a glass support (e.g. a glass slide).

A sample derived from or produced by a subject is brought into direct contact with the immobilised biological sample, and any immunoglobulin capable of binding said cell or cell extract forms a direct bond with any of its target protein present in said sample.

The bound immunoglobulin is then detected using a labelled antibody. Suitable labels include, for example, a fluorescent label (e.g. FITC or Texas Red), a fluorescent semiconductor nanocrystal (as described in U.S. Pat. No. 6,306,610) or an enzyme (e.g. horseradish peroxidase (HRP)), alkaline phosphatase (AP) or β-galactosidase. For example, an immunoglobulin captured or isolated from a chicken sample is detected using an anti-chicken antibody. Alternatively, a third labelled antibody can be used that binds the second (detecting) antibody. Following washing to remove any unbound antibody, the label is detected either directly, in the case of a fluorescent label, or through the addition of a suitable substrate, such as for example hydrogen peroxide, TMB, toluidine, or 5-bromo-4-chloro-3-indol-beta-D-galaotopyranoside (x-gal). Suitable substrates will depend upon the reporter molecule used, and will be apparent to the skilled artisan.

Suitable cells or cell extracts are described supra and include, for example, an agent that causes a disease or disorder, eg., infectious organism (eg., an organism selected from the group consisting of a virus, a bacterium, a yeast, a fungus and a parasite) or a cancer cell (eg., an ovarian cancer cell or a breast cancer cell). Such agents are described supra and are to be taken to apply mutatis mutandis to the instant method.

As the method of the invention utilizes an immunoglobulin that has been raised against an antigen in or on the cell and/or cell extract, said cell and/or cell extract is optionally administered with an agent or compound that enhances an immune response. For example, the cell or cell extract is administered with an adjuvant to increase the immune response to the cell or cell extract. An adjuvant that is used to increase the immunological response depends on the host species and include, for example, Freund's adjuvant (complete or incomplete), mineral gels such as aluminium hydroxide, surface active substances such as lysolecithin, pluronic polyols, polyanions, peptides, oil emulsions, keyhole limpet hemocyanin, and dinitrophenol. Other potentially useful adjuvants include BCG (bacille Calmette-Guerin) and Corynebacterium parvum.

As will be apparent to the skilled artisan a cell or cell extract is optionally administered in the form of a composition. For example, an appropriate composition comprising the cell or cell extract to be administered can be prepared in a physiologically acceptable vehicle or carrier. For solutions or emulsions, suitable carriers include, for example, aqueous or alcoholic/aqueous solutions, emulsions or suspensions, including saline and buffered media. Parenteral vehicles can include sodium chloride solution, Ringer's dextrose, dextrose and sodium chloride, lactated Ringer's or fixed oils, for instance. Intravenous vehicles can include various additives, preservatives, or fluid, nutrient or electrolyte replenishers and the like (See, generally, Remington's Pharmaceutical Sciences, 17th Edition, Mack Publishing Co., Pa., 1985). For inhalation, the agent can be solubilized and loaded into a suitable dispenser for administration (e.g., an atomizer, nebulizer or pressurized aerosol dispenser). As described supra, such a composition optionally includes an adjuvant.

As will be apparent to the skilled artisan, the subject method optionally additionally comprises obtaining a sample derived from the subject. For example, the sample that comprises an immunoglobulin-containing fraction or a protein comprising an immunoglobulin or mixtures thereof. Suitable samples and immunoglobulin fractions or protein complexes comprising an immunoglobulin or a mixture thereof are described supra and are to be taken to apply mutatis mutandis to the instant method.

Alternatively, a sample from which a protein complex or immunoglobulin containing fraction is isolated is produced by the subject. For example, a sample produced by a subject includes, an egg produced by an avian species, an embryo or a foetus. For example, the present inventors have immunized a chicken with a cell or cell extract, collected eggs produced by that chicken and isolated a protein comprising an immunoglobulin or mixtures thereof or an immunoglobulin containing fraction thereof from an egg.

A suitable subject for isolation of a protein complex or immunoglobulin containing fraction from a sample produced therefrom is from the class Aves. All birds are contemplated (e.g., duck, ostrich, emu, turkey, chicken, amongst others.). A preferred bird is a chicken. Methods for producing immunoglobulin in an avian species, eg., a chicken, are known in the art and described, for example, in A. A. Benedict and K. Yamaqa, Comparative Immunology, (J. J. Marchaloni, ed.), Ch. 13, “Immunoglobulins and Antibody Production in Avian Species,” pp. 335-375, Blackwell, Oxford (1966).

As laying hens export immunoglobulin to the egg yolk (eg., IgY) in concentrations equal to or exceeding that found in serum. (R. Patterson et al., J. Immunol., 89:272 1962), eggs represent an attractive source for isolation of a protein comprising an immunoglobulin or mixtures thereof or an immunoglobulin containing fraction thereof. Methods for isolating immunoglobulin from an egg are known in the art and, generally comprise separation of the yolk from the white using, for example, mechanical means or electrophoresis. Yolks are then optionally disrupted and diluted and a protein comprising an immunoglobulin or mixtures thereof or an immunoglobulin containing fraction thereof is isolated using a method described supra. As will be apparent to the skilled artisan, such a method isolates IgY.

Alternatively, a protein comprising an immunoglobulin or mixtures thereof or an immunoglobulin containing fraction thereof is isolated using the method described in USSN 20020028917.

An immunoglobulin-containing fraction or a protein complex is isolated or derived from the subject or a sample from or produced by the subject using a method described herein, for example, by contacting the sample with one or more compounds capable of binding an immunoglobulin-containing fraction for a time and under conditions sufficient for binding to occur and isolating the compound with immunoglobulin bound thereto. A method and/or compound for isolating an immunoglobulin-containing fraction or a protein comprising an immunoglobulin or mixtures thereof is described supra and is to be taken to apply mutatis mutandis to the instant method.

The immunoglobulin binding compound/s (eg., protein A and/or protein G and/or protein L) are optionally linked with the protein complex comprising an immunoglobulin or mixtures thereof or immunoglobulin-containing fraction using a compound and/or method known in the art, for example, those described supra.

Optionally, an immunogenic protein or fragment thereof is separated from an immunoglobulin containing fraction and/or isolated using a method known in the art and/or described supra. The immunogenic protein is then identified using a method known in the art and/or described supra.

The present method is useful for, for example, determining an immunogenic protein from an agent that causes a disease or disorder for use as a diagnostic/prognostic or therapeutic of said disease or disorder. For example, the method is useful for determining an immunogenic protein from an agent that causes a disease or disorder using a non-human animal as the subject that is immunized.

Alternatively, the method is useful for identifying an immunogenic protein from any cell or cell extract that comprises a protein or fragment thereof that is capable of eliciting an immune response in a subject. For example, the method is useful for identifying an immunogenic protein from a cancer cell or a cell against which a subject has raised an autoimmune response.

In one example of the method, the sample comprising the cell or cell extract or mixture thereof that is contacted to the protein complex comprising an immunoglobulin or mixtures thereof or an immunoglobulin-containing fraction thereof is derived from a subject comprising the cell or cell extract. For example, the cell or cell extract is derived from an agent that causes a disease or disorder and the sample comprising the cell or cell extract or mixture thereof is derived from a subject suffering from the disease or disorder. Accordingly, an immunoglobulin fraction is isolated from a sample derived from or produced by a subject immunized with a cell or cell extract and said immunoglobulin fraction is used to isolate an immunogenic protein or fragment form a subject that comprises the cell or cell extract. The immunized subject and the subject comprising the cell or cell extract are optionally not the same subject. For example, the immunoglobulin fraction is isolated from an egg of a chicken immunized with a cell or cell extract and then used to identify an immunogenic protein in a sample derived from a human subject that comprises the cell or cell extract.

Such a method is useful for, for example identifying an immunogenic protein from ane agent that causes a disease or disorder is an infectious agent, eg., an infectious agent is selected from the group consisting of a virus, a bacterium (eg., Mycobacterium tuberculosis), a yeast, a fungus and a parasite.

Increasing the Number and/or Amount of Immunogenic Proteins Identified

The method of the present invention is useful for identifying an immunogenic protein from any of a variety of sources, such as, for example, an agent that causes a disease or disorder or an autoimmune disease. The present inventors have additionally found that by repetitively separating an immunogenic protein or fragment thereof from an immunoglobulin fraction or a protein complex comprising an immunoglobulin or mixtures thereof and contacting the immunoglobulin fraction with a biological sample (eg. the sample with which it was originally contacted) the number of proteins identified and/or the amount of protein recovered is increased.

Accordingly, the present invention additionally provides a method comprising:
(a) obtaining a protein complex comprising an immunoglobulin or mixtures thereof or an immunoglobulin-containing fraction from a subject that has raised an immune response against an immunogenic protein or fragment thereof or a cell, tissue or organ thereof by a method comprising contacting a sample from the subject with one or more compounds capable of binding an immunoglobulin for a time and under conditions sufficient for binding to occur and isolating the one or more compounds;
(b) linking immunoglobulin in the protein complex or immunoglobulin-containing fraction to the one or more compounds;
(c) separating an immunogenic protein or fragment thereof from the linked immunoglobulin;
(d) contacting a sample comprising the immunogenic protein or fragment thereof with the linked immunoglobulin;
(e) separating the immunogenic protein or fragment thereof from the linked immunoglobulin;
(f) optionally, repeating (d) and (e) one or more times; and
(g) identifying a protein or fragment thereof separated from the immunoglobulin,
thereby identifying an immunogenic protein or fragment thereof.

In another form, the method of the invention provides a method comprising:
(a) obtaining a protein complex comprising an immunoglobulin or mixtures thereof or an immunoglobulin-containing fraction from a sample produced by a subject that has raised an immune response against an immunogenic protein or fragment thereof by a method comprising contacting a sample from the subject with one or more compounds capable of binding an immunoglobulin for a time and under conditions sufficient for binding to occur and isolating the one or more compounds;
(b) linking immunoglobulin in the protein complex or immunoglobulin-containing fraction to the one or more compounds;
(c) separating an immunogenic protein or fragment thereof from the linked immunoglobulin;
(d) contacting a sample comprising the immunogenic protein or fragment thereof with the linked immunoglobulin;
(e) separating the immunogenic protein or fragment thereof from the linked immunoglobulin;
(f) optionally, repeating (d) and (e) one or more times; and
(g) identifying a protein or fragment thereof separated from the immunoglobulin,
thereby identifying an immunogenic protein or fragment thereof.

In one form of the method (e) separating the immunogenic protein or fragment thereof from the linked immunoglobulin is performed prior to (d) contacting a sample comprising the immunogenic protein or fragment thereof with the linked immunoglobulin.

In another form of the invention (d) contacting a sample comprising the immunogenic protein or fragment thereof with the linked immunoglobulin is performed prior to (e) separating the immunogenic protein or fragment thereof from the linked immunoglobulin.

Preferably, (d) contacting a sample comprising the immunogenic protein or fragment thereof with the linked immunoglobulin and (e) separating the immunogenic protein or fragment thereof from the linked immunoglobulin are repeated a sufficient number of times to identify one or more immunogenic proteins. For example, (d) contacting a sample comprising the immunogenic protein or fragment thereof with the linked immunoglobulin and (e) separating the immunogenic protein or fragment thereof from the linked immunoglobulin are repeated a sufficient number of times to distinguish one or more proteins or fragments thereof on a gel using gel electrophoresis, for example, two-dimensional gel electrophoresis.

In one example of the method, the subject has raised an immune response against an agent that causes a disease or disorder. In accordance with this example, the sample comprising the immunogenic protein or fragment thereof that is contacted with the linked immunoglobulin comprises the agent that causes the disease or disorder or a derivative thereof.

For example, the agent that causes the disease or disorder is an infectious agent, eg., a bacterium, eg., Mycobacterium tuberculosis.

In another example, the subject suffers from an autoimmune condition. In accordance with this example, the sample comprising the immunogenic protein or fragment thereof that is contacted with the linked immunoglobulin comprises protein from a subject suffering from an autoimmune condition.

In yet another example, the subject has been previously immunized with a sample comprising a cell or extract thereof or mixtures thereof comprising the immunogenic protein or fragment thereof. In accordance with this example the sample comprising the immunogenic protein or fragment thereof that is contacted with the linked immunoglobulin comprises the cell or extract thereof. In one form, the subject is a chicken.

In an exemplified form of the method the subject has been previously immunized with a cell or cell extract from an agent associated with a disease or disorder, eg., an infectious agent, eg., a bacterium. In one example, the bacterium is Mycobacterium tuberculosis.

As exemplified herein, the process of repeating separating the immunogenic protein or fragment thereof and the immunoglobulin in the protein complex comprising an immunoglobulin or mixtures thereof or the immunoglobulin fraction is repeated until an immunogenic protein is identified. Alternatively, or in addition, the process is repeated until a sufficient number of immunogenic proteins or immunogenic protein fragments are identified.

As will be apparent from the preceding discussion, the method of the invention comprises eluting or separating an immunogenic protein or fragment from an immunoglobulin fraction and re-contacting the immunoglobulin fraction with a biological sample. This method is repeated as many times as required to identify an immunogenic protein. For example, the method is repeated at least 2 times, or three times, or four times, or five times, or six times, or seven times, or eight times or nin times. For example, each of the samples comprising an immunogenic protein or fragment eluted or separated from the immunoglobulin fraction is combined or pooled, thereby increasing the level of protein in the sample. Optionally, the process of the invention additionally comprises concentrating such a pooled sample.

By repetitively capturing and eluting immunogenic protein or fragments from a sample, increasing levels of said proteins or fragments are isolated facilitating identification of an immunogenic protein or fragment.

Furthermore, the present inventors have found, that by repetitively separating an immunogenic protein from an immunoglobulin and contacting the immunoglobulin with a sample, eg., from an infectious organism, a profile of proteins (eg., as determined using gel electrophoresis, eg., 2-dimensional gel electrophoresis) that substantially resembles the profile from the infectious organism is obtained.

Optionally, a method of the invention additionally comprises isolating a protein that was bound to the immunoglobulin-containing fraction by virtue of an antigen-antibody interaction, for example, by gel electrophoresis, eg. two-dimensional gel electrophoresis. Accordingly, it is preferable, that the step of repeatedly contacting an immunoglobulin fraction with a biological sample and separating a bound protein is repeated a sufficient number of time to distinguish one or more immunogenic proteins using gel electrophoresis or two-dimensional gel electrophoresis.

Methods for identifying a protein isolated using the method of the present invention are known in the art and/or described supra, for example, matrix-assisted laser desorption/ionisation-time-of-flight mass spectrometry (MALDI-TOF MS).

Using the process described supra the present inventors have identified a number of proteins from an infectious organism associated with a human disease and from an autoimmune condition.

Furthermore, the present inventors have shown that by repeating the process of contacting an immunoglobulin fraction with a biological sample, both the number of proteins observed on a gel and identified and the amount of each protein is increased. Clearly the process described herein provides an advantage in amplifying the amount and number of target proteins or fragments thereof identified from a sample.

Uses for a Protein of Identified Using the Method of the Invention

As the method of the invention is useful for identifying an immunogenic protein or fragment of a protein, the method identifies a marker of a disease or a disorder useful for diagnosis/prognosis of a disease or disorder or a therapeutic of a disease or disorder.

Accordingly, the present invention provides for the use of the method of the invention in a process for identifying a marker of a condition.

Furthermore, the invention provides for the use of the method of the invention in the diagnosis of a condition, such as, for example, a disease or disorder, eg an infectious disease or a cancer or an autoimmune condition.

For example, the present inventors have shown that antibodies to the Pseudomonas aeruginosa protein GroES are detectable in a subject suffering from a P. aeruginosa infection, whereas such antibodies are not present in a healthy control subject.

The present invention additionally provides for a method of treatment or prophylaxis comprising performing the method of the present invention and identifying an immunogenic proteins from an agent associated with a disease or disorder and administering an effective amount of a compound for the treatment of said disease or disorder.

Alternatively an immunogenic protein or fragment thereof may be used as a form of prophylactic therapy, eg. as an antigen in a vaccine composition.

Accordingly, the present invention additionally provides a method for producing a vaccine comprising performing a method for identifying an immunogenic protein or fragment from an agent associated with a disease or disorder and manufacturing a vaccine comprising the immunogenic protein or fragment.

Such a vaccine comprises, for example, an adjuvant. A suitable adjuvant is known in the art and/or described herein. In one form of the invention, the vaccine is a composition comprising the identified immunogenic protein or fragment and, optionally, an adjuvant. Constituents of such a composition are known in the art and/or described herein.

The present invention additionally provides a method of manufacturing a compound or composition for the diagnosis or treatment or prophylaxis of a condition comprising:
(i) determining an immunogenic protein or fragment thereof using a method described herein; and
(ii) using the immunogenic protein or fragment thereof in the manufacture of a compound for the diagnosis or prophylaxis or treatment of the condition.

For example, the method comprises the additional step of isolating the immunogenic protein or fragment thereof.

The present invention clearly encompasses the use of any in silico analytical method and/or industrial process for carrying the screening methods described herein into a pilot scale production or industrial scale production of a compound or composition identified in such screens. This invention also provides for the provision of information for any such production. Accordingly, the present invention additionally provides a process for identifying or determining an immunogenic protein or fragment or composition supra, said method comprising:
(i) performing a method as described herein to thereby identify an immunogenic protein or fragment;
(ii) optionally, determining the structure of the protein or fragment thereof; and
(iii) providing the protein or fragment thereof or a composition comprising said protein or fragment thereof or the name or structure of the protein or fragment thereof or a composition comprising said protein or fragment thereof such as, for example, in a paper form, machine-readable form, or computer-readable form.

Naturally, for proteins, fragments or compositions that are known albeit not previously tested for their function using a screen provided of the present invention, determination of the structure of the compound is implicit in step (i) supra. This is because the skilled artisan will be aware of the name and/or structure of the compound at the time of performing the screen.

As used herein, the term “providing the immunogenic protein or fragment or composition” shall be taken to include any chemical and/or recombinant and/or synthetic means for producing said immunogenic protein or fragment or composition or alternatively, the provision of a immunogenic protein or fragment or composition that has been previously synthesized by any person or means.

In a preferred embodiment, the immunogenic protein or fragment or composition or the name or structure of the immunogenic protein or fragment or composition is provided with an indication as to its use e.g., as determined by a screen described herein.

The present invention additionally provides a process for producing a immunogenic protein or fragment or composition supra, said method comprising:

a process for identifying or determining an immunogenic protein or fragment or composition supra, said method comprising:

(i) performing a method as described herein to thereby identify or determine a immunogenic protein or fragment or composition for the diagnosis or treatment or prophylaxis of a condition;
(ii) optionally, determining the structure of the immunogenic protein or fragment or composition;
(iii) optionally, providing the name or structure of the immunogenic protein or fragment or composition such as, for example, in a paper form, machine-readable form, or computer-readable form; and
(iv) providing the immunogenic protein or fragment or composition.

In a preferred embodiment, the synthesized or produced immunogenic protein or fragment or composition or the name or structure of the immunogenic protein or fragment or composition is provided with an indication as to its use.

The invention additionally provides a method of manufacturing a immunogenic protein or fragment or composition for the diagnosis, treatment or prophylaxis or a condition comprising:
(i) determining a candidate immunogenic protein or fragment or composition for the diagnosis or treatment or prophylaxis of a condition; and
(ii) using the immunogenic protein or fragment or composition in the manufacture of a therapeutic or prophylactic or diagnostic for the treatment or diagnosis of a condition.

In one embodiment, the method comprises the additional step of isolating the candidate immunogenic protein or fragment or composition. Alternatively, a immunogenic protein or fragment or composition is identified and is produced for use in the manufacture of a immunogenic protein or fragment or composition for the diagnosis or treatment or prophylaxis of a condition.

The present invention is further described with reference to the following non-limiting examples.

### EXAMPLE 1

### Identification of M. tuberculosis Glutamine Synthetase in the Serum of a Tuberculosis Subject

1.1 Sample Preparation

1.5 ml of patient serum stored at −80° C. was thawed at room temperature then applied to a 2 ml column of protein G-SSepharose (Amersham Biosciences, Castle Hill, New South Wales, Australia), previously equilibrated with 20 mM phosphate buffer pH 7 and incubated on ice for 30 minutes with occasional inversion. The mixture was spun at 6000 g for 10 minutes at 4° C. and the supernatant decanted. The SSepharose pellet was washed with 20 mM phosphate buffer. The IgG bound to the SSepharose was eluted by addition of 50 mM glycine pH 2.7 for 20 minutes. After centrifugation as above, the supernatant was discarded and the glycine step repeated. The supernatant was collected from this second glycine elution and stored at −80° C.

A Bradford protein assay is performed on the thawed eluate and thirty milligrams of the immunoglobulin fraction loaded onto a Sephacryl S-200 high resolution gel filtration column (Amersham Biosciences). Fractions ranging from 3000 to 140000 MW are collected, excluding the 150000 IgG fraction. These fractions are pooled and precipitated with 10 volumes of cold acetone at −20° C. for 48 h then centrifuged at 5000 g for 20 mins at 4° C. The precipitates are resolubilised in 1-2 mls of sample buffer containing 5M urea, 2M thiourea, 2% CHAPS, 2% SB3-10 and 40 mM Tris, then simultaneously reduced with 5 mM tributyl phosphine and alkylated with 10 mM acrylamide for 1 h. Samples are aliquoted into 250 μl aliquots and stored at −80° C.

1.2 Two Dimensional Gel Electrophoresis of Samples

The protein content of the samples was estimated using a Bradford assay. Samples were diluted to 2 mg/ml with sample buffer as above replacing 40 mM Tris with 5 mM Tris.

Prior to rehydration of IPG strips, samples were centrifuged at 21000×g for 10 minutes. The supernatant was collected and 10 μl of 1% Orange G (Sigma) per ml added as an indicator dye.

First Dimension

Dry 11 cm IPG strips (Amersham-Biosciences) were rehydrated for 16-24 hours with 180 μl of protein sample. Rehydrated strips were focussed on a Protean IEF Cell (Bio-Rad, Hercules, Calif.) or Proteome System's IsoElectrIQ electrophoresis equipment for approx 140 kVhr at a maximum of 10 kV. Focussed strips were then equilibrated in urea/SDS/Tris-HCl/bromophenol blue buffer.

Second Dimension

Equilibrated strips were inserted into loading wells of 6-15% (w/v) tris-acetate SDS-PAGE pre-cast 10 cm×15 cm GelChips (Proteome Systems, Sydney Australia). Electrophoresis was performed at 50 mA per gel for 1.5 hours, or until the tracking dye reached the bottom of the gel. Proteins were stained using SyproRuby (Molecular Probes). Gel images were scanned after destaining using an AlphaImager System (Alpha Innotech Corp.). Gels were then stained with Coomassie G-250 to assist visualisation of protein spots in subsequent analyses.

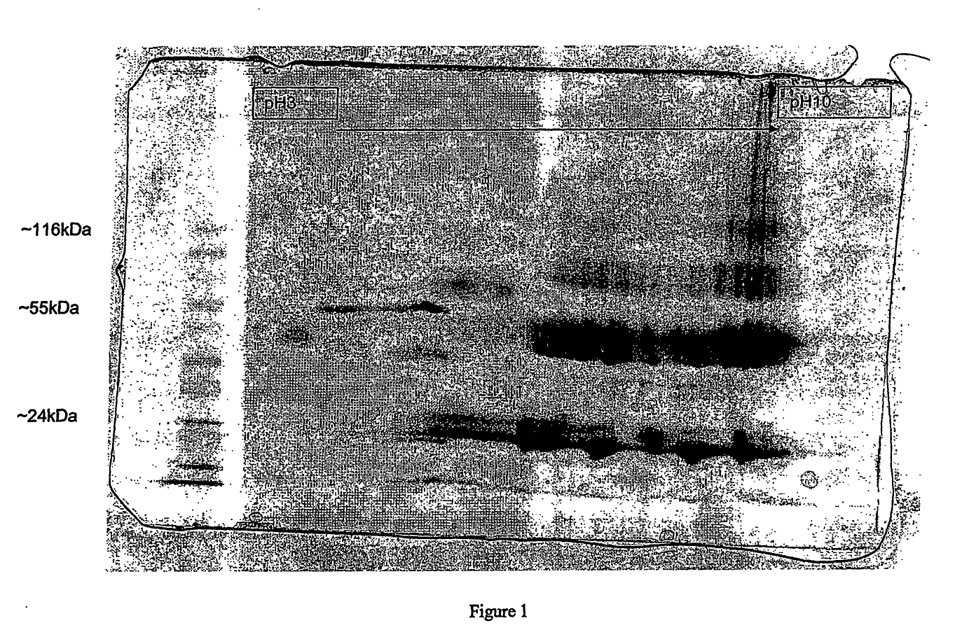

An example Gel Image is shown in FIG. 1.

1.3 Protein Identification

A number of proteins were observed in serum samples obtained from subjects suffering from a tuberculosis infection. These proteins were then identified using mass spectrometry.

Prior to mass spectrometry protein samples were prepared by in-gel tryptic digestion. Protein gel pieces were excised, destained, digested and desalted using an Xcise™, an excision/liquid handling robot (Proteome Systems, Sydney, Australia and Shimadzu-Biotech, Kyoto, Japan) in association with the Montage In-Gel Digestion Kit (developed by Proteome Systems and distributed by Millipore, Billerica, Mass., 01821, USA). Prior to spot cutting, the 2-D gel was incubated in water to maintain a constant size and prevent drying. Subsequently, the 2-D gel was placed on the Xcise, a digital image was captured and the spots to be cut were selected. After automated spot excision, gel pieces were subjected to automated liquid handling and in-gel digestion. Briefly, each spot was destained with 100 μl of 50% (v/v) acetonitrile in 50 mM ammonium bicarbonate. The gel pieces were dried by adding 100% acetonitrile, the acetonitrile was removed after 5 seconds and the gels were dried completely by evaporating the residual acetonitrile at 37° C. Proteolytic digestion was performed by rehydrating the dried gel pieces with 30 μl of 20 mM ammonium bicarbonate (pH 7.8) containing 5 μg/mL modified porcine trypsin and incubated at 30° C. overnight.

Ten μl of the tryptic peptide mixture was removed to a clean microtitre plate in the event that additional analysis by Liquid Chromatography (LC)-Electrospray Ionisation (ESI) MS was required.

Automated desalting and concentration of tryptic peptides prior to MALDI-TOF MS was performed using C18 ZipTip (Millipore, Bedford, Mass.). Adsorbed peptides were eluted from the tips onto a 384-position MALDI-TOF sample target plate (Kratos, Manchester, UK or Bruker Daltronics, Germany) using 2 μl of 2 mg/ml α-cyano-4-hydroxycinnamic acid in 90% (v/v) acetonitrile and 0.085% (v/v) TFA.

Digests were analysed using an Axima-CPR MALDI-TOF mass spectrometer (Kratos, Manchester, UK) in positive ion reflectron mode. A nitrogen laser with a wavelength of 337 nm was used to irradiate the sample. The spectra were acquired in automatic mode in the mass range 600 Da to 4000 Da applying a 64-point raster to each sample spot. Only spectra passing certain criteria were saved. All spectra underwent an internal two point calibration using an autodigested trypsin peak mass, m/z 842.51 Da and spiked adenocorticotropic hormone (ACTH) peptide, m/z 2465.117 Da Software designed by Proteome Systems, as contained in the web-based proteomic data management system BioinformatIQ™ (Proteome Systems), was used to extract isotopic peaks from MS spectra.

Protein identification was performed by matching the monoisotopic masses of the tryptic peptides (i.e. the peptide mass fingerprint) with the theoretical masses from protein databases using IonIQ database search software (Proteome System Limited, North Ryde, Sydney, Australia). Querying was done against the non-redundant SwissProt (Release 40) and TrEMBL (Release 20) databases (June 2002 version), and protein identities were ranked through a modification of the MOWSE scoring system. Propionamide-cysteine (cys-PAM) or carboxyamidomethyl-cysteine (cys-CAM) and oxidized methionine modifications were taken into account and a mass tolerance of 100 ppm was allowed.

Miscleavage sites were only considered after an initial search without miscleavages had been performed. The following criteria were used to evaluate the search results: the MOWSE score, the number and intensity of peptides matching the candidate protein, the coverage of the candidate protein's sequence by the matching peptides and the gel location.

In addition, or alternatively, proteins were analysed using LC-ESI-MS. Tryptic digest solutions of proteins (10 μl) were analysed by nanoflow LC/MS using an LCQ Deca Ion Trap mass spectrometer (ThermoFinnigan, San Jose, Calif.) equipped with a Surveyor LC system composed of an autosampler and pump. Peptides were separated using a PepFinder kit (Thermo-Finnigan) coupled to a C18 PicoFrit column (New Objective). Gradient elution from water containing 0.1% (v/v) formic acid (mobile phase A) to 90% (v/v) acetonitrile containing 0.1% (v/v) formic acid (mobile phase B) was performed over a 30-60-minute period. The mass spectrometer was set up to acquire three scan events—one full scan (range from 400 to 2000 amu) followed by two data dependant MS/MS scans.

Proteins were identified using SEQUEST (BioWorks 3.1, Thermo-Finnigan) software. Peptides were identified from MS/MS spectra in which more than half of the experimental fragment ions matched theoretical ion values, and gave cross-correlation (a raw correlation score of the top candidate peptide), delta correlation (difference in correlation between the top two candidate peptides) and preliminary score (raw score used to rank candidate peptides) values greater than 2.2, 0.2, and 400, respectively.

Using this method a 49.7 kDa protein was identified in the immunoglobulin fraction of a TB subject. This protein was analysed using MALDI-TOF MS and fragments (SEQ ID NO: 6-11) shown in Table 1 identified.

TABLE 1

Fragments identified from an
immunoglobulin associated protein

| | | | |
|---|---|---|---|
| Peptide | SEQ | Site of peptide | |
| Number | ID NO: | in protein | Sequence |

| | | | |
|---|---|---|---|
| 1 | 6 | 204-221 | FEAVKGECNMGQQEIGFR |
| 2 | 7 | 241-255 | EIADQHGKSLTFMAK |
| 3 | 8 | 305-318 | EFTLCYAPTINSYK |
| 4 | 9 | 343-351 | VVGHGQNIR |
| 5 | 10 | 401-419 | LPVTLADAAVLFEDSALVR |
| 6 | 11 | 436-450 | VELAAFNAAVTDWER |

Using this information sequence databases were searched and it was predicted that the isolated protein spot was a hypothetical M. tuberculosis glutamine synthetase protein (SEQ ID NO: 12). Clearly this demonstrates that this method is of use in the isolation and identification of proteins that are of particular use in the diagnosis/prognosis, treatment or prophylaxis of disorders such as infections or autoimmune diseases.

### EXAMPLE 2

### Alternate Method of Sample Preparation and Immunoglobulin Isolation

1.5 ml of patient serum stored at −80° C. was thawed at room temperature then applied to a 2 ml column of protein G-Sepharose (Amersham Biosciences), previously equilibrated with 20 mM phosphate buffer pH 7 and incubated on ice for 30 minutes with occasional inversion. The mixture was spun at 6000 g for 10 minutes at 4° C. and the supernatant decanted. The Sepharose pellet was washed with 20 mM phosphate buffer. The IgG bound to the Sepharose was eluted by addition of 50 mM glycine pH 2.7 for 20 minutes. After centrifugation as above, the supernatant was discarded and the glycine step repeated. The supernatant was collected from this second glycine elution and stored at −80° C.

Following sample preparation, proteins are separated using two-dimensional electrophoresis and immunogenic proteins identified using MALDI-TOF MS essentially as described in Example 1.

### EXAMPLE 3

### Isolation of an Immunogenic Protein from a Subject Suffering from TB

3.1 Sample Preparation

Serum (1.8 ml) from a patient infected with Mycobacterium tuberculosis and HIV was purified using a 1 ml protein A column attached to an ÄKTA Explorer (Amersham Biosciences). Briefly the sample was diluted in 8.2 ml Immunopure IgG binding buffer (Pierce cat number 21001), then filtered through a 0.221 μm filter before application to the column. Elution of bound antibody was with Immunopure gentle Ag/Ab elution buffer (Pierce cat number 21027). The eluted fractions were pooled (IgG bound to antigens) and left on ice for 3 hours to allow dissociation of immune complexes. The IgG fraction was separated from the antigen fraction by filtration through a 100,000 molecular weight cut off column (Millipore). Both fractions and the flow through from the protein A column were dialysed with benzoylated dialysis membrane (Sigma D-2272) against 4 litres of phosphate buffered saline pH 7.2 overnight at 4° C., then another 4 litres for 3 hours. All fractions, (flow through and retentate from the 100,000 cut off column and flow through from the protein A column), were acetone precipitated at a ratio of 10 parts acetone to 1 part sample for one hour at −20° C., then spun at 4000 g for 20 minutes. The precipitated samples were solubilized in sample buffer containing 5M urea, 2M thiourea, 2% CHAPS, 2% SB3-10 and 40 mM Tris to a final concentration of approx. 2 mg/ml, then simultaneously reduced with 5 mM tributyl phosphine and alkylated with 10 mM acrylamide for 1.5 h. The alkylation reaction was quenched with the addition of DTT to a final concentration of 10 mM. The samples were aliquoted into 200 μl lots and stored at −20° C.

2 μl of 1% orange G tracking dye was added to a 200 μl aliquot of each of the three fractions and centrifuged at 16,100 rcf for 20 mins.

3.2 Gel Electrophoresis

First Dimension

The supernatants were used to rehydrate dry Amersham Biosciences 11 cm pI 3-10 IPGs for approx. 24 h.

Rehydrated strips were focussed on a Protean IEF Cell (Bio-Rad, Hercules, Calif.) or Proteome System's IsoElectrIQ electrophoresis equipment for approx. 109500 Volt hours at a maximum of 10 kV. Focussed strips were then equilibrated in urea/SDS/Tris-HCl/bromophenol blue buffer.

Second Dimension

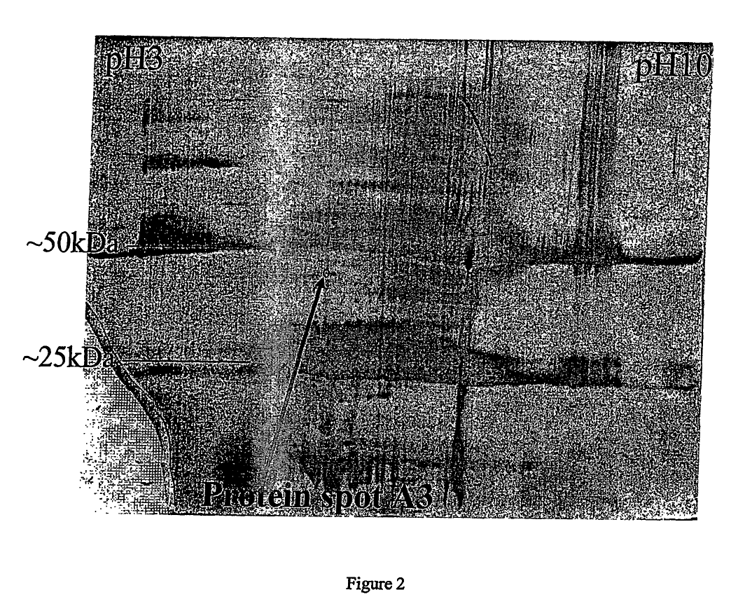

Equilibrated strips were inserted into loading wells of 6-15% (w/v) tris-acetate SDS-PAGE pre-cast 10 cm×15 cm GelChips (Proteome Systems, Sydney Australia). Electrophoresis was performed at 50 mA per gel for 1.5 hours, or until the tracking dye reached the bottom of the gel. The gels from the Retentate and Flow through fractions were stained using SyproRuby (Molecular Probes). Gel images were scanned after destaining using an AlphaImager System (Alpha Innotech Corp.). The gel from the Eluate fraction was stained with silver according to the protocol of Shevchenko et al. (Anal Chem. 1996 Mar. 1; 68(5): 850-8). The gel image was scanned using an UMAX flatbed scanner. An example gel is shown in FIG. 2, in which a protein having an isoelectric point of about 5.28 and a molecular weight of about 43590 Daltons was is marked. This protein spot was then further analysed.

3.3 Mass Spectrometry

Prior to mass spectrometry protein samples were prepared by in-gel tryptic digestion. Protein gel pieces from the Eluate fraction were excised manually and destained with 50 mM NH₄HCO₃/50% acetonitrile. 100 μl of this solution was added to each gel piece in a 96-well microtitre plate and placed on a shaking platform for 20 min. The destaining procedure was then repeated once. Gel pieces were dried in a 50° C. oven for 20 mins. 2.5 μl of 0.02 μg/l trypsin in 25 mM NH₄HCO₃ and 0.1% n-octyl-glycoside was then added to each dried gel piece. Samples were left on ice for 40 mins. 10 μl of 25 mM NH₄HCO₃/0.1% n-octyl-glycoside was then added to each well and the reaction left overnight at 30° C. 15 μl 0.1% TFA was added to each well and the plate sonicated in a sonic water bath for 15 mins. The TFA extraction and sonication steps were repeated once. A further 10 mins sonication was performed and 12 μl of sample solution containing tryptic peptides was transferred to a fresh plate.

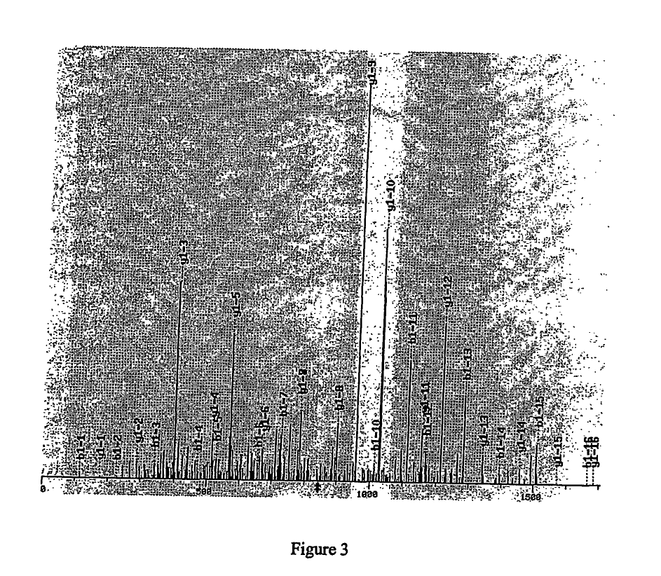

Proteins were analysed using LC-ESI-MS. Tryptic digest solutions of proteins (10 μl) were analysed by nanoflow LC/MS using an LCQ Deca Ion Trap mass spectrometer (ThermoFinnigan, San Jose, Calif.) equipped with a Surveyor LC system composed of an autosampler and pump. Peptides were separated using a PepFinder kit (Thermo-Finnigan) coupled to a C18 PicoFrit column (New Objective). Gradient elution from water containing 0.1% (v/v) formic acid (mobile phase A) to 90% (v/v) acetonitrile containing 0.1% (v/v) formic acid (mobile phase B) was performed over a 30 minute period. The mass spectrometer was set up to acquire three scan events—one full scan (range from 400 to 2000 amu) followed by two data dependant MS/MS scans. Using this method the mass spectrum of peptides of the protein spot identified previously was determined. The mass spectrum of such a peptide is shown in FIG. 3.

3.4 Protein Identification

Proteins were identified using SEQUEST (BioWorks 3.1, Thermo-Finnigan) software. Peptides were identified from MS/MS spectra in which more than half of the experimental fragment ions matched theoretical ion values, and gave cross-correlation (a raw correlation score of the top candidate peptide), delta correlation (difference in correlation between the top two candidate peptides) and preliminary score (raw score used to rank candidate peptides) values greater than 2.2, 0.2, and 400, respectively.

SEQUEST searching revealed a match to a peptide sequence (K)LLDQGQAGDNVGLLLR (SEQ ID NO: 15) (m/z 1682.92) (SwissProt accession number P31501).

Using this method a 43.59 kDa protein was identified in a TB subject. This protein was identified using LC-ESI-MS and the elongation factor-Tu protein of M. tuberculosis (SEQ ID NO: 14) was identified.

### EXAMPLE 4

### Identification of an Immunogenic Pathogen Derived Protein from a CF Subject

Subjects that suffer from cystic fibrosis are prone to infections by P. aeruginosa. To identify proteins from P. aeruginosa that may be useful in diagnosing such an infection immunoglobulin fraction was isolated from CF subjects and used to identify immunogenic proteins from the infectious bacterium.

4.1 Biological Samples

Crude plasma obtaine from whole blood samples from CF subjects. The crude plasma used from the capture column were combined from four exacerbated CF adults in the age group 22- to 37-years old. Predicted FEV₁ values were between 22-65% and the subjects have had 2-4 exacerbations in the last 12 months. All adult CF subjects used in current study had profuse P. aeruginosa growth in the lungs as shown by microbiological testing. In addition, one CF adult also had pulmonary S. aureus infection.

Saline-induced sputum was collected from healthy control- and CF subjects and subsequently liquefied. Resulting samples were pooled, alcohol precipitated and resolubilised in 7M urea, 2M thiourea, 2% chaps, 10 mM tris. Samples were reduced and alkylated with 5 mM TBP 10 mM acrylamide for 1 hr. Subsequently, samples were spun on 100 kda and 5 kda spin columns whereafter captured proteins arrayed by 2DE arrays

Sputum used for immuno-capturing: Sputum samples were pooled from two exacerbated CF subjects of 22 and 31 years old (total of 16 mL). They had predicted FEV₁ values of 14% and 51% and had been treated for 1-2 exacerbations in the last 12 months. Microbiological testing showed profuse P. aeruginosa in the lungs of both patients. In addition, one of the two patients also contained profuse S. aureus. The pooled sputum were incubated with 30 mM IAA to inactivate residual DTT used in the liquification protocol and IgG depleted by using Protein G coupled Sepharose beads as recommended by manufacturer (Amersham Pharmacia (Uppsala, Sweden).

4.2 Preparation of Proteins from P. aeruginosa

Overnight cultures of P. aeruginosa PA01 (200 mL ATCC culture) were pelleted by centrifugation (20 minutes at 4000 g, room temperature). The precipitated cells were washed twice in water and resuspended in Lysis Buffer A (50 mM Tris-HCl pH 7.6, 0.1 mM EDTA, 20% sucrose)+protease inhibitors (1× Complete Protease Inhibitor Cocktail, Roche Diagnostics, Basel, Switzerland). Cells were lysed using a Branson sonifier, model 250-450, using 70% of maximal amplitude for 4×10 seconds and unbroken cells were pelleted by centrifugation (4000 g, 10 min, 4° C.). Another centrifugation step was performed with the obtained supernatant prior acetone precipitation of proteins. Precipitated proteins were resolubilised in 10 mM PBS pH 7.2.

Membrane proteins: membrane proteins were extracted using the ProteoPrep membrane extraction kit essentially as recommended by manufacturer (Proteome Systems, Woburn, US). However, the resulting pellet after the last 50 mM Tris-HCl, pH 7.3 wash was resuspended in 10 mM PBS pH 7.4 containing 1% Triton-X, 15 mM Tris-HCl pH 7.5 and 20 mM DTT. After solubilisation, sample was incubated with 60 mM iodoacetamide for 2 hours at room temperature.

4.3 Preparation of an Immunocapture Column

An immuno-capture column was generated from a total of 5 mL pooled plasma from five exacerbated CF patients (total protein concentration of 40 mg/mL). IgG was bound to Protein G Sepharose by incubating the pooled plasma with 10 mL 50% slurry of Protein G Sepharose. The matrix were washed in 10 mM PBS pH 7.4 and bound IgG was irreversibly immobilised utilizing DSS.

4.4 Capture of an Immunogenic Protein from P. aeruginosa

The capture column was incubated overnight with the native P. aeruginosa protein extract (6.3) at 4° C. at constant rotation and beads were subsequently harvested by centrifugation. The flow-through was collected and saved for subsequent incubation steps (the protein extract was passed over the capture column three times in each capture). The harvested beads were washed 3 times in 10 mM PBS pH 7.4 and captured proteins were eluted with 50 mM glycine pH 2.7. The column was extensively washed with first 50 mM glycine pH 2.7 then 10 mM PBS pH 7.2 prior subsequent incubation steps.

Eluted proteins were alcohol precipitated (using ethanol in a ratio of 1:10) and subsequently resolubilised in Cellular and Organelle Membrane solubilizing reagent from the ProteoPrep Universal Extraction kit (Sigma, St. Louis, Mo.). Following the instruction in the ProteoPrep kit the solubilized proteins were reduced and alkylated with a final concentration of 5 mM tri-n-butylphophine and 10 mM acrylamide, respectively.

4.5 Two-Dimensional Gel Electrophoresis

Eleven centimetre pH 3-10 or pH 4-7 IPGs were purchased from Amersham (Uppsala, Sweden). Isoelectric focusing was conducted as per manufacturer's instructions using an IsoElectrIQ² unit from Proteome Systems (Woburn, Mass.). Second dimension 6-15% or 14% homogenous Tris-Acetate Gelchip gels were run as recommended by manufacturer (Proteome Systems, Woburn, Mass.). Arrayed proteins were visualised by silver-staining (Shevchenko et al., Anal Chem. 68, 850-858. 1996).

4.6 MS Analysis

Proteins spots of interest were excised and washed twice in 100 mM NH₄HCO₃: 50% acetonitrile (ACN) pH 8.2 and dehydrated at 50° C. for 30 minutes. Proteins were digested as described by Katayama et al Rapid Commun Mass Spectrom.; 15:1416-1421, 2001 and digested for 3 hours at 37° C. Tryptic peptides was extracted by sonication and purified as described by Kussmann et al. Peptides were eluted with ˜1.5 μl MALDI matrix solution (70% ACN, 0.1% TFA, 1.5 mg/ml alpha-cyano-4-hydroxycinnamic acid (Sigma, St. Louis, Mo.). Peptide mass fingerprints (PMF) were generated by matrix-assisted laser desorption/ionisation-time-of-flight-mass spectrometry (MALDI-TOF-MS) using an Axima CFR (Kratos, Manchester, UK) or an ABI MALDI MS/MS (AME Bioscience, London, UK).

4.7 Results

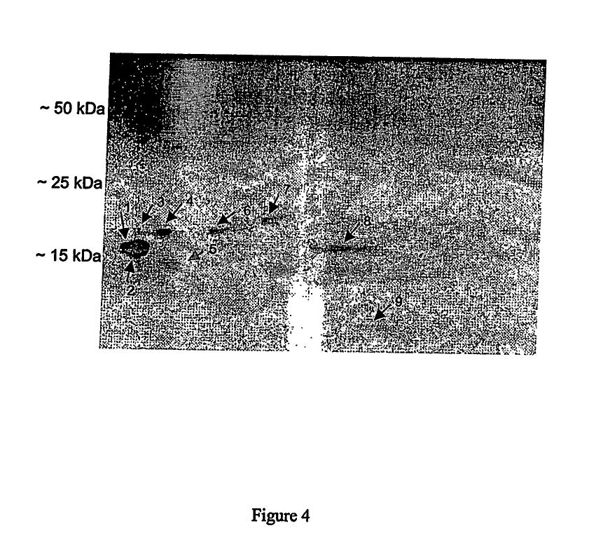

As shown in FIG. 4, six immunogenic proteins were captured from P. aeruginosa protein extracts using the immunocapture column. These proteins were analysed using MS. Following tryptic digestion the following peptides were identified using peptide mass fingerprinting:

TABLE 2

Peptides identified from an immunogenic
protein from M. tuberculosis

| | | | | | | | |
|---|---|---|---|---|---|---|---|
| Peptide | theoretical | | AA | | | | SEQ |
| mass | mass | error | (start) | AA | MC | peptide sequence | ID NO: |

| | | | | | | | |
|---|---|---|---|---|---|---|---|
| 905.488 | 905.519 | −0.031 | 3 | 9 | 0 | LRPLHDR | 16 |
| 943.539 | 943.508 | 0.031 | 38 | 47 | 0 | GEVVAVGTGR | 17 |
| 1164.742 | 1164.654 | 0.088 | 1 | 9 | 1 | MKLRPLHDR | 18 |
| 1180.792 | 1180.649 | 0.143 | 1 | 9 | 1 | MKLRPLHDR | 19 |
| 1337.882 | 1337.697 | 0.185 | 65 | 77 | 0 | VVFGPYSGSNAIK | 20 |
| 1373.012 | 1372.641 | 0.171 | 3 | 13 | 1 | LRPLHDRVVIR | 21 |
| 1383.002 | 1382.788 | 0.215 | 48 | 60 | 1 | VLDNGEVRALAVK | 22 |
| 1637.202 | 1636.889 | 0.313 | 21 | 37 | 0 | TAGGIVLPGSAAEKPNR | 23 |
| 1737.312 | 1736.909 | 0.403 | 61 | 77 | 1 | VGDKVVFGPYSGSNAIK | 24 |

The protein was then confirmed as being P. aeruginosa derived proteins. Protein spot number 6 was identified as P. aeruginosa GroES (SEQ ID NO: 25).

### EXAMPLE 5

### Use of an Identified P. aeruginosa Protein to Determine a Subject Suffering from a P. aeruginosa Infection

An aliquot of P. aeruginosa GroES was excised from the 2-DE array described in Example 4, washed in H₂O and 1 mM DTT. Proteins were extracted by two successive overnight incubations in 0.1% SDS, 50 mM Tris-HCl pH 7.9, 0.1 mM EDTA, 150 mM NaCl and 5 mM DTT at 4° C. by vigorous shaking, precipitated and resolubilised in 50 μl PBS. Only 6 μl of the extracted proteins were applied to nitrocellulous membrane strips (Biorad, Hercules, Calif., US). Membranes were blocked with 5% (w/v) skim milk in 10 mM Tris-HCl, 100 mM NaCl and 0.2% Tween-20 pH 9.0 prior to use. Anchors were applied to membranes for subsequent localisation of antigenic targets. Crude plasma from healthy controls and CF subjects were diluted 1:3 in PBST buffer (10 mM PBS, 0.05% (v/v) Tween-20) containing 0.5% (w/v) skim milk, and filtered through a 0.22-μm PVDF membrane Millipore). A chemical printer, ChIP™, (Proteome Systems Ltd., Sydney, Australia and Shimadzu, Biotech, Kyoto, Japan) was used to dispense five applications of 0.15 μL 1:3 plasma aliquots onto the immobilised pathogenic protein, PBS and 100 ng BSA. Grid arrays containing 4- or 5-spot positions, where each spot position represented one patient sample, were printed onto targets of membranes. X- and Y-coordinates were established using the software ImagepIQ™ version 1.0 (Proteome Systems Ltd., Sydney, Australia). Approximately 100 μL PBST was used to wash away excess plasma proteins. Bound antibody was detected by printing 0.1 μl HRP-conjugated rabbit anti-human IgG, 1:50000 in PSBT-M buffer (Chemicon Australia Pty., Victoria, Australia). Chemiluminescence was then detected. The size of the printed grid array depended on the area of the immobilised antigenic target, which in current study had a diameter of ˜5 mm.

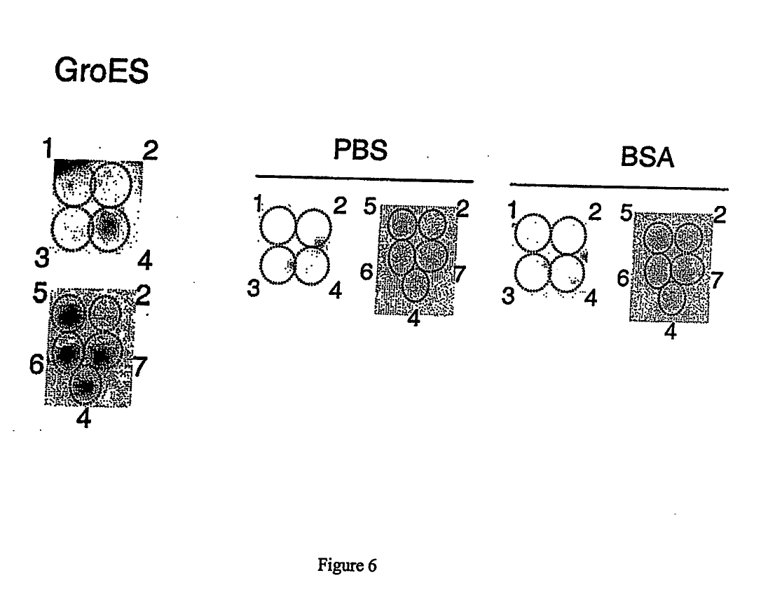

Serological immunoreactivities of five patients were determined towards P. aeruginosa GroES using a chemical printer, ChIP™. As shown in FIG. 6 all screened CF subjects were immunoreactive towards the pathogenic protein, in contrast to the serological non-reactive healthy controls, hence supporting clinically relevant expression of these pathogen-encoded proteins in CF subjects.

### EXAMPLE 6

### Identification of Proteins Recognised in an Autoimmune Response in CF

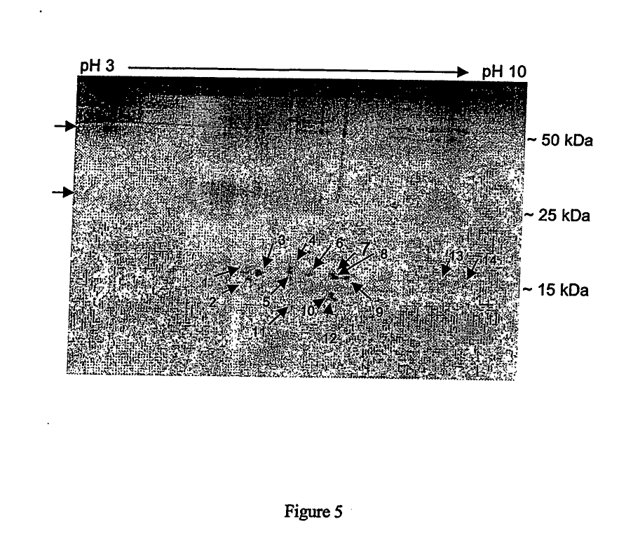

Sputum isolated from CF subjects (described in Example 4) was used in an immunocapture experiment essentially as described in Example 4. As shown in FIG. 5, 14 proteins were isolated from the sputum of CF subjects. Of these 9 have been identified using MS and sequence analysis and shown to be of human origin. MS analysis for spot no. 3, resulted in identification of 6 matching peptides, covering 39% protein. The protein was then identified as human calgranulin B (SEQ ID NO: 26). Spots 1 and 2 were also identified as human calgranulin B. These results suggest that the method of the present invention is useful for the identification of proteins recognised by a subject's own immune system.

### EXAMPLE 7

### Immunization of Chickens to Identify Immunogenic M. tuberculosis Proteins

7.1 Antibody Production

Mycobacterium tuberculosis strain CDC1551 was grown to late-log phase (day 14) in glycerol-alanine-salts (GAS) medium, washed with PBS pH 7.4 and inactivated by gamma-irradiation. Cells are then resuspended (2 g/ml) in PBS buffer containing 8 mM EDTA, protease inhibitors (pepstatin, leupeptin, and PMSF), DNase and RNase afterwhich ˜90% of cells are disrupted by French Press or probe sonication (monitored by acid fast staining). The cell lysate was centrifuged at 3,000 g to pellet unbroken cells and isolate the supernatant. The protein content of each fraction is quantified using the BCA protein assay, and aliquots are stored at −80° C. The culture supernatant was passed through a 0.2 micron filter and the protein content concentrated by Amicon ultrafiltration using a membrane with a molecular weight cutoff of 10,000 Da. The concentrated material is dialyzed against 0.01 M ammonium bicarbonate, quantified with the BCA protein assay, aliquoted, lyophilized, and stored at −80° C. Equal amounts of culture filtrate and whole cell extract was combined and injected into chickens at a concentration of 1 mg/chicken. After four inoculations (one/month) the eggs were collected, broken open and the IgY antibodies harvested by ammonium sulphate precipitation.

7.2 Preparation of Antigen Capture Column

5×10 mg of ammonium sulphate purified IgY antibody was used to generate 5 IgY immobilised CarboLink™ Gels using the Pierce CarboLink™ Kit. Subsequently, the agarose within each gel was pooled to generate a 10 ml bed volume. This pooled column is now referred to as the IgY capture column.

7.3 Affinity Purification of Sputum or Plasma Proteins

7.3.1 Sputum Proteins

A 30 mM final concentration of Iodoacetamide was added to 10.8 ml of sputum from a Chinese TB subject for 2 hours. 10.6 ml of the Iodoacetamide treated sputum was applied to the pooled IgY capture column and incubated overnight at 4 degrees with rotation. The remaining 0.2 ml of sputum was stored at −80° C. and used later in a Bradford protein determination assay. The unbound Chinese sputum antigens (flow through) were removed by centrifugation of column at 4000 g for 10 minutes and saved for 2 subsequent overnight incubations on the same IgY capture column. The IgY capture column was washed with four bed volumes of PBS. Captured proteins were eluted with 50 mM Glycine pH 2.7. The column was regenerated with 3 bed volumes of PBS before addition of flow through for the second out of three overnight incubations. Protein concentrations for flow through, PBS and Glycine washes were determined using a Bradford assay. Eluted proteins were alcohol precipitated and resuspended in 7M Urea, 2M Thiourea, 2% CHAPS and 40 mM Tris. The solubilized proteins were reduced and alkylated with a final concentration of 5 mM tri-n-butylphophine and 10 mM acrylamide respectively. The reduced and alkylated protein sample was washed through a 100 kDa Millipore ultracentrifugation column with 6 volumes of 7M Urea, 2M Thiourea, 2% CHAPS and 40 mM Tris. The 100 kDa flow through was then applied to a 5 kDa Millipore ultracentrifugation column and concentrated to 0.3 ml and used in two-dimensional gel electrophoresis.

7.3.2 Plasma Proteins

10.6 ml of Chinese plasma was applied to the pooled IgY capture column and incubated overnight at 4 degrees with rotation. The unbound Chinese plasma antigens (flow through) were removed by centrifugation of column at 4000 g for 10 minutes and saved for 2 subsequent overnight incubations on the same IgY capture column. The IgY capture column was washed with four bed volumes of PBS. Captured proteins were eluted with 50 mM Glycine pH 2.7. The column was regenerated with 3 bed volumes of PBS before addition of flow through for the second out of three overnight incubations. Protein concentrations for flow through, PBS and Glycine washes were determined using a Bradford assay. Eluted proteins were alcohol precipitated and resuspended in 7M Urea, 2M Thiourea, 2% CHAPS and 40 mM Tris. The solubilized proteins were reduced and alkylated with a final concentration of 5 mM tri-n-butylphophine and 10 mM acrylamide respectively.

7.4 Two-Dimensional Gel Electrophoresis

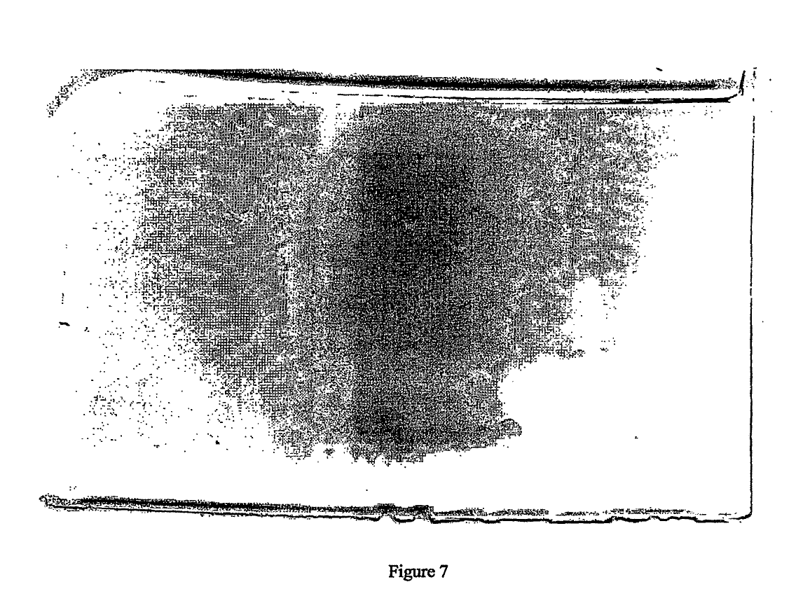

Eleven centimetre pH 3-10 or pH 4-7 IPGs were purchased from Amersham (Uppsala, Sweden). Isoelectric focusing was conducted as per manufacturer's instructions using an IsoElectrIQ² unit from Proteome Systems (Woburn, Mass.). Second dimension 6-15% or 14% homogenous Tris-Acetate Gelchip gels were run as recommended by manufacturer (Proteome Systems, Woburn, Mass.). Arrayed proteins were visualised by silver-staining (Shevchenko et al., Mass spectrometric sequencing of proteins silver-stained polyacrylamide gels. 68, 850-858. 1996) or transferred to PVDF-P membranes (Millipore, Billerica, Mass.) by using semi-wet membrane-blotting cassettes accompanying the IsoElectrIQ² unit from Proteome Systems (Woburn, US). An example gel showing protein isolated from sputum of a subject suffering from tuberculosis using immunoglobulin from an immunized chicken is shown in FIG. 7.

### EXAMPLE 8

### Identification of an Immunogenic Ovarian Cancer Protein

Both tumor tissue and serum are collected from a number of subjects suffering from ovarian cancer. All tumor samples are confirmed to be of epithelial origin.

8.1 Preparation of Protein from Tumor Cells

Cells are dissociated in cold PBS, pelleted, and washed twice in cold PBS. For the preparation of total cell protein extracts, cells are resuspended in extraction buffer (125 mM Tris-HCl, pH 6.8, 3% SDS, 5% 2-mercaptoethanol, 1 mM PMSF, and 2.5 g/ml of leupeptin, pepstain, aprotinin, antipain, and chymostatin), and sonicated. For the preparation of soluble and insoluble cell extracts, cells are resuspended in cold buffer (10 mM sodium phosphate buffer, pH 8.0, 140 mM NaCl, 3 mM MgCl₂, 1 mM DTT, 0.5% Nonidet P40, and protease inhibitors as above), incubated on ice for 15 min, and centrifuged at 10,000 rpm for 5 min. Supernatants are removed as soluble extracts, and pellets are resuspended in cold buffer (10 mM sodium phosphate buffer, pH 7.5, 150 mM NaCl, 1 mM DIT, 1.0% Nonidet P40, 0.1% SDS, and protease inhibitors as above), vortexed, and then centrifuged at 10,000 rpm for 5 min. Resulting supernatants are removed as insoluble extracts.

8.2 Preparation of an Immuno-Capture Column.

An immuno-capture column is produced by pooling approximately 5 mL serum from five subjects suffering from epithelial ovarian cancer (total protein concentration of ˜40 mg/mL). Immunoglobulin is bound to Protein A Sepharose by incubating the pooled plasma with 10 mL 50% slurry of Protein A Sepharose. The matrix is washed in 10 mM PBS pH 7.4 and bound immunoglobulin irreversibly immobilised utilizing DSS essentially as described by the manufacturer (Pierce). The generated column is referred to as the capture column.

8.3 Isolation of an Immunogenic Protein

The capture column is incubated overnight with the isolated breast tumor protein (described in Example 7.1) at 4° C. at constant rotation. Beads are subsequently collected by centrifugation. The flow-through is collected and saved for subsequent incubation steps (the protein extract is passed over the capture column a number of times in each capture). The harvested beads are washed 3 times in 10 mM PBS pH 7.4 and captured proteins eluted with 50 mM glycine pH 2.7. The column is extensively washed with first 50 mM glycine pH 2.7 then 10 mM PBS pH 7.2 prior subsequent incubation steps.

Eluted proteins are alcohol precipitated and subsequently resolubilised using the ProteoPrep Universal Extraction kit (Sigma, St. Louis, Mo.) essentially as described by the manufacturer. Proteins are then reduced and alkylated with a final concentration of 5 mM tri-n-butylphophine and 10 mM acrylamide, respectively.

8.4 Two-Dimensional Gel Electrophoresis

Eleven centimetre pH 3-10 or pH 4-7 IPGs (Amersham, Uppsala, Sweden) are used. Isoelectric focusing is conducted as per manufacturer's instructions using an IsoElectrIQ² unit from Proteome Systems (Woburn, Mass.). Second dimension 6-15% or 14% homogenous Tris-Acetate Gelchip gels are run as recommended by manufacturer (Proteome Systems, Woburn, Mass.). Arrayed proteins are visualised by silver-staining (Shevchenko et al., Mass spectrometric sequencing of proteins silver-stained polyacrylamide gels. 68, 850-858. 1996).

8.5 MS Analysis

Proteins spots of interest are excised and washed twice in 100 mM NH₄HCO₃: 50% acetonitrile (ACN) pH 8.2 and dehydrated at 50° C. for 30 minutes. Proteins are digested as described by Katayama et al (Improvement of in-gel digestion protocol for peptide mass fingerprinting by matrix-assisted laser desorption/ionization time-of-flight mass spectrometry) and digested for 3 hours at 37° C. Tryptic peptides are extracted by sonication and purified as described by Kussmann et al. Peptides are eluted with ˜1.5 μl MALDI matrix solution (70% ACN, 0.1% TFA, 1.5 mg/ml alpha-cyano-4-hydroxycinnamic acid (Sigma, St. Louis, Mo.). Peptide mass fingerprints (PMF) are generated by matrix-assisted laser desorption/ionisation-time-of-flight-mass spectrometry (MALDI-TOF-MS) using an Axima CFR (Kratos, Manchester, UK) or an ABI MALDI MS/MS (AME Bioscience, London, UK).

Using this method a fragment of an immunogenic protein is identified and compared to databases to identify the immunogenic protein.

### EXAMPLE 9

### Immunization of Mice with Breast Cancer Cells to Determine an Immunogenic Protein

9.1 Immunization of Mice

A number of mice are immunized with MCF-7 breast cancer cell lines. The MCF-7 cells are mixed with Freund's complete adjuvant (manufactured by DIFCO) in a 1:1 ratio and the mixture emulsified. The emulsion is then injected into female Balb/C mice subcutaneously.

Booster immunizations of an emulsified mixture of the cell solution and Freund's incomplete adjuvant (manufactured by DIFCO) (in a 1:1 ratio) are administered by injection at about 2 week intervals by subcutaneous injection. Three days after the third booster, a blood sample is collected from the tail vein and antibody production in the serum is measured by a direct solid phase ELISA.

MCF-7 cells are diluted with PBS and the resulting solution adsorbed to an ELISA plate for approximately 2 hours. The plate is then blocked by a 4-fold dilution of Blockace (manufactured by Snow Brand Milk Products) in PBS. After washing the plate, various dilutions of the serum obtained from the immunized mice in a serum diluting buffer (PBS containing 5% FBS) are added to each well of the plate and incubated at room temperature for 2 hours.

Following washing the plate alkaline phosphatase labelled mouse IgG antibody (manufactured by ICN/Cappel) is added to each well of the plate and incubated at room temperature for approximately 1 hour.

Disodium p-nitrophenyl phosphate (SIGMA) is then dissolved in a substrate reaction mixture (9.6% diethanolamine buffer containing 0.5 mM magnesium chloride, pH 9.7) at concentration of approximately 2 mg/ml to prepare a substrate solution. The plate is washed 7 times with purified water and the substrate solution added thereto. After reaction with the substrate solution, 3N NaOH is added to stop the reaction and the absorbance at 405 nm is measured.

Serum is extracted from those mice that have developed an immune response to the MCF-7 cells and the serum pooled.

9.2 Preparation of an Immunocapture Column

The recombinant immunoglobulin binding protein PAM protein A mimetic) is used to isolate immunoglobulin from serum samples.

PAM (Arg-Thr-Tyr)₄-Lys₂-Lys-Gly, (SEQ ID NO: 5) are produced by solid phase peptide synthesis following the Fmoc methodology on a fully automated peptide synthesizer 431A (Perkin-Elmer) as described previously (Fassina et al J. Mol. Recognit. 9: 564, 1996). After resin cleavage, peptides are purified by reversed-phase high performance liquid chromatography (RP-HPLC) and their identity confirmed by amino acid analysis and time of flight matrix assisted laser desorption ionization (TOF-MALDI) mass spectrometry, which provided a molecular weight identical to the expected value (2141 amu).

Peptides are coupled to Emphaze matrix (polyacrylamide/azlactone-activated gel) (TECNOGEN, Piana di MonteVema, CE, Italy), as recommended by the manufacturer's protocols. Ten milligrams of peptide is dissolved in 6 ml of 0.2 M NaHCO₃, 0.6 M Na citrate, pH 8.0, and incubated with 130 mg (corresponding to 1 ml) of pre-activated matrix. The suspension is incubated for several hours at room temperature under gentle agitation, by monitoring the extent of peptide incorporation by RP-HPLC analysis at different times. After washing with 0.1 M Tris, pH 8.5, to deactivate residual active groups, the resin is finally packed into a 100×6.6 mm I.D. glass column.

Samples, corresponding to 0.5-1 ml of serum (Example 9.1) are diluted and loaded onto the column equilibrated at a flow rate of 60 cm/h with buffer. Bound fraction is immediately neutralized with a few drops of 1 M Tris, pH 9.5 and characterized by SDS-PAGE and gel-permeation analysis to determine IgG purity, and by enzyme-linked immunosorbent assay (ELISA), to evaluate the immunoreactivity and the amount of recovered immunoglobulins.

The immunoglobulin bound to the column is then crosslinked using DPS (Pierce) essentially following manufacturer's instructions

9.3 Preparation of Protein from Tumor Cells

MCF-7 cells are collected in cold PBS, pelleted, and washed twice in cold PBS. For the preparation of total cell protein extracts, cells are resuspended in extraction buffer (125 mM Tris-HCl, pH 6.8, 3% SDS, 5% 2-mercaptoethanol, 1 mM PMSF, and 2.5 g/ml of leupeptin, pepstain, aprotinin, antipain, and chymostatin), and sonicated. For the preparation of soluble and insoluble cell extracts, cells are resuspended in cold buffer (10 mM sodium phosphate buffer, pH 8.0, 140 mM NaCl, 3 mM MgCl₂, 1 mM DTT, 0.5% Nonidet P-40, and protease inhibitors as above), incubated on ice for 15 min, and centrifuged at 10,000 rpm for 5 min. Supernatants are removed as soluble extracts, and pellets are resuspended in cold buffer (10 mM sodium phosphate buffer, pH 7.5, 150 mM NaCl, 1 mM DTT, 1.0% Nonidet P-40, 0.1% SDS, and protease inhibitors as above), vortexed, and then centrifuged at 10,000 rpm for 5 min. Resulting supernatants are removed as insoluble extracts.

9.4 Isolation of an Immunogenic Protein

The capture column is incubated overnight with the isolated MCF-7 protein (described in Example 9.3) at 4° C. at constant rotation. Beads are subsequently collected by centrifugation. The flow-through is collected and saved for subsequent incubation steps (the protein extract is passed over the capture column a number of times in each capture). The harvested beads are washed 3 times in 10 mM PBS pH 7.4 and captured proteins eluted with 50 mM glycine pH 2.7. The column is extensively washed with first 50 mM glycine pH 2.7 then 10 mM PBS pH 7.2 prior subsequent incubation steps.

Eluted proteins are alcohol precipitated and subsequently resolubilised using the ProteoPrep Universal Extraction kit (Sigma, St. Louis, Mo.) essentially as described by the manufacturer. Proteins are then reduced and alkylated with a final concentration of 5 mM tri-n-butylphophine and 10 mM acrylamide, respectively.

Collected proteins are isolated and analysed using MS essentially as described in Example 8.

### EXAMPLE 10

### Identification of an Immunogenic Protein in Diabetes

10.1 Preparation of an Immuno-Capture Column.

An immuno-capture column is produced by pooling approximately 5 mL serum from a number of subjects suffering from type-I diabetes (total protein concentration of ˜40 mg/mL). Immunoglobulin is bound to Protein G Sepharose by incubating the pooled plasma with 10 mL 50% slurry of Protein G Sepharose. The matrix is washed in 10 mM PBS pH 7.4 and bound immunoglobulin irreversibly immobilised utilizing DSS essentially as described by the manufacturer (Pierce). The generated column is referred to as the capture column.

10.2 Identification of an Immunogenic Protein from Diabetes

Pancreatic islet like cells are produced essentially as described in Lumelsky et al Science, 292:1389, 2001. These cells are then lysed and protein extracted. Differentiated cells are lysed and protein extracted using the ProteoPrep Universal Extraction kit (Sigma, St. Louis, Mo.) essentially as described by the manufacturer.

The capture column is incubated overnight with the isolated differentiated cell protein or with serum isolated from a diabetic subject or a non-diabetic subject at 4° C. at constant rotation. Beads are subsequently collected by centrifugation. The flow-through is collected and saved for subsequent incubation steps (the protein extract is passed over the capture column a number of times in each capture). The harvested beads are washed 3 times in 10 mM PBS pH 7.4 and captured proteins eluted with 50 mM glycine pH 2.7. The column is extensively washed with first 50 mM glycine pH 2.7 then 10 mM PBS pH 7.2 prior subsequent incubation steps.

Eluted proteins are alcohol precipitated and subsequently resolubilised using the ProteoPrep Universal Extraction kit (Sigma, St. Louis, Mo.) essentially as described by the manufacturer. Proteins are then reduced and alkylated with a final concentration of 5 mM tri-n-butylphophine and 10 mM acrylamide, respectively.

Collected proteins are isolated and analysed using MS essentially as described in Example 8.

### EXAMPLE 11

### Identification of an Immunogenic Protein from Hepatitis

11.1 Production of Hepatitis C Virus Like Particles.

Construction of recombinant baculovirus bvHCV.Sp7 containing the complementary DNA for the HCV structural proteins (genotype Ib J strain) is performed essentially as described in Triyatni et al J Virol; 76:9335-9344, 2002. HCV-LPs are partially purified by sucrose gradient centrifugation as described previously Tryatni supra.

11.2 Immunization of Mice.

Mice (6-8 weeks old) are immunized 4 times at 3-week intervals with 20 μg of HCV-LPs into each quadriceps muscle in a total volume of 100 μL with either the AS01B adjuvant (GalaxoSmithKline) or CpG 10105 (Coley Pharmaceutical Group).

Mice that produce antibodies to the particles are determined by an enzyme-linked immunosorbent assay. Blood samples before immunization and 2 weeks after each immunization are collected from the tail vein and analyzed for HCV E1/E2 antibodies by enzyme-linked immunosorbent assay essentially as described in Lechmann et al., Hepatology; 34:417-423, 2001.

11.3 Production of an Immunocapture Column

An immuno-capture column is produced by pooling approximately serum from a number of mice producing anti-HCV E1/E2 antibodies. Immunoglobulin is bound to PAM-EMPHAZE (produced as described in Example 9.2) Bound immunoglobulin irreversibly immobilised utilizing DSS essentially as described by the manufacturer (Pierce). The generated column is referred to as the capture column.

11.4 Identification of an Immunogenic Protein from Hepatatis

The capture column is incubated overnight with the partially purified HCV-LPs at 4° C. at constant rotation. The flow-through is collected and saved for subsequent incubation steps (the protein extract is passed over the capture column a number of times in each capture). The harvested beads are washed 3 times in 10 mM PBS pH 7.4 and captured proteins eluted with 50 mM glycine pH 2.7. The column is extensively washed with first 50 mM glycine pH 2.7 then 10 mM PBS pH 7.2 prior subsequent incubation steps.

Eluted proteins are alcohol precipitated and subsequently resolubilised using the ProteoPrep Universal Extraction kit (Sigma, St. Louis, Mo.) essentially as described by the manufacturer. Proteins are then reduced and alkylated with a final concentration of mM tri-n-butylphophine and 10 mM acrylamide, respectively.

Collected proteins are isolated and analysed using MS essentially as described in Example 8.

1. A method for identifying an immunogenic protein or fragment thereof capable of eliciting an immune response, said method comprising obtaining a protein complex comprising an immunoglobulin or mixtures thereof or an immunoglobulin-containing fraction from a subject or a cell, tissue or organ thereof and identifying a protein or fragment thereof bound to the immunoglobulin by virtue of an antigen antibody interaction, thereby identifying an immunogenic protein or fragment thereof capable of eliciting an immune response.

2. The method according to claim 1 further comprising obtaining a sample from the subject that comprises the protein complex or mixture thereof or immunoglobulin-containing fraction.

3. The method according to claim 2 further comprising obtaining one or more immunoglobulin-containing fractions from the sample.

4-10. (canceled)

11. The method according to claim 1 wherein the protein complex or immunoglobulin-containing fraction is obtained by a process comprising contacting the sample with one or more compounds capable of binding an immunoglobulin for a time and under conditions sufficient for binding to occur and isolating the compound, wherein the one or more compounds is/are previously immobilized on a solid support, matrix or resin.

12-13. (canceled)

14. The method according to claim 11 further comprising washing the one or more immobilized compounds to thereby remove non-specifically bound or unbound protein.

15-16. (canceled)

17. The method according to claim 1 wherein the subject suffers from a chronic infection or has suffered previously from a chronic infection.

18-19. (canceled)

20. The method according to claim 17 wherein the infection is selected from the group consisting of a viral infection, a bacterial infection, a yeast infection, a fungal infection and a parasitic infection.

21. The method according to claim 20 wherein the infection is a bacterial infection.

22. The method according to claim 21 wherein the bacterial infection is a Pseudomonas infection.

23. The method according to claim 22 wherein the bacterial infection is a Mycobacterium infection.

24. The method according to claim 17 wherein the infection is a pulmonary infection.

25. The method according to claim 24 wherein the pulmonary infection is caused by or associated with the presence of Pseudomonas aeruginosa or Mycobacterium tuberculosis.

26. The method according to claim 1 wherein the subject suffers from an autoimmune condition associated with an inflammatory condition.

27-36. (canceled)

37. The method according to claim 11 further comprising linking immunoglobulin to the one or more compounds.

38. The method according to claim 37 wherein linking comprises performing a process that comprises contacting a cross-linking agent with the one or more compounds having immunoglobulin bound thereto for a time and under conditions sufficient for covalent linkage to occur between a compound and immunoglobulin.

39-42. (canceled)

43. The method according to claim 1 wherein the A method for identifying an immunogenic protein or immunogenic protein fragment of an agent that causes a disease or disorder in a subject comprising:
(i) obtaining a protein complex comprising an immunoglobulin or mixtures thereof or an immunoglobulin-containing fraction from a subject suffers suffering from a the disease or disorder or has having suffered previously from the disease or disorder said method further comprising or a cell, tissue or organ thereof;
(ii) contacting immunoglobulin in the protein complex or immunoglobulin-containing fraction with a sample comprising an the agent that causes the disease or disorder or a derivative thereof; and
(ii) identifying a protein or fragment thereof bound to said immunoglobulin by virtue of an antigen-antibody interaction,
wherein the identified protein is an immunogenic protein or immunogenic protein fragment of an agent that causes a disease or disorder in a subject.

44-64. (canceled)

65. The method according to claim 43 wherein the sample comprises a protein or cellular extract of the agent that causes the disease or disorder.

66. The method according to claim 43 wherein the agent that causes the disease or disorder is an infectious agent selected from the group consisting of a virus, a bacterium, a yeast, a fungus and a parasite.

67. (canceled)

68. The method according to claim 67 wherein the infectious agent is Pseudomonas aeruginosa or Mycobacterium tuberculosis.

69-94. (canceled)

95. The method according to claim 1 wherein the subject suffers from an autoimmune condition associated with an inflammatory condition said method additionally comprising contacting immunoglobulin in the protein complex or immunoglobulin-containing fraction with a sample comprising protein from a subject suffering from an autoimmune condition.

96-119. (canceled)

120. The method according to claim 95 wherein the subject suffers from cystic fibrosis.

121. The method according to claim 120 wherein the subject is suffering from or has previously suffered from an acute pulmonary exacerbation.

122. (canceled)

123. The method according to claim 120 wherein the subject additionally suffers from an infection.

124-125. (canceled)

126. The method according to claim 125 wherein the subject suffers from an infection caused by Pseudomonas aeruginosa.

127-166. (canceled)

167. The method according to claim 1 additionally comprising separating an immunogenic protein or fragment thereof bound to the immunoglobulin by virtue of an antigen antibody interaction from the immunoglobulin.

168. The method according to claim 167 wherein the immunogenic protein or fragment thereof is separated from the immunoglobulin by a method that comprises contacting the protein complex or immunoglobulin-containing fraction with a compound that disrupts the antigen-antibody interaction for a time an under conditions sufficient to disrupt the antigen-antibody interaction.

169. The method according to claim 168 wherein the compound that disrupts the antigen-antibody interaction is selected from the group consisting a compound that modulates the pH of the immunoglobulin fraction, a salt, an ionic detergent, a dissociating agent and a chaotropic agent.

170. The method according to claim 1 additionally comprising isolating a protein that is or was bound to the immunoglobulin-containing fraction by virtue of an antigen-antibody interaction.

171. The method according to claim 170 wherein the protein is isolated by performing two-dimensional gel electrophoresis.

172. (canceled)

173. The method according to claim 1 wherein a protein that is or was bound to the immunoglobulin-containing fraction by virtue of an antigen-antibody interaction is identified using mass spectrometry.

174. (canceled)

175. A method comprising:
(a) obtaining a protein complex comprising an immunoglobulin or mixtures thereof or an immunoglobulin-containing fraction from a subject that has raised an immune response against an immunogenic protein or fragment thereof or a cell, tissue or organ thereof by a method comprising contacting a sample from the subject with one or more compounds capable of binding an immunoglobulin for a time and under conditions sufficient for binding to occur and isolating the one or more compounds;
(b) linking immunoglobulin in the protein complex or immunoglobulin-containing fraction to the one or more compounds;
(c) separating an immunogenic protein or fragment thereof from the linked immunoglobulin;
(d) contacting a sample comprising the immunogenic protein or fragment thereof with the linked immunoglobulin;
(e) optionally, separating the immunogenic protein or fragment thereof from the linked immunoglobulin;
(f) repeating (d) and (e) one or more times; and
(g) identifying a protein or fragment thereof separated from the immunoglobulin, thereby identifying an immunogenic protein or fragment thereof.

176-177. (canceled)

178. The method according to claim 175 wherein (d) contacting a sample comprising the immunogenic protein or fragment thereof with the linked immunoglobulin and (e) separating the immunogenic protein or fragment thereof from the linked immunoglobulin are repeated a sufficient number of times to identify one or more immunogenic proteins.

179-198. (canceled)

199. The method according to claim 37 additionally comprising:
(a) separating an immunogenic protein or fragment thereof from the linked immunoglobulin;
(b) contacting a sample comprising the immunogenic protein or fragment thereof with the linked immunoglobulin;
(c) separating the immunogenic protein or fragment thereof from the linked immunoglobulin;
(d) optionally, repeating (b) and (c) one or more times; and
(e) identifying a protein or fragment thereof separated from the immunoglobulin, thereby identifying an immunogenic protein or fragment thereof bound to an immunoglobulin by an antigen antibody interaction.

200. The method according to claim 199 wherein (b) contacting a sample comprising the immunogenic protein or fragment thereof with the linked immunoglobulin and (c) separating the immunogenic protein or fragment thereof from the linked immunoglobulin are repeated a sufficient number of times to identify one or more immunogenic proteins.

201. The method according to claim 1 wherein the immunogenic protein or fragment thereof is identified by determining the amino acid sequence of the protein or fragment thereof.

202. The method according to claim 43 additionally comprising immobilizing the protein complex or immunoglobulin-containing fraction prior to contacting the protein complex or immunoglobulin-containing fraction with the sample.

203. The method according to claim 95 additionally comprising immobilizing the protein complex or immunoglobulin-containing fraction prior to contacting the protein complex or immunoglobulin-containing fraction with the sample.
